SEQUENCE LISTING (19 sequences)

SEQ ID NO: 1 - protein, 595 aa, Populus alba
MATELLCLHRPISLTHKLFRNPLPKVIQATPLTLKLRCSVSTENVSFTETETEARRSANYEPNSWDYDYLLSSDTDESIEVYKDKAKKLEAEVRREINNEKAEFLTLLELIDNVQRLGLGYRFESDIRGALDRFVSSGGFDAVTKTSLHGTALSFRLLRQHGFEVSQEAFSGFKDQNGNFLENLKEDIKAILSLYEASFLALEGENILDEAKVFAISHLKELSEEKIGKELAEQVNHALELPLHRRTQRLEAVWSIEAYRKKEDANQVLLELAILDYNMIQSVYQRDLRETSRWWRRVGLATKLHFARDRLIESFYWAVGVAFEPQYSDCRNSVAKMFSFVTIIDDIYDVYGTLDELELFTDAVERWDVNAINDLPDYMKLCFLALYNTINEIAYDNLKDKGENILPYLTKAWADLCNAFLQEAKWLYNKSTPTFDDYFGNAWKSSSGPLQLVFAYFAVVQNIKKEEIENLQKYHDTISRPSHIFRLCNDLASASAEIARGETANSVSCYMRTKGISEELATESVMNLIDETWKKMNKEKLGGSLFAKPFVETAINLARQSHCTYHNGDAHTSPDELTRKRVLSVITEPILPFER

SEQ ID NO: 2 - protein, 559 aa, Populus alba
MCSVSTENVSFTETETEARRSANYEPNSWDYDYLLSSDTDESIEVYKDKAKKLEAEVRREINNEKAEFLTLLELIDNVQRLGLGYRFESDIRGALDRFVSSGGFDAVTKTSLHGTALSFRLLRQHGFEVSQEAFSGFKDQNGNFLENLKEDIKAILSLYEASFLALEGENILDEAKVFAISHLKELSEEKIGKELAEQVNHALELPLHRRTQRLEAVWSIEAYRKKEDANQVLLELAILDYNMIQSVYQRDLRETSRWWRRVGLATKLHFARDRLIESFYWAVGVAFEPQYSDCRNSVAKMFSFVTIIDDIYDVYGTLDELELFTDAVERWDVNAINDLPDYMKLCFLALYNTINEIAYDNLKDKGENILPYLTKAWADLCNAFLQEAKWLYNKSTPTFDDYFGNAWKSSSGPLQLVFAYFAVVQNIKKEEIENLQKYHDTISRPSHIFRLCNDLASASAEIARGETANSVSCYMRTKGISEELATESVMNLIDETWKKMNKEKLGGSLFAKPFVETAINLARQSHCTYHNGDAHTSPDELTRKRVLSVITEPILPFER

SEQ ID NO: 3 - protein, 608 aa, Pueraria montana var. lobata
MATNLLCLSNKLSSPTPTPSTRFPQSKNFITQKTSLANPKPWRVICATSSQFTQITEHNSRRSANYQPNLWNFEFLQSLENDLKVEKLEEKATKLEEEVRCMINRVDTQPLSLLELIDDVQRLGLTYKFEKDIIKALENIVLLDENKKNKSDLHATALSFRLLRQHGFEVSQDVFERFKDKEGGFSGELKGDVQGLLSLYEASYLGFEGENLLEEARTFSITHLKNNLKEGINTKVAEQVSHALELPYHQRLHRLEARWFLDKYEPKEPHHQLLLELAKLDFNMVQTLHQKELQDLSRWWTEMGLASKLDFVRDRLMEVYFWALGMAPDPQFGECRKAVTKMFGLVTIIDDVYDVYGTLDELQLFTDAVERWDVNAINTLPDYMKLCFLALYNTVNDTSYSILKEKGHNNLSYLTKSWRELCKAFLQEAKWSNNKIIPAFSKYLENASVSSSGVALLAPSYFSVCQQQEDISDHALRSLTDFHGLVRSSCVIFRLCNDLATSAAELERGETTNSIISYMHENDGTSEEQAREELRKLIDAEWKKMNRERVSDSTLLPKAFMEIAVNMARVSHCTYQYGDGLGRPDYATENRIKLLLIDPFPINQLMYV

SEQ ID NO: 4 - protein, 564 aa, Pueraria montana var. lobata
MCATSSQFTQITEHNSRRSANYQPNLWNFEFLQSLENDLKVEKLEEKATKLEEEVRCMINRVDTQPLSLLELIDDVQRLGLTYKFEKDIIKALENIVLLDENKKNKSDLHATALSFRLLRQHGFEVSQDVFERFKDKEGGFSGELKGDVQGLLSLYEASYLGFEGENLLEEARTFSITHLKNNLKEGINTKVAEQVSHALELPYHQRLHRLEARWFLDKYEPKEPHHQLLLELAKLDFNMVQTLHQKELQDLSRWWTEMGLASKLDFVRDRLMEVYFWALGMAPDPQFGECRKAVTKMFGLVTIIDDVYDVYGTLDELQLFTDAVERWDVNAINTLPDYMKLCFLALYNTVNDTSYSILKEKGHNNLSYLTKSWRELCKAFLQEAKWSNNKIIPAFSKYLENASVSSSGVALLAPSYFSVCQQQEDISDHALRSLTDFHGLVRSSCVIFRLCNDLATSAAELERGETTNSIISYMHENDGTSEEQAREELRKLIDAEWKKMNRERVSDSTLLPKAFMEIAVNMARVSHCTYQYGDGLGRPDYATENRIKLLLIDPFPINQLMYV

SEQ ID NO: 5 - protein, 595 aa, Salix sp. DG-2011
MATELLCLHRPISLTPKLFRNPLPKVILATPLTLKLRCSVSTENVSFTETETETRRSANYEPNSWDYDYLLSSDTDESIEVYKDKAKKLEAEVRREINNEKAEFLTLLELIDNVQRLGLGYRFESDIRRALDRFVSSGGFDAVTKTSLHATALSFRFLRQHGFEVSQEAFGGFKDQNGNFLENLKEDIKAILSLYEASFLALEGENILDEAKVFAISHLKELSEEKIGKDLAEQVNHALELPLHRRTQRLEAVWSIEAYRKKEDANQVLLELAILDYNMIQSVYQRDLRETSRWWRRVGLATKLHFARDRLIESFYWAVGVAFEPQYSDCRNSVAKMFSFVTIIDDIYDVYGTLDELELFTDAVERWDVNAINDLPDYMKLCFLALYNTINEIAYDNLKEKGENILPYLTKAWADLCNAFLQEAKWLYNKSTPTFDDYFGNAWKSSSGPLQLVFAYFAVVQNIKKEEIENLQKYHDIISRPSHIFRLCNDLASASAEIARGETANSVSCYMRTKGISEELATESVMNLIDETWKKMNKEKLGGSLFPKPFVETAINLARQSHCTYHNGDAHTSPDELTRKRVLSVITEPILPFER

SEQ ID NO: 6 - protein, 559 aa, Salix sp. DG-2011
MCSVSTENVSFTETETETRRSANYEPNSWDYDYLLSSDTDESIEVYKDKAKKLEAEVRREINNEKAEFLTLLELIDNVQRLGLGYRFESDIRRALDRFVSSGGFDAVTKTSLHATALSFRFLRQHGFEVSQEAFGGFKDQNGNFLENLKEDIKAILSLYEASFLALEGENILDEAKVFAISHLKELSEEKIGKDLAEQVNHALELPLHRRTQRLEAVWSIEAYRKKEDANQVLLELAILDYNMIQSVYQRDLRETSRWWRRVGLATKLHFARDRLIESFYWAVGVAFEPQYSDCRNSVAKMFSFVTIIDDIYDVYGTLDELELFTDAVERWDVNAINDLPDYMKLCFLALYNTINEIAYDNLKEKGENILPYLTKAWADLCNAFLQEAKWLYNKSTPTFDDYFGNAWKSSSGPLQLVFAYFAVVQNIKKEEIENLQKYHDIISRPSHIFRLCNDLASASAEIARGETANSVSCYMRTKGISEELATESVMNLIDETWKKMNKEKLGGSLFPKPFVETAINLARQSHCTYHNGDAHTSPDELTRKRVLSVITEPILPFER

SEQ ID NO: 7 - protein, 639 aa, Methylococcus capsulatus
MTETKRYALLEAADHPAALRNLPEDRLPELAEELRGYLLESVSRSGGHLAAGLGTVELTIALHYVFNTPEDKLVWDVGHQAYPHKILTGRRARLPTIRKKGGLSAFPNRAESPYDCFGVGHSSTSISAALGMAVAAALERRPIHAVAIIGDGGLTGGMAFEALNHAGTLDANLLIILNDNEMSISPNVGALNNYLAKILSGKFYSSVRESGKHLLGRHMPGVWELARRAEEHVKGMVAPGTLFEELGFNYFGPIDGHDLDTLITTLRNLRDQKGPRFLHVVTRKGKGYAPAEKDPVAYHGVGAFDLDADELPKSKPGTPSYTEVFGQWLCDMAARDRRLLGITPAMREGSGLVEFSQRFPDRYFDVGIAEQHAVTFAAGQASEGYKPVVAIYSTFLQRAYDQLIHDVALQNLPVLFAIDRAGLVGPDGPTHAGSFDLSFMRCIPNMLIMAPSDENECRQMLYTGFIHDGPAAVRYPRGRGPGVRPEETMTAFPVGKGEVRLRGKGTAILAFGTPLAAALAVGERIGATVANMRFVKPLDEALILELAATHDRIVTVEENAIAGGAGSAVGEFLAAQHCGIPVCHIGLKDEFLDQGTREELLAIAGLDQAGIARSIDAFIQATAAADKPRRARGQAKDKH

SEQ ID NO: 8 - protein, 394 aa, Methylococcus capsulatus
MKGICILGSTGSIGVSTLDVLARHPDRYRVVALSANGNVDRLFEQCRAHRPRYAAVIRAEAAACLRERLMAAGLGGIEVLAGPEALEQIASLPEVDSVMAAIVGAAGLLPTLAAARAGKDVLLANKEALVMSGPLFMAEVARSGARLLPIDSEHNAVFQCMPAAYRAGSRAVGVRRILLTASGGPFLHTPLAELETVTPEQAVAHPNWVMGRKISVDSATMMNKGLEVIEACLLFNAKPDDVQVVVHRQSVIHSMVDYVDGTVLAQMGTPDMRIPIAHALAWPDRFESGAESLDLFAVRQLNFERPDLARFPCLRLAYEAVGAGGTAPAILNAANETAVAAFLDRRLAFTGIPRVIEHCMARVAPNAADAIESVLQADAETRKVAQKYIDDLRV

SEQ ID NO: 9 - protein, 234 aa, Methylococcus capsulatus
MSTDARFWIVVPAAGVGKRMGADIPKQYLDVAGKPVLQHTLERLLSVRRVTAVMVALGANDEFWPELPCSREPRVLATTGGRERADSVLSALTALAGRAADGDWVLVHDAARLCVTRDDVERLMETLEDDPVGGILALPVTDTLKTVENGTIQGSADRSRVWRALTPQMFRYRALKEALEAAARRGLTVTDEASALELAGLSPRVVEGRPDNIKITRPEDLPLAAFYLERQCFE

SEQ ID NO: 10 - protein, 291 aa, Methylococcus capsulatus
MDRRESSVMKSPSLRLPAPAKLNLTLRITGRRPDGYHDLQTVFQFVDVCDWLEFRADASGEIRLQTSLAGVPAERNLIVRAARLLKEYAGVAAGADIVLEKNLPMGGGLGGGSSNAATTLVALNRLWDLGLDRQTLMNLGLRLGADVPIFVFGEGAWAEGVGERLQVLELPEPWYVIVVPPCHVSTAEIFNAPDLTRDNDPITIADFLAGSHQNHCLDAVVRRYPVVGEAMCVLGRYSRDVRLTGTGACVYSVHGSEEEAKAACDDLSRDWVAIVASGRNLSPLYEALNER

SEQ ID NO: 11 - protein, 158 aa, Methylococcus capsulatus
MFRIGQGYDAHRFKEGDHIVLCGVKIPFGRGFAAHSDGDVALHALCDALLGAAALGDIGRHFPDTDARYKGIDSRVLLREVRQRIASLGYTVGNVDVTVVAQAPRLAAHIQAMRENLAQDLEIPPDCVNVKATTTEGMGFEGRGEGISAHAVALLARR

SEQ ID NO: 12 - protein, 407 aa, Methylococcus capsulatus
MMNRKQTVGVRVGSVRIGGGAPIVVQSMTNTDTADVAGTVRQVIDLARAGSELVRITVNNEEAAEAVPRIREELDRQGCNVPLVGDFHFNGHKLLDKYPACAEALGKFRINPGNVGRGSKRDPQFAQMIEFACRYDKPVRIGVNWGSLDQSVLARLLDENARLAEPRPLPEVMREAVITSALESAEKAQGLGLPKDRIVLSCKMSGVQELISVYEALSSRCDHALHLGLTEAGMGSKGIVASTAALSVLLQQGIGDTIRISLTPEPGADRSLEVIVAQEILQTMGLRSFTPMVISCPGCGRTTSDYFQKLAQQIQTHLRHKMPEWRRRYRGVEDMHVAVMGCVVNGPGESKNANIGISLPGTGEQPVAPVFEDGVKTVTLKGDRIAEEFQELVERYIETHYGSRAEA

SEQ ID NO: 13 - protein, 313 aa, Methylococcus capsulatus
MEIILANPRGFCAGVDRAIEIVDRAIEVFGAPIYVRHEVVHNRYVVDGLRERGAVFVEELSEVPENSTVIFSAHGVSKQIQEEARERGLQVFDATCPLVTKVHIEVHQHASEGREIVFIGHAGHPEVEGTMGQYDNPAGGIYLVESPEDVEMLQVKNPDNLAYVTQTTLSIDDTGAVVEALKMRFPKILGPRKDDICYATQNRQDAVKKLAAQCDTILVVGSPNSSNSNRLREIADKLGRKAFLIDNAAQLTRDMVAGAQRIGVTAGASAPEILVQQVIAQLKEWGGRTATETQGIEEKVVFSLPKELRRLNA

SEQ ID NO: 14 - DNA, 1788 bp, Populus alba
atggccactgaacttctttgtttgcaccgcccgatttccctgacccataagctgtttcgcaaccctctgcccaaagttatccaggcaaccccgctgacgctcaagctccggtgcagcgtatccaccgaaaatgtatcgttcaccgaaaccgaaactgaagcccgtcgcagcgcgaactacgagcccaactcgtgggattacgactatctgctgagctcggataccgacgaatccatcgaagtctataaggacaaagccaagaagctcgaagccgaggtgcgccgtgagatcaacaacgagaaggccgagttcctgaccctgttggaactgatcgacaacgtccagcgcctgggcctcggctaccggttcgagagcgatatccggggtgccctggaccgtttcgtcagctcgggcggattcgacgcagtgaccaaaacgtcgctgcatgggacggccctgtccttccgtctgctgcgccagcatggcttcgaggtgtcccaggaagccttcagcggcttcaaggatcagaacggaaactttctggaaaacttgaaagaggacatcaaggccatcctcagcctgtacgaggcgtccttcctggccctcgaaggtgaaaacatcctcgatgaagccaaggtgttcgcaatctcgcatcttaaagagctgtccgaagagaagattggcaaagagctggccgaacaagtcaaccacgcgttggagctgccgctccaccggcgcacccagcggctggaagcggtctggtcgatcgaagcctaccgcaagaaagaggacgccaatcaggtcctgctggagctcgcgattctggattacaatatgatccagtcggtctatcagcgcgatctgcgcgaaacgtcccggtggtggcggcgtgtcggcttggcgaccaagttgcacttcgcgcgtgaccgcttgatcgagagcttctattgggccgtcggggtggcctttgagccccagtactccgactgccgcaatagcgtggcgaagatgttcagcttcgttaccatcatcgacgacatctacgacgtgtatggcacgctcgacgagctcgaactgttcaccgacgccgtggaacgttgggacgtcaacgccatcaatgatctccccgactacatgaagctgtgcttcctggcgttgtataacaccatcaacgagattgcctacgataacctcaaggacaagggcgagaacatcctgccgtacttgaccaaggcctgggccgatttgtgcaacgcctttctgcaggaagcaaagtggctgtacaacaaatccacgccgacgttcgacgactatttcggcaatgcatggaaatcgagctcgggtcctctgcaacttgtgttcgcgtacttcgccgtcgtgcagaatatcaagaaagaagaaatcgagaaccttcagaaatatcatgacaccatcagccgtccatcgcacatctttcgcctgtgcaacgacctcgcgtccgcatccgccgagatcgcacgcggcgaaacggccaattcggtgtcctgctacatgcggaccaagggcatctcggaagagctggcgacggaatccgtgatgaacctgatcgatgaaacctggaagaagatgaacaaagagaagctcggcgggagcctgttcgcgaagcccttcgtcgaaaccgcaattaacctggcacgccaatcccactgtacctaccataacggagatgcccacacgagcccggacgagctgactcgcaagcgcgtcctttcggtcatcaccgagccgatcctgccgttcgagcggtaa

SEQ ID NO: 15 - DNA, 1680 bp, Populus alba
atgtgcagcgtatccaccgaaaatgtatcgttcaccgaaaccgaaactgaagcccgtcgcagcgcgaactacgagcccaactcgtgggattacgactatctgctgagctcggataccgacgaatccatcgaagtctataaggacaaagccaagaagctcgaagccgaggtgcgccgtgagatcaacaacgagaaggccgagttcctgaccctgttggaactgatcgacaacgtccagcgcctgggcctcggctaccggttcgagagcgatatccggggtgccctggaccgtttcgtcagctcgggcggattcgacgcagtgaccaaaacgtcgctgcatgggacggccctgtccttccgtctgctgcgccagcatggcttcgaggtgtcccaggaagccttcagcggcttcaaggatcagaacggaaactttctggaaaacttgaaagaggacatcaaggccatcctcagcctgtacgaggcgtccttcctggccctcgaaggtgaaaacatcctcgatgaagccaaggtgttcgcaatctcgcatcttaaagagctgtccgaagagaagattggcaaagagctggccgaacaagtcaaccacgcgttggagctgccgctccaccggcgcacccagcggctggaagcggtctggtcgatcgaagcctaccgcaagaaagaggacgccaatcaggtcctgctggagctcgcgattctggattacaatatgatccagtcggtctatcagcgcgatctgcgcgaaacgtcccggtggtggcggcgtgtcggcttggcgaccaagttgcacttcgcgcgtgaccgcttgatcgagagcttctattgggccgtcggggtggcctttgagccccagtactccgactgccgcaatagcgtggcgaagatgttcagcttcgttaccatcatcgacgacatctacgacgtgtatggcacgctcgacgagctcgaactgttcaccgacgccgtggaacgttgggacgtcaacgccatcaatgatctccccgactacatgaagctgtgcttcctggcgttgtataacaccatcaacgagattgcctacgataacctcaaggacaagggcgagaacatcctgccgtacttgaccaaggcctgggccgatttgtgcaacgcctttctgcaggaagcaaagtggctgtacaacaaatccacgccgacgttcgacgactatttcggcaatgcatggaaatcgagctcgggtcctctgcaacttgtgttcgcgtacttcgccgtcgtgcagaatatcaagaaagaagaaatcgagaaccttcagaaatatcatgacaccatcagccgtccatcgcacatctttcgcctgtgcaacgacctcgcgtccgcatccgccgagatcgcacgcggcgaaacggccaattcggtgtcctgctacatgcggaccaagggcatctcggaagagctggcgacggaatccgtgatgaacctgatcgatgaaacctggaagaagatgaacaaagagaagctcggcgggagcctgttcgcgaagcccttcgtcgaaaccgcaattaacctggcacgccaatcccactgtacctaccataacggagatgcccacacgagcccggacgagctgactcgcaagcgcgtcctttcggtcatcaccgagccgatcctgccgttcgagcggtaa

SEQ ID NO: 16 - DNA, 1827 bp, Pueraria montana
atggccaccaatctgctctgcctgtcgaataaactgtccagccccacgcccacgccgtccacgcggttcccgcagtccaagaacttcattacccagaaaaccagcctcgccaacccgaagccatggcgcgtgatctgcgcaacctcgtcccaattcacccagatcacggaacacaactcgcgtcgctcggccaactaccagcctaatttgtggaacttcgagttcctgcagagcttggagaacgatctgaaggtcgagaagctggaagagaaagccaccaagctcgaagaagaggtccgttgcatgatcaaccgcgtcgacactcagccgctctccctgctggagcttatcgacgacgtccagcgcctcggcttgacttacaagttcgagaaagacattatcaaggcccttgagaatatcgtcctgctggatgaaaacaaaaagaacaagtcggatctgcatgcgaccgccctgagcttccggctgctgcgccagcacggctttgaggtcagccaagacgtattcgaacgcttcaaggataaagaaggcgggttttccggcgaattgaaaggcgacgtgcagggcttgctctcgctgtacgaggccagctacctgggctttgagggtgaaaatctgctcgaagaggcgcgtaccttcagcatcacgcatctgaagaataacctcaaagagggcatcaacaccaaggtggccgaacaagtgtcccacgcgctggaactgccataccatcaacggctgcatcgcctggaagcgcgctggttcttggacaagtatgaacccaaagaacctcaccatcagctgcttctggagctcgccaagttggacttcaacatggtccagaccttgcaccagaaagaactgcaggacttgtcccggtggtggaccgaaatgggactggcgtccaagcttgacttcgtccgcgatcgcctcatggaagtgtacttttgggccctcggaatggcaccggacccgcagttcggcgagtgccgcaaagcagttaccaagatgttcggcctggtcaccattatcgacgatgtctacgacgtatacgggacgttggatgagctgcaactgttcacggacgccgtggagcggtgggacgtcaacgccatcaacacgctccccgactatatgaagctctgcttcctggcattgtacaataccgtgaacgacacctcgtattccattctgaaagaaaaaggacacaataacctgtcctatctgaccaagtcctggcgtgagctgtgcaaggcgttcctgcaagaagccaagtggagcaataacaagatcatccccgcgttctcgaagtatcttgagaacgcatccgtgtcgagcagcggggtcgccctgctggccccgtcgtacttcagcgtatgtcagcagcaggaagatatctcggaccacgcgctgcgtagccttacggacttccatggcctcgtccggtcgagctgcgtgatcttccgtttgtgcaacgacctggcgacctcggccgcagaactggagcggggtgaaaccaccaacagcatcatctcgtacatgcacgagaacgatggcacgtcggaagagcaggcacgcgaagagctgcgtaagctgatcgacgccgagtggaagaaaatgaaccgcgaacgcgtcagcgactccaccctgctgccgaaggccttcatggaaatcgccgtgaacatggcacgtgtgtcccattgtacttatcagtacggcgatggcctgggtcgccccgactatgccacggagaaccggatcaagctcctgttgatcgatccgttcccgatcaaccagctgatgtacgtgtaa

SEQ ID NO: 17 - DNA, 1695 bp, Pueraria montana
atgtgcgcaacctcgtcccaattcacccagatcacggaacacaactcgcgtcgctcggccaactaccagcctaatttgtggaacttcgagttcctgcagagcttggagaacgatctgaaggtcgagaagctggaagagaaagccaccaagctcgaagaagaggtccgttgcatgatcaaccgcgtcgacactcagccgctctccctgctggagcttatcgacgacgtccagcgcctcggcttgacttacaagttcgagaaagacattatcaaggcccttgagaatatcgtcctgctggatgaaaacaaaaagaacaagtcggatctgcatgcgaccgccctgagcttccggctgctgcgccagcacggctttgaggtcagccaagacgtattcgaacgcttcaaggataaagaaggcgggttttccggcgaattgaaaggcgacgtgcagggcttgctctcgctgtacgaggccagctacctgggctttgagggtgaaaatctgctcgaagaggcgcgtaccttcagcatcacgcatctgaagaataacctcaaagagggcatcaacaccaaggtggccgaacaagtgtcccacgcgctggaactgccataccatcaacggctgcatcgcctggaagcgcgctggttcttggacaagtatgaacccaaagaacctcaccatcagctgcttctggagctcgccaagttggacttcaacatggtccagaccttgcaccagaaagaactgcaggacttgtcccggtggtggaccgaaatgggactggcgtccaagcttgacttcgtccgcgatcgcctcatggaagtgtacttttgggccctcggaatggcaccggacccgcagttcggcgagtgccgcaaagcagttaccaagatgttcggcctggtcaccattatcgacgatgtctacgacgtatacgggacgttggatgagctgcaactgttcacggacgccgtggagcggtgggacgtcaacgccatcaacacgctccccgactatatgaagctctgcttcctggcattgtacaataccgtgaacgacacctcgtattccattctgaaagaaaaaggacacaataacctgtcctatctgaccaagtcctggcgtgagctgtgcaaggcgttcctgcaagaagccaagtggagcaataacaagatcatccccgcgttctcgaagtatcttgagaacgcatccgtgtcgagcagcggggtcgccctgctggccccgtcgtacttcagcgtatgtcagcagcaggaagatatctcggaccacgcgctgcgtagccttacggacttccatggcctcgtccggtcgagctgcgtgatcttccgtttgtgcaacgacctggcgacctcggccgcagaactggagcggggtgaaaccaccaacagcatcatctcgtacatgcacgagaacgatggcacgtcggaagagcaggcacgcgaagagctgcgtaagctgatcgacgccgagtggaagaaaatgaaccgcgaacgcgtcagcgactccaccctgctgccgaaggccttcatggaaatcgccgtgaacatggcacgtgtgtcccattgtacttatcagtacggcgatggcctgggtcgccccgactatgccacggagaaccggatcaagctcctgttgatcgatccgttcccgatcaaccagctgatgtacgtgtaa

SEQ ID NO: 18 - DNA, 1788 bp, Salix sp.
atggccactgaacttctgtgcttgcaccgtcccatttcgctcacccctaaactgttccgcaacccgctcccgaaggtaatcctggcgacgccgctgaccctgaagctgcggtgcagcgtatccaccgaaaacgtgagctttactgaaaccgaaaccgaaacgcgtcgctcggcgaactacgaacccaattcctgggattatgactaccttctgtcgtccgacacggacgagtcgatcgaggtgtataaggataaggccaagaagcttgaggcggaagtccgtcgggagatcaacaacgagaaggcggagttcctgacgctgctcgaactgattgacaacgtccagcgcctcggcctgggctatcgcttcgagtccgatatccgtcgcgcactcgaccgcttcgtttcgtccggtggcttcgacgcagtgacgaaaacctcgctgcatgccaccgcgctgtcgttccgcttcctgcgccagcacggattcgaggtcagccaggaagcgttcggcgggttcaaggaccagaacgggaatttcctggaaaatctgaaagaagatatcaaagccatcttgtcgctgtacgaggcgtcgtttctcgcgctcgaaggcgagaacattctcgacgaagcgaaggtgttcgccatctcgcacctgaaagagctctccgaagagaagatcggcaaagacttggccgagcaagtcaatcacgccctggagttgcccctgcatcgccgcacccagcgcttggaagccgtttggagcattgaagcctatcgtaagaaagaggacgccaaccaagtcctgctggagctggccatcctggactacaacatgatccagtccgtgtaccagcgggacttgcgcgaaaccagccggtggtggcgtcgcgtcggcctcgccaccaagctgcacttcgcacgcgaccgcctgatcgagtccttctactgggccgtgggcgtcgcattcgagccgcaatatagcgactgccggaacagcgtggcaaagatgttcagcttcgtgaccatcatcgacgatatctatgacgtgtatgggacgcttgacgaactggagctgtttacggatgccgtcgagcggtgggacgtcaatgccatcaacgatttgccggactacatgaagctgtgcttcctggccttgtataacactatcaacgagatcgcctacgataacctgaaagaaaagggtgagaacatcctgccctacctcaccaaggcctgggccgacctgtgtaacgcctttctgcaggaagccaagtggctctacaacaagtccaccccaaccttcgacgattacttcggaaatgcctggaagagcagctccggacctctccagctggtgttcgcatacttcgccgtcgtgcagaacatcaagaaagaagagatcgaaaacttgcagaagtaccacgatatcatcagccgtccctcgcacatcttccggctctgcaacgaccttgcaagcgcgtccgcggagatcgcacggggcgaaacggccaactcggtgagctgctacatgcgcaccaagggcatctcggaagaacttgcgacggagtccgtcatgaacttgatcgacgaaacctggaagaaaatgaataaagagaaactcggcggcagcctgttcccgaagccattcgtcgaaaccgccatcaacctggcgcgtcagtcgcattgcacctaccataatggcgatgcccatacgtcgccggatgaactgacccgtaagcgggtcctgtccgtcatcaccgagccgattctgccgttcgagcgctaa

SEQ ID NO: 19 - DNA, 1680 bp, Salix sp.
atgtgcagcgtatccaccgaaaacgtgagctttactgaaaccgaaaccgaaacgcgtcgctcggcgaactacgaacccaattcctgggattatgactaccttctgtcgtccgacacggacgagtcgatcgaggtgtataaggataaggccaagaagcttgaggcggaagtccgtcgggagatcaacaacgagaaggcggagttcctgacgctgctcgaactgattgacaacgtccagcgcctcggcctgggctatcgcttcgagtccgatatccgtcgcgcactcgaccgcttcgtttcgtccggtggcttcgacgcagtgacgaaaacctcgctgcatgccaccgcgctgtcgttccgcttcctgcgccagcacggattcgaggtcagccaggaagcgttcggcgggttcaaggaccagaacgggaatttcctggaaaatctgaaagaagatatcaaagccatcttgtcgctgtacgaggcgtcgtttctcgcgctcgaaggcgagaacattctcgacgaagcgaaggtgttcgccatctcgcacctgaaagagctctccgaagagaagatcggcaaagacttggccgagcaagtcaatcacgccctggagttgcccctgcatcgccgcacccagcgcttggaagccgtttggagcattgaagcctatcgtaagaaagaggacgccaaccaagtcctgctggagctggccatcctggactacaacatgatccagtccgtgtaccagcgggacttgcgcgaaaccagccggtggtggcgtcgcgtcggcctcgccaccaagctgcacttcgcacgcgaccgcctgatcgagtccttctactgggccgtgggcgtcgcattcgagccgcaatatagcgactgccggaacagcgtggcaaagatgttcagcttcgtgaccatcatcgacgatatctatgacgtgtatgggacgcttgacgaactggagctgtttacggatgccgtcgagcggtgggacgtcaatgccatcaacgatttgccggactacatgaagctgtgcttcctggccttgtataacactatcaacgagatcgcctacgataacctgaaagaaaagggtgagaacatcctgccctacctcaccaaggcctgggccgacctgtgtaacgcctttctgcaggaagccaagtggctctacaacaagtccaccccaaccttcgacgattacttcggaaatgcctggaagagcagctccggacctctccagctggtgttcgcatacttcgccgtcgtgcagaacatcaagaaagaagagatcgaaaacttgcagaagtaccacgatatcatcagccgtccctcgcacatcttccggctctgcaacgaccttgcaagcgcgtccgcggagatcgcacggggcgaaacggccaactcggtgagctgctacatgcgcaccaagggcatctcggaagaacttgcgacggagtccgtcatgaacttgatcgacgaaacctggaagaaaatgaataaagagaaactcggcggcagcctgttcccgaagccattcgtcgaaaccgccatcaacctggcgcgtcagtcgcattgcacctaccataatggcgatgcccatacgtcgccggatgaactgacccgtaagcgggtcctgtccgtcatcaccgagccgattctgccgttcgagcgctaa

(19) United States
(12) Patent Application Publication
(10) Pub. No.: US 2016/0017374 A1
(43) Pub. Date: Jan. 21, 2016

(54) COMPOSITIONS AND METHODS FOR BIOLOGICAL PRODUCTION OF ISOPRENE

(71) Applicant: CALYSTA, INC., Menlo Park, CA (US)

(72) Inventors: Effendi Leonard, Anaheim, CA (US); Jeremy Minshull, Los Altos, CA (US); Jon Edward Ness, Redwood City, CA (US); Thomas Joseph Purcell, Mountain View, CA (US)

(21) Appl. No.: 14/773,118

(22) PCT Filed: Mar. 6, 2014
(86) PCT No.: PCT/US2014/021258

§ 371 Date: Sep. 4, 2015

Related U.S. Application Data
(60) Provisional application No. 61/774,342, filed on Mar. 7, 2013.
(60) Provisional application No. 61/928,333, filed on Jan. 16, 2014.

(51) Int. Cl. C12P 5/00; C12N 9/10; C07C 11/10; C12N 9/12; C12N 9/02; C12N 9/88; C12N 9/04

(52) U.S. Cl. CPC C12P 5/007; C12N 9/88; C12N 9/1022; C12N 9/0006; C12N 9/1205; C12N 9/0093; C12N 9/1241; C07C 11/10; C12Y 101/01267; C12Y 117/01002; C12Y 117/07001; C12Y 207/01148; C12Y 207/0706; C12Y 402/03027; C12Y 406/01012

(57) ABSTRACT

The present disclosure provides compositions and methods for biologically producing isoprene using methanotrophic bacteria that utilize carbon feedstock, such as methane or natural gas.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 200206_{—}411 WO_SEQUENCE_LISTING.txt. The text file is 58.5 KB, was created on Mar. 4, 2014, and is being submitted electronically via EFS-Web.

### BACKGROUND

1. Technical Field

The present disclosure provides compositions and methods for biologically producing isoprene, and more specifically, using methanotrophic bacteria to produce isoprene from carbon substrates, such as methane or natural gas.

2. Description of the Related Art

Isoprene, also known as 2-methyl-1,3-butadiene, is a volatile 5-carbon hydrocarbon. Isoprene is produced by a variety of organisms, including microbes, plants, and animal species (Kuzuyama, 2002, Biosci Biotechnol. Biochem. 66:1619-1627). There are two pathways for isoprene biosynthesis: the mevalonate (MVA) pathway and the non-mevalonate (or mevalonate-independent) pathway, also known as the 1-deoxy-D-xylulose-5-phosphate (DXP) pathway. The MVA pathway is present in eukaryotes, archaea, and cytosol of higher plants (Kuzuyama, 2002, Biosci. Biotechnol. Biochem. 66:1619-1627). The DXP pathway is found in most bacteria, green algae, and the chloroplasts of higher plants (Kuzuyama, 2002, Biosci Biotechnol. Biochem. 66:1619-1627).

Isoprene is an important platform chemical for the production of polyisoprene, for use in the tire and rubber industry; elastomers, for use in footwear, medical supplies, latex, sporting goods; adhesives; and isoprenoids for medicines. Isoprene may also be utilized as an alternative fuel. Isoprene can be chemically modified using catalysts into dimer (10-carbon) and trimer (15-carbon) hydrocarbons to make alkenes (Clement et al., 2008, Chem. Eur. J. 14:7408-7420; Gordillo et al., 2009, Adv. Synth. Catal. 351:325-330). These molecules after being hydrogenated to make long-chain, branched alkanes, may be suitable for use as a diesel or jet fuel replacement.

Currently, isoprene's industrial use is limited by its tight supply. Most synthetic rubbers are based on butadiene polymers, which is substantially more toxic than isoprene. Natural rubber is obtained from rubber trees or plants from Central and South American and African rainforests. Isoprene may also be prepared from petroleum, most commonly by cracking hydrocarbons present in the naphtha portion of refined petroleum. About seven gallons of crude oil are required to make a gallon of fossil-based isoprene. The isoprene yields from naturally producing organisms are not commercially attractive.

Increasing efforts have been made to enable or enhance microbial production of isoprene from abundant and cost-effective renewable resources. In particular, recombinant microorganisms, such as E. coli, algae, and cyanobacteria, have been used to convert biomass-derived feedstocks to isoprene. However, even with the use of relatively inexpensive cellulosic biomass as feedstock, more than half the mass of carbohydrate feedstocks is comprised of oxygen, which represents a significant limitation in conversion efficiency. Isoprene and its derivatives (such as isoprenoids) have significantly lower oxygen content than the feedstocks, which limits the theoretical yield as oxygen must be eliminated as waste. Thus, the economics of production of isoprene and its derivatives from carbohydrate feedstocks is prohibitively expensive.

In view of the limitations associated with carbohydrate-based fermentation methods for production of isoprene and related compounds, there is a need in the art for alternative, cost-effective, and environmentally friendly methods for producing isoprene. The present disclosure meets such needs, and further provides other related advantages.

### BRIEF SUMMARY

In brief, the present disclosure provides for non-naturally occurring methanotrophic bacteria comprising an exogenous nucleic acid encoding an isoprene synthase (e.g., IspS), wherein the methanotrophic bacteria are capable of converting a carbon feedstock into isoprene.

A nucleic acid encoding isoprene synthase may be derived from any organism that contains an endogenous isoprene synthase, such as Populus alba, Populus trichocarpa, Populus tremuloides, Populus nigra, Populus alba×Populus tremula, Populus×canescens, Pueraria montana, Pueraria lobata, Quercus robur, Faboideae, Salix discolor, Salix glabra, Salix pentandra, or Salix serpyllifolia. The exogenous nucleic acid encoding IspS may further be codon optimized for expression in the methanotrophic bacteria. The isoprene synthase may further comprise an amino acid sequence comprising any one of SEQ ID NOs:1-6. The isoprene synthase may also not include an N-terminal plastid targeting sequence. The nucleic acid encoding isoprene synthase may further comprise any one of SEQ ID NOs:14-19.

An exogenous nucleic acid encoding isoprene synthase may further be operatively linked to an expression control sequence. The expression control sequence may further be a promoter selected from the group consisting of methanol dehydrogenase promoter, hexulose-6-phosphate synthase promoter, ribosomal protein S16 promoter, serine hydroxymethyl transferase promoter, serine-glyoxylate aminotransferase promoter, phosphoenolpyruvate carboxylase promoter, T5 promoter, and Trc promoter.

The non-naturally occurring methanotrophic bacteria may further include methanotrophic bacteria that overexpress an endogenous DXP pathway enzyme as compared to the normal expression level of the endogenous DXP pathway enzyme, are transformed with an exogenous nucleic acid encoding a DXP pathway enzyme, or a combination thereof. The DXP pathway enzyme may be DXS, DXR, IDI, IspD, IspE, IspF, IspG, IspH, or a combination thereof. The non-naturally occurring methanotrophic bacteria may further include methanotrophic bacteria that express a transformed exogenous nucleic acid encoding a mevalonate pathway enzyme. The mevalonate pathway enzyme may be acetoacetyl-CoA thiolase, 3-hydroxy-3-methylglutaryl-CoA synthase, 3-hydroxy-3-methylglutaryl-CoA reductase, mevalonate kinase, phophomevalonate kinase, mevalonate pyrophosphate decarboxylase, isopentenyl diphosphate isomerase, or a combination thereof. The non-naturally occurring methanotrophic bacteria may further include at least one exogenous nucleic acid encoding a variant DXP pathway enzyme. The variant DXP pathway enzymes may comprise a mutant pyruvate dehydrogenase (PDH) and a mutant 3,4 dihydroxy-2-butanone 4-phosphate synthase (DHBPS).

The methanotrophic bacteria may further produce from about 1 mg/L to about 500 g/L of isoprene.

An exogenous nucleic acid encoding an isoprene synthase may be introduced into methanotrophic bacteria, such as Methylomonas, Methylobacter, Methylococcus, Methylosinus, Methylocystis, Methylomicrobium, Methanomonas, Methylocella, Methylocapsa. In certain embodiments, the methanotrophic bacteria are Methylococcus capsulatus Bath strain, Methylomonas methanica 16a (ATCC PTA 2402), Methylosinus trichosporium OB3b (NRRL B-11, 196), Methylosinus sporium (NRRL B-11, 197), Methylocystis parvus (NRRL B-11, 198), Methylomonas methanica (NRRL B-11, 199), Methylomonas albus (NRRL B-11, 200), Methylobacter capsulatus (NRRL B-11, 201), Methylobacterium organophilum (ATCC 27, 886), Methylomonas sp AJ-3670 (FERM P-2400), Methylocella silvestris, Methylocella palustris (ATCC 700799), Methylocella tundrae, Methylocystis daltona strain SB2, Methylocystis bryophila, Methylocapsa aurea KYG, Methylacidiphilum infernorum, Methylacidiphilum fumariolicum, Methyloacida kamchatkensis, Methylibium petroleiphilum, or Methylomicrobium alcaliphilum.

In certain embodiments, the carbon feedstock is methane, methanol, natural gas or unconventional natural gas.

Also provided herein are methods for producing isoprene, comprising: culturing a non-naturally occurring methanotrophic bacterium comprising an exogenous nucleic acid encoding isoprene synthase in the presence of a carbon feedstock under conditions sufficient to produce isoprene. The methods include use of the various embodiments described for the non-naturally occurring methanotrophic bacteria. In certain embodiments, the methods further comprising recovering the isoprene produced form the fermentation off-gas. The recovered isoprene may be further modified into a dimer (10-carbon) hydrocarbon, a trimer (15-carbon) hydrocarbon, or a combination thereof. The dimer hydrocarbon, trimer hydrocarbon, or combination thereof, may be further hydrogenated into long-chain branched alkanes. In other embodiments, the recovered isoprene may be further modified into an isoprenoid product.

In another aspect, the present disclosure provides methods for screening mutant methanotrophic bacteria comprising: a) exposing the methanotrophic bacteria to a mutagen to produce mutant methanotrophic bacteria; b) transforming the mutant methanotrophic bacteria with exogenous nucleic acids encoding geranylgeranyl diphosphate synthase (GGPPS), phytoene synthase (CRTB), and phytoene dehydrogenase (CRTI); and c) culturing the mutant methanotrophic bacteria from step b) under conditions sufficient for growth; wherein a mutant methanotrophic bacterium that exhibits an increase in red pigmentation as compared to a reference methanotrophic bacterium that has not been exposed to a mutagen and has been transformed with exogenous nucleic acids encoding GGPPS, CRTB, and CRTI indicates that the mutant methanotrophic bacterium with increased red pigmentation exhibits increased isoprene precursor synthesis as compared to the reference methanotrophic bacterium. In certain embodiments, the mutagen is a radiation, a chemical, a plasmid, or a transposon. In certain embodiments, the mutant methanotrophic bacteria with increased red pigmentation or a clonal cell thereof is transformed with an exogenous nucleic acid encoding IspS. In further embodiments, at least one of the nucleic acids encoding GGPPS, CRTB, and CRTI is removed from or inactivated in the mutant methanotrophic bacterium with increased red pigmentation.

In yet another aspect, the present disclosure provides methods for screening isoprene pathway genes in methanotrophic bacteria comprising: a) transforming the methanotrophic bacteria with: i) at least one exogenous nucleic acid encoding an isoprene pathway enzyme; ii) exogenous nucleic acids encoding geranylgeranyl diphosphate synthase (GGPPS), phytoene synthase (CRTB), and phytoene dehydrogenase (CRTI); and b) culturing the methanotrophic bacteria from step a) under conditions sufficient for growth; wherein the transformed methanotrophic bacterium that exhibits an increase in red pigmentation as compared to a reference methanotrophic bacterium that has been transformed with exogenous nucleic acids encoding GGPPS, CRTB, and CRTI and does not contain the at least one exogenous nucleic acid encoding an isoprene pathway enzyme indicates that the at least one exogenous nucleic acid encoding an isoprene pathway enzyme confers increased isoprene precursor synthesis as compared to the reference methanotrophic bacterium. The isoprene pathway enzyme includes a DXP pathway enzyme or a mevalonate pathway enzyme. The at least one exogenous nucleic acid encoding an isoprene pathway enzyme may comprise a heterologous or homologous nucleic acid. The at least one exogenous nucleic acid encoding an isoprene pathway enzyme may be codon optimized for expression in the host methanotrophic bacteria. The homologous nucleic acid may be overexpressed in the methanotrophic bacteria. The at least one exogenous nucleic acid encoding an isoprene pathway enzyme may comprise a non-naturally occurring variant.

The present disclosure also provides an isoprene composition, wherein the isoprene has a δ¹³C distribution ranging from about −30% to about −50%.

### BRIEF DESCRIPTION OF THE DRAWINGS

FIG. 1 shows the 1-deoxy-D-xylulose-5-phosphate (DXP) pathway for isoprene synthesis. Abbreviations used: DXS=1-deoxy-D-xylulose-5-phosphate (DXP) synthase; DXR=1-deoxy-D-xylulose-5-phosphate (DXP) reductoisomerase, also known as IspC; IspD=4-diphosphocytidyl-2-C-methyl-D-erythritol (CDP-ME) synthase; IspE=4-disphophocytidyl-2-C-methyl-D-erythritol (CDP-ME) kinase; IspF=2-C-methyl-D-erythritol 2,4-cyclodiphosphate (ME-cPP) synthase; IspG=1-hydroxy-2-methyl-2-(E)-butenyl 4-diphosphate (HMBPP) synthase; IspH=1-hydroxy-2-methyl-butenyl 4-diphosphate (HMBPP) reductase; IDI=isopentenyl diphosphate (IPP) isomerase, also known as IPI; IspS=isoprene synthase.

FIG. 2 shows the mevalonate (MVA) pathway for isoprene synthesis. Abbreviations used: AACT=acetoacetyl-CoA thiolase; HMGS=hydroxymethylglutaryl-CoA (HMG) synthase; HMGR=hydroxymethylglutaryl-CoA (HMG) reductase; MK=mevalonate (MVA) kinase; PMK=phosphomevalonate kinase; MPD=mevalonate pyrophosphate decarboxylase, also known as disphosphomevalonate decarboxylase (DPMDC); IDI=isopentenyl diphosphate (IPP) isomerase; IspS=isoprene synthase.

FIG. 3 shows by way of example how methanotrophic bacteria as provided in the present disclosure may utilize light alkanes (methane, ethane, propane, butane) for isoprene production by transforming methanotrophs with an exogenous nucleic acid encoding IspS.

FIG. 4 shows the δ¹³C distribution of various carbon sources.

FIG. 5 shows GC/MS chromatograph of headspace samples derived from an enclosed culture of M. capsulatus Bath strain transformed with (A) pMS3 vector; and (B) pMS3 [Pmdh+Salix sp. IspS]. The arrow indicates the peak corresponding to isoprene. Isoprene yield via quantification of the peak area in A is below the detection limit. Isoprene yield in B is about 10 mg/L.

FIG. 6 shows the lower portion of a lycopene pathway which may be transformed into a methanotrophic host bacteria and used to screen mutant bacterial strains for improved production of isoprene precursor metabolites. Abbreviations used: GGPPS=geranylgeranyl diphosphate (GGPP) synthase.

FIGS. 7A and 7B show the amount of isoprene detected by GC/MS chromatograph in headspace samples from an enclosed culture of M. capsulatus Bath strain transformed with an expression vector containing pLacIq-Pueraria montana ispS and grown in the presence or absence of IPTG.

### DETAILED DESCRIPTION

The instant disclosure provides compositions and methods for biosynthesis of isoprene from carbon feedstocks that are found in natural gas, such as light alkanes (methane, ethane, propane, and butane). For example, methanotrophic bacteria are transformed with an exogenous nucleic acid encoding isoprene synthase (e.g., IspS) and cultured with a carbon feedstock (e.g., natural gas) to generate isoprene. The recombinant methanotrophic bacteria and related methods described herein allow for methanotrophic bioconversion of carbon feedstock into isoprene for use in the tire or rubber industry, pharmaceuticals, or use as an alternative fuel.

By way of background, methane, particularly in the form of natural gas, represents a cheap and abundant natural resource. As noted previously, carbohydrate based feedstocks contain more than half of their mass in oxygen, which is a significant limitation in conversion efficiency, as isoprene does not contain any oxygen molecules, and isoprenoids have much lower oxygen content than such feedstocks. A solution for the limitations of the current biosynthetic systems is to utilize methane or other light alkanes in natural gas as the feedstock for conversion. Methane and other light alkanes (e.g., ethane, propane, and butane) from natural gas have no oxygen, allowing for significant improvement in conversion efficiency. Furthermore, natural gas is cheap and abundant in contrast to carbohydrate feedstocks, contributing to improved economics of isoprene production.

In the present disclosure, bioconversion of carbon feedstocks into isoprene is achieved by introducing an exogenous nucleic acid encoding isoprene synthase (e.g., IspS) into host methanotrophic bacteria. Additionally, metabolic engineering of the host methanotrophic bacteria may be used to increase isoprene yield, by overexpressing native or exogenous genes associated with isoprene pathways (e.g., DXS, DXR, IspD, IspE, IspF, IspG, IspH, or IDI) to increase isoprene precursors. Also provided are methods for screening mutant methanotrophic bacteria for increased isoprene precursor production by engineering a lycopene pathway into bacteria to provide a colorimetric readout.

Prior to setting forth this disclosure in more detail, it may be helpful to an understanding thereof to provide definitions of certain terms to be used herein. Additional definitions are set forth throughout this disclosure.

In the present description, the term “about” means±20% of the indicated range, value, or structure, unless otherwise indicated. The term “consisting essentially of” limits the scope of a claim to the specified materials or steps and those that do not materially affect the basic and novel characteristics of the claimed invention. It should be understood that the terms “a” and “an” as used herein refer to “one or more” of the enumerated components. The use of the alternative (e.g., “or”) should be understood to mean either one, both, or any combination thereof of the alternatives. As used herein, the terms “include” and “have” are used synonymously, which terms and variants thereof are intended to be construed as non-limiting. The term “comprise” means the presence of the stated features, integers, steps, or components as referred to in the claims, but that it does not preclude the presence or addition of one or more other features, integers, steps, components, or groups thereof.

As used herein, the term “isoprene”, also known as “2-methyl-1,3-butadiene,” refers to an organic compound with the formula CH₂═C(CH₃)CH═CH₂. Isoprene is a colorless, hydrophobic, volatile liquid produced by a variety plants, microbial, and animal species. Isoprene is a critical starting material for a variety of synthetic polymers, including synthetic rubbers, and may also be used for fuels.

As used herein, the term “isoprene synthesis pathway”, “isoprene biosynthetic pathway” or “isoprene pathway” refers to any biosynthetic pathway for producing isoprene. Isoprene biosynthesis is generally accomplished via two pathways: the mevalonate (MVA) pathway, which is found in eukaryotes, archaea, and cytosol of higher plants, and the non-mevalonate pathway, also known as methyl-erythritol-4-phosphate (MEP) or (1-deoxy-D-xylulose-5-phosphate) DXP pathway, which may be of prokaryotic origin or from plant plastids. An isoprene pathway may also include pathway variants or modifications of known biosynthetic pathways or engineered biosynthetic pathways.

As used herein, the term “isoprenoid” refers to any compound synthesized from or containing isoprene units (five carbon branched chain isoprene structure). Isoprenoids may include terpenes, ginkgolides, sterols, and carotenoids,

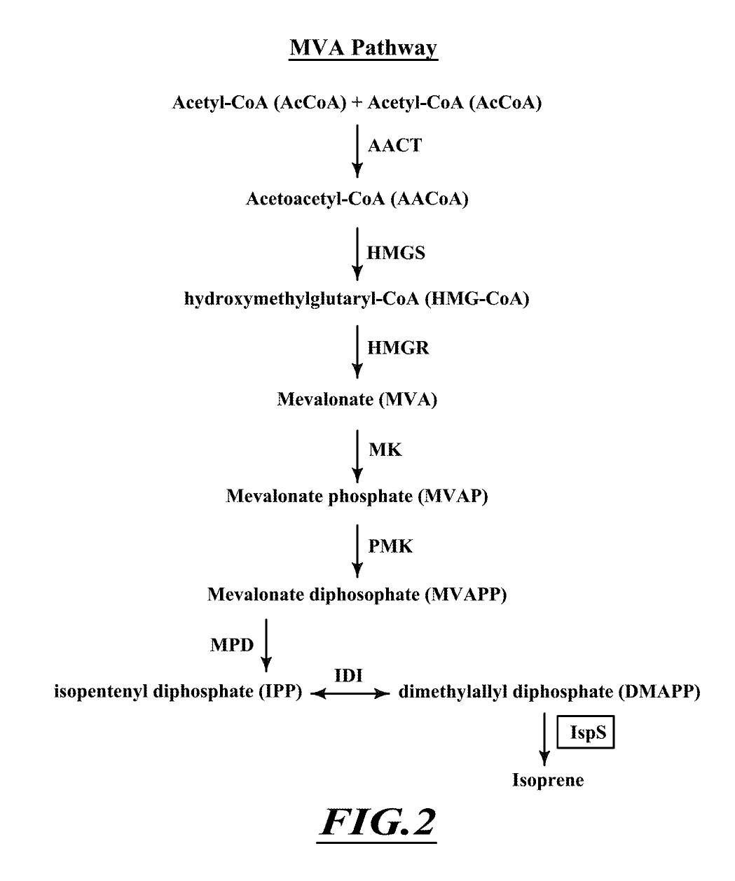

As used herein, the term “mevalonate pathway” or “MVA pathway” refers to an isoprene biosynthetic pathway generally found in eukaryotes and archaea. The mevalonate pathway includes both the classical pathway, as described in FIG. 2, and modified MVA pathways, such as one that converts mevalonate phosphate to isopentenyl phosphate via phophomevalonate decarboxylase (PMDC), which is converted to isopentenyl diphosphate via isopentenyl phosphate kinase (IPK).

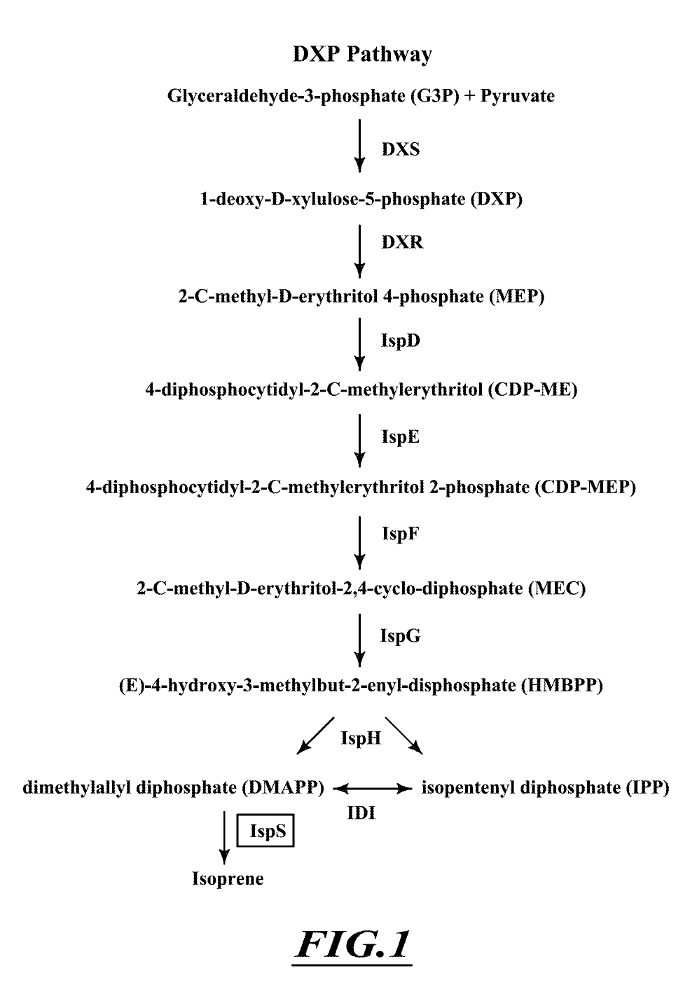

As used herein, the term “non-mevalonate pathway” or “1-deoxy-D-xylulose-5-phosphate (DXP) pathway,” refers to an isoprene biosynthetic pathway generally found in bacteria and plant plastids. An exemplary DXP pathway is shown in FIG. 1.

As used herein, the term “DXP” refers to 1-deoxy-D-xylulose-5-phosphate. 1-deoxy-D-xylulose-5-phosphate synthase (DXS) catalyzes the condensation of glyceraldehydes and pyruvate to form DXP, which is a precursor molecule to isoprene in the DXP pathway.

As used herein, the term “isoprene synthase” (e.g., IspS) refers to an enzyme that catalyzes the conversion of dimethylallyl diphosphate (DMAPP) to isoprene.

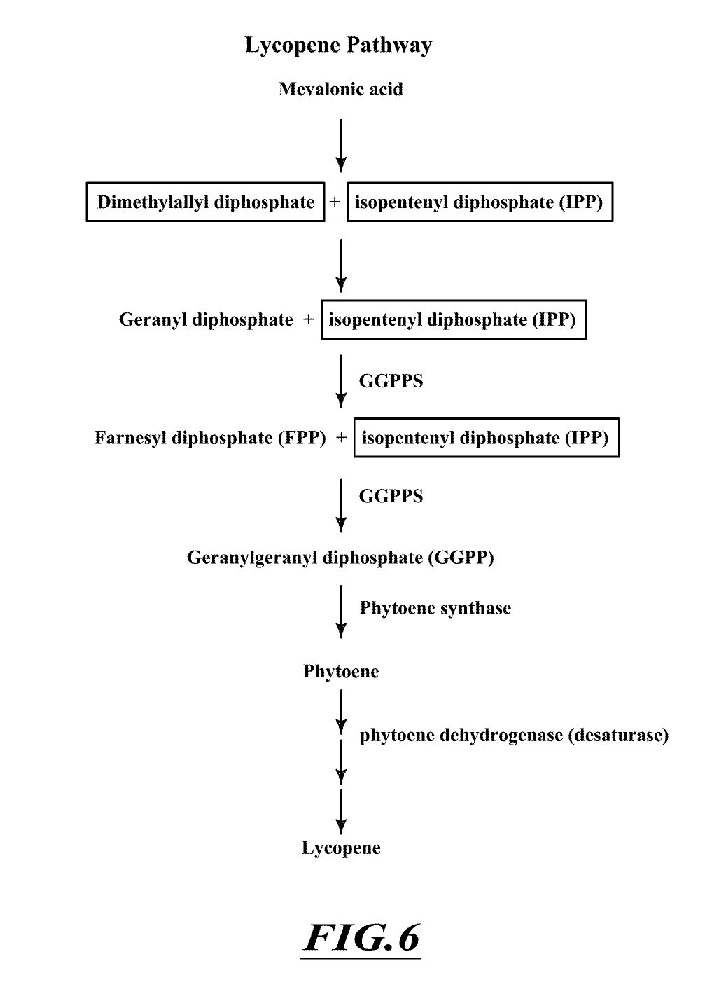

As used herein, the term “lycopene pathway” refers to a biosynthetic pathway for producing lycopene. Lycopene is a bright red carotenoid pigment that is usually found in tomatoes and other red fruits and vegetables. An example of a lycopene pathway is shown in FIG. 6. Generally, lycopene biosynthesis in eukaryotic plants and prokaryotes is similar, beginning with mevalonic acid, which is converted into dimethylallyl diphosphate (DMAPP). Dimethylallyl diphosphate is condensed with three molecules of IPP to produce geranylgeranyl pyrophosphate (GGPP). Two molecules of GGPP are condensed in a tail-to-tail fashion to yield phytoene, which undergoes several desaturation steps to produce lycopene.

As used herein, the term “host” refers to a microorganism (e.g., methanotrophic bacteria) that may be genetically modified with isoprene biosynthetic pathway components (e.g., IspS) to convert a carbon substrate feedstock (e.g., methane, natural, light alkanes) into isoprene. A host cell may contain an endogenous pathway for isoprene precursor synthesis (e.g., DMAPP or IPP) or may be genetically modified to allow or enhance the precursor production. Additionally, a host cell may already possess other genetic modifications that confer desired properties unrelated to the isoprene biosynthesis pathway disclosed herein. For example, a host cell may possess genetic modifications conferring high growth, tolerance of contaminants or particular culture conditions, ability to metabolize additional carbon substrates, or ability to synthesize desirable products or intermediates.

As used herein, the term “methanotroph,” “methanotrophic bacterium” or “methanotrophic bacteria” refers to a methylotrophic bacterium capable of utilizing C₁ substrates, such as methane or unconventional natural gas, as a primary or sole carbon and energy source. As used herein, “methanotrophic bacteria” include “obligate methanotrophic bacteria” that can only utilize C₁ substrates as carbon and energy sources and “facultative methanotrophic bacteria” that are naturally able to use multi-carbon substrates, such as acetate, pyruvate, succinate, malate, or ethanol, in addition to C₁ substrates, as their primary or sole carbon and energy source. Facultative methanotrophs include some species of Methylocella, Methylocystis, and Methylocapsa (e.g., Methylocella silvestris, Methylocella palustris, Methylocella tundrae, Methylocystis daltona SB2, Methylocystis bryophila, and Methylocapsa aurea KYG), and Methylobacterium organophilum (ATCC 27, 886).

As used herein, the term “C1 substrate” or “C1 feedstock” refers to any carbon-containing molecule that lacks a carbon-carbon bond. Examples include methane, methanol, formaldehyde, formic acid, formate, methylated amines (e.g., mono-, di-, and tri-methyl amine), methylated thiols, and carbon dioxide.

As used herein, the term “light alkane” refers to methane, ethane, propane, or butane, or any combination thereof. A light alkane may comprise a substantially purified composition, such as “pipeline quality natural gas” or “dry natural gas”, which is 95-98% methane, or an unpurified composition, such as “wet natural gas”, wherein other hydrocarbons (e.g., ethane, propane, and butane) have not yet been removed and methane comprises more than 60% of the composition. Light alkanes may also be provided as “natural gas liquids”, also known as “natural gas associated hydrocarbons”, which refers to the various hydrocarbons (e.g., ethane, propane, butane) that are separated from wet natural gas during processing to produce pipeline quality dry natural gas. “Partially separated derivative of wet natural gas” includes natural gas liquids.

As used herein, the term “natural gas” refers to a naturally occurring hydrocarbon gas mixture primarily made up of methane, which may have one or more other hydrocarbons (e.g., ethane, propane, and butane), carbon dioxide, nitrogen, and hydrogen sulfide. Natural gas includes conventional natural gas and unconventional natural gas (e.g., tight gas sands, gas shales, gas hydrates, and coal bed methane). Natural gas includes dry natural gas (or pipeline quality natural gas) or wet (unprocessed) natural gas.

As used herein, the term “non-naturally occurring”, also known as “recombinant” or “transgenic”, when used in reference to a microorganism, means that the microorganism has at least one genetic alternation that is not normally found in a naturally occurring strain of the referenced species, including wild-type strains of the referenced species. Genetic alterations include, for example, modifications introducing expressible nucleic acid molecules encoding proteins, other nucleic acid additions, nucleic acid deletions, nucleic acid substitutions, or other functional disruption of the bacterium's genetic material. Such modifications include, for example, coding regions and functional fragments thereof for heterologous or homologous polypeptides for the referenced species. Additional modifications include, for example, non-coding regulatory regions in which the modifications alter expression of a gene or operon. Exemplary proteins include proteins within an isoprene pathway (e.g., IspS). Genetic modifications to nucleic acid molecules encoding enzymes, or functional fragments thereof, can confer a biochemical reaction capability or a metabolic pathway capability or improvements of such capabilities to the non-naturally occurring microorganism that is altered from its naturally occurring state.

As used herein, “exogenous” means that the referenced molecule (e.g., nucleic acid) or referenced activity (e.g., isoprene synthase activity) is introduced into a host microorganism. The molecule can be introduced, for example, by introduction of a nucleic acid into the host genetic material such as by integration into a host chromosome or by introduction of a nucleic acid as non-chromosomal genetic material, such as on a plasmid. When the term is used in reference to expression of an encoding nucleic acid, it refers to introduction of the encoding nucleic acid in an expressible form into the host microorganism. When used in reference to an enzymatic or protein activity, the term refers to an activity that is introduced into the host reference microorganism. Therefore, the term “endogenous” or “native” refers to a referenced molecule or activity that is present in the host microorganism. The term “chimeric” when used in reference to a nucleic acid refers to any nucleic acid that is not endogenous, comprising sequences that are not found together in nature. For example, a chimeric nucleic acid may comprise regulatory sequences and coding sequences that are derived from different sources, or regulatory sequences and coding sequences that are derived from the same source, but arranged in a manner different than that found in nature. The term “heterologous” refers to a molecule or activity that is derived from a source other than the referenced species or strain whereas “homologous” refers to a molecule or activity derived from the host microorganism. Accordingly, a microorganism comprising an exogenous nucleic acid as provided in the present disclosure can utilize either or both a heterologous or homologous nucleic acid.

It is understood that when an exogenous nucleic acid is included in a microorganism that the exogenous nucleic acid refers to the referenced encoding nucleic acid or protein activity, as discussed above. It is also understood that such an exogenous nucleic acid can be introduced into the host microorganism on separate nucleic acid molecules, on a polycistronic nucleic acid molecule, on a single nucleic acid molecule encoding a fusion protein, or a combination thereof, and still be considered as more than one exogenous nucleic acid. For example, as disclosed herein, a microorganism can be modified to express one or more exogenous nucleic acids encoding an enzyme from an isoprene pathway (e.g., isoprene synthase). Where two exogenous nucleic acids encoding enzymes from an isoprene pathway are introduced into a host microorganism, it is understood that the two exogenous nucleic acids can be introduced as a single nucleic acid molecule, for example, on a single plasmid, on separate plasmids, can be integrated into the host chromosome at a single site or multiple sites, and still be considered two exogenous nucleic acids. Similarly, it is understood that more than two exogenous nucleic acid molecules can be introduced into a host microorganism in any desired combination, for example, on a single plasmid, on separate plasmids, can be integrated into the host chromosome at a single site or multiple sites, and still be considered as two or more exogenous nucleic acids. Thus, the number of referenced exogenous nucleic acids or enzymatic activities refers to the number of encoding nucleic acids or the number of protein activities, not the number of separate nucleic acid molecules introduced into the host microorganism.

As used herein, “nucleic acid”, also known as polynucleotide, refers to a polymeric compound comprised of covalently linked subunits called nucleotides. Nucleic acids include polyribonucleic acid (RNA), polydeoxyribonucleic acid (DNA), both of which may be single or double stranded. DNA includes cDNA, genomic DNA, synthetic DNA, and semi-synthetic DNA.

As used herein, “overexpressed” when used in reference to a gene or a protein refers to an increase in expression or activity of the gene or protein. Increased expression or activity includes when the expression or activity or a gene or protein is increased above the level of that in a wild-type (non-genetically engineered) control or reference microorganism. A gene or protein is overexpressed if the expression or activity is in a microorganism where it is not normally expressed or active. A gene or protein is overexpressed if the expression or activity is present in the microorganism for a longer period of time than in a wild-type control or reference microorganism.

### Host Methanotrophic Bacteria

Transformation refers to the transfer of a nucleic acid (e.g., exogenous nucleic acid) into the genome of a host microorganism, resulting in genetically stable inheritance. Host microorganisms containing the transformed nucleic acid are referred to as “non-naturally occurring” or “recombinant” or “transformed” or “transgenic” microorganisms. Host microorganisms may be selected from, or the non-naturally occurring microorganisms generated from, a methanotrophic bacterium, which generally include bacteria that have the ability to oxidize methane as a carbon and energy source.

Methanotrophic bacteria are classified into three groups based on their carbon assimilation pathways and internal membrane structure: type I (gamma proteobacteria), type II (alpha proteobacteria, and type X (gamma proteobacteria). Type I methanotrophs use the ribulose monophosphate (RuMP) pathway for carbon assimilation whereas type II methanotrophs use the serine pathway. Type X methanotrophs use the RuMP pathway but also express low levels of enzymes of the serine pathway. Methanotrophic bacteria are grouped into several genera: Methylomonas, Methylobacter, Methylococcus, Methylocystis, Methylosinus, Methylomicrobium, Methanomonas, and Methylocella.

Methanotrophic bacteria include obligate methanotrophs and facultative methanotrophs, which naturally have the ability to utilize some multi-carbon substrates as a sole carbon and energy source. Facultative methanotrophs include some species of Methylocella, Methylocystis, and Methylocapsa (e.g., Methylocella silvestris, Methylocella palustris, Methylocella tundrae, Methylocystis daltona strain SB2, Methylocystis bryophila, and Methylocapsa aurea KYG). Exemplary methanotrophic bacteria species include: Methylococcus capsulatus Bath strain, Methylomonas 16a (ATCC PTA 2402), Methylosinus trichosporium OB3b (NRRL B-11, 196), Methylosinus sporium (NRRL B-11, 197), Methylocystis parvus (NRRL B-11, 198), Methylomonas methanica (NRRL B-11, 199), Methylomonas albus (NRRL B-11, 200), Methylobacter capsulatus (NRRL B-11, 201), Methylobacterium organophilum (ATCC 27, 886), Methylomonas sp AJ-3670 (FERM P-2400), Methylocella silvestris, Methylocella palustris (ATCC 700799), Methylocella tundrae, Methylocystis daltona strain SB2, Methylocystis bryophila, Methylocapsa aurea KYG, Methylacidiphilum infernorum, Methylacidiphilum fumariolicum, Methyloacida kamchatkensis, Methylibium petroleiphilum, and Methylomicrobium alcaliphilum.

A selected methanotrophic host bacteria may also be subjected to strain adaptation under selective conditions to identify variants with improved properties for production. Improved properties may include increased growth rate, yield of desired products, and tolerance of likely process contaminants (see, e.g., U.S. Pat. No. 6,689,601). In particular embodiments, a high growth variant methanotrophic bacteria is an organism capable of growth on methane as the sole carbon and energy source and possesses an exponential phase growth rate that is faster (i.e., shorter doubling time) than its parent, reference, or wild-type bacteria.

### Isoprene Synthesis Pathways, Nucleic Acids, and Polypeptides

The present disclosure provides methanotrophic bacteria that have been engineered with the capability to produce isoprene. The enzymes comprising the upper portion of the DXP pathway are present in many methanotrophic bacteria. However, following conversion of (E)-4-hydroxy-3-methylbut-2-enyl-diphosphate (HMBPP) into isoprentenyl diphosphate (IPP) and dimethylallyl dipshophate (DMAPP), currently known methanotrophs lack an isoprene synthase (e.g., IspS) for converting DMAPP into isoprene. Instead, methanotrophs convert DMAPP into farnesyl diphosphate via geranyl transferase and farnesyl disphosphate synthase (IspA), which is then converted into carotenoids (see, e.g., U.S. Pat. No. 7,105,634).

In certain embodiments, the present disclosure provides non-naturally occurring methanotrophic bacteria comprising an exogenous nucleic acid encoding an isoprene synthase (e.g., IspS), wherein the methanotrophic bacteria are capable of converting a carbon feedstock into isoprene. Methanotrophic bacteria transformed with an exogenous nucleic acid encoding isoprene synthase are generally capable of converting pyruvate and glyceraldehyde-3-phosphate into isoprene using the DXP pathway as shown in FIG. 1.

Isoprene synthase nucleic acid and polypeptide sequences are known in the art and may be obtained from any organism that naturally possesses isoprene synthase. IspS genes have been isolated and cloned from a number of plants, including for example, poplar, aspen, and kudzu. While a number of bacteria possess DXP pathways, no sequences of the ispS gene from prokaryotes are available in any databases at present (see, e.g., Xue et al., 2011, Appl. Environ. Microbiol. 77:2399-2405). In certain embodiments, a nucleic acid encoding an isoprene synthase is derived from Populus alba, Populus trichocarpa, Populus tremuloides, Populus nigra, Populus alba×Populus tremula, Populus×canescens, Pueraria montana, Pueraria lobata, Quercus robur, Faboideae, Salix discolor, Salix glabra, Salix pentandra, or Salix serpyllifolia. Examples of nucleic acid sequences for isoprene synthase available in the NCBI database include: Accession Nos. AB198180 (Populus alba), AY341431 (Populus tremuloides), AJ294819 (Populus alba×Populus tremula), AY316691 (Pueraria Montana var. lobata), HQ684728 (Populus nigra), and EU693027 (Populus trichocarpa). Examples of isoprene synthase polypeptides are provided in Table 1. The underlined sequence represents N-terminal plastid targeting sequence that is removed in the truncated versions. In certain embodiments, the exogenous nucleic acid encodes an isoprene synthase polypeptide with an amino acid sequence as set forth in any one of SEQ ID NOs:1-6.

TABLE 1
Examples of Isoprene Synthase Polypeptides

| | | SEQ ID |
|---|---|---|
| Species | Amino Acid sequence | NO. |
| Populus alba | MATELLCLHRPISLTHKLFRNPLPKVIQATPLTLKLRC | 1 |
| | SVSTENVSFTETETEARRSANYEPNSWDYDYLLSSDTD | |
| | ESIEVYKDKAKKLEAEVRREINNEKAEFLTLLELIDNV | |
| | QRLGLGYRFESDIRGALDRFVSSGGFDAVTKTSLHGTA | |
| | LSFRLLRQHGFEVSQEAFSGFKDQNGNFLENLKEDIKA | |
| | ILSLYEASFLALEGENILDEAKVFAISHLKELSEEKIG | |
| | KELAEQVNHALELPLHRRTQRLEAVWSIEAYRKKEDAN | |
| | QVLLELAILDYNMIQSVYQRDLRETSRWWRRVGLATKL | |
| | HFARDRLIESFYWAVGVAFEPQYSDCRNSVAKMFSFVT | |
| | IIDDIYDVYGTLDELELFTDAVERWDVNAINDLPDYMK | |
| | LCFLALYNTINEIAYDNLKDKGENILPYLTKAWADLCN | |
| | AFLQEAKWLYNKSTPTFDDYFGNAWKSSSGPLQLVFAY | |
| | FAVVQNIKKEEIENLQKYHDTISRPSHIFRLCNDLASA | |
| | SAEIARGETANSVSCYMRTKGISEELATESVMNLIDET | |
| | WKKMNKEKLGGSLFAKPFVETAINLARQSHCTYHNGDA | |
| | HTSPDELTRKRVLSVITEPILPFER | |
| Populus alba | MCSVSTENVSFTETETEARRSANYEPNSWDYDYLLSSD | 2 |
| (truncated) | TDESIEVYKDKAKKLEAEVRREINNEKAEFLTLLELID | |
| | NVQRLGLGYRFESDIRGALDRFVSSGGFDAVTKTSLHG | |
| | TALSFRLLRQHGFEVSQEAFSGFKDQNGNFLENLKEDI | |
| | KAILSLYEASFLALEGENILDEAKVFAISHLKELSEEK | |
| | IGKELAEQVNHALELPLHRRTQRLEAVWSIEAYRKKED | |
| | ANQVLLELAILDYNMIQSVYQRDLRETSRWWRRVGLAT | |
| | KLHFARDRLIESFYWAVGVAFEPQYSDCRNSVAKMFSF | |
| | VTIIDDIYDVYGTLDELELFTDAVERWDVNAINDLPDY | |
| | MKLCFLALYNTINEIAYDNLKDKGENILPYLTKAWADL | |
| | CNAFLQEAKWLYNKSTPTFDDYFGNAWKSSSGPLQLVF | |
| | AYFAVVQNIKKEEIENLQKYHDTISRPSHIFRLCNDLA | |
| | SASAEIARGETANSVSCYMRTKGISEELATESVMNLID | |
| | ETWKKMNKEKLGGSLFAKPFVETAINLARQSHCTYHNG | |
| | DAHTSPDELTRKRVLSVITEPILPFER | |
| Pueraria | MATNLLCLSNKLSSPTPTPSTRFPQSKNFITQKTSLAN | 3 |
| montana var. | PKPWRVICATSSQFTQITEHNSRRSANYQPNLWNFEFL | |
| lobata | QSLENDLKVEKLEEKATKLEEEVRCMINRVDTQPLSLL | |
| | ELIDDVQRLGLTYKFEKDIIKALENIVLLDENKKNKSD | |
| | LHATALSFRLLRQHGFEVSQDVFERFKD | |
| | KEGGFSGELKGDVQGLLSLYEASYLGFEGENLLEEART | |
| | FSITHLKNNLKEGINTKVAEQVSHALELPYHQRLHRLE | |
| | ARWFLDKYEPKEPHHQLLLELAKLDFNMVQTLHQKELQ | |
| | DLSRWWTEMGLASKLDFVRDRLMEVYFWALGMAPDPQF | |
| | GECRKAVTKMFGLVTIIDDVYDVYGTLDELQLFTDAVE | |
| | RWDVNAINTLPDYMKLCFLALYNTVNDTSYSILKEKGH | |
| | NNLSYLTKSWRELCKAFLQEAKWSNNKIIPAFSKYLEN | |
| | ASVSSSGVALLAPSYFSVCQQQEDISDHALRSLTDFHG | |
| | LVRSSCVIFRLCNDLATSAAELERGETTNSIISYMHEN | |
| | DGTSEEQAREELRKLIDAEWKKMNRERVSDSTLLPKAF | |
| | MEIAVNMARVSHCTYQYGDGLGRPDYATENRIKLLLID | |
| | PFPINQLMYV | |
| Pueraria | MCATSSQFTQITEHNSRRSANYQPNLWNFEFLQSLEND | 4 |
| montana var. | LKVEKLEEKATKLEEEVRCMINRVDTQPLSLLELIDDV | |
| lobata | QRLGLTYKFEKDIIKALENIVLLDENKKNKSDLHATAL | |
| (truncated) | SFRLLRQHGFEVSQDVFERFKDKEGGFSGELKGDVQGL | |
| | LSLYEASYLGFEGENLLEEARTFSITHLKNNLKEGINT | |
| | KVAEQVSHALELPYHQRLHRLEARWFLDKYEPKEPHHQ | |
| | LLLELAKLDFNMVQTLHQKELQDLSRWWTEMGLASKLD | |
| | FVRDRLMEVYFWALGMAPDPQFGECRKAVTKMFGLVTI | |
| | IDDVYDVYGTLDELQLFTDAVERWDVNAINTLPDYMKL | |
| | CFLALYNTVNDTSYSILKEKGHNNLSYLTKSWRELCKA | |
| | FLQEAKWSNNKIIPAFSKYLENASVSSSGVALLAPSYF | |
| | SVCQQQEDISDHALRSLTDFHGLVRSSCVIFRLCNDLA | |
| | TSAAELERGETTNSIISYMHENDGTSEEQAREELRKLI | |
| | DAEWKKMNRERVSDSTLLPKAFMEIAVNMARVSHCTYQ | |
| | YGDGLGRPDYATENRIKLLLIDPFPINQLMYV | |
| Salix sp. DG- | MATELLCLHRPISLTPKLFRNPLPKVILATPLTLKLRC | 5 |
| 2011 | SVSTENVSFTETETETRRSANYEPNSWDYDYLLSSDTD | |
| | ESIEVYKDKAKKLEAEVRREINNEKAEFLTLLELIDNV | |
| | QRLGLGYRFESDIRRALDRFVSSGGFDAVTKTSLHATA | |
| | LSFRFLRQHGFEVSQEAFGGFKDQNGNFLENLKEDIKA | |
| | ILSLYEASFLALEGENILDEAKVFAISHLKELSEEKIG | |
| | KDLAEQVNHALELPLHRRTQRLEAVWSIEAYRKKEDAN | |
| | QVLLELAILDYNMIQSVYQRDLRETSRWWRRVGLATKL | |
| | HFARDRLIESFYWAVGVAFEPQYSDCRNSVAKMFSFVT | |
| | IIDDIYDVYGTLDELELFTDAVERWDVNAINDLPDYMK | |
| | LCFLALYNTINEIAYDNLKEKGENILPYLTKAWADLCN | |
| | AFLQEAKWLYNKSTPTFDDYFGNAWKSSSGPLQLVFAY | |
| | FAVVQNIKKEEIENLQKYHDIISRPSHIFRLCNDLASA | |
| | SAEIARGETANSVSCYMRTKGISEELATESVMNLIDET | |
| | WKKMNKEKLGGSLFPKPFVETAINLARQSHCTYHNGDA | |
| | HTSPDELTRKRVLSVITEPILPFER | |
| Salix sp. DG- | MCSVSTENVSFTETETETRRSANYEPNSWDYDYLLSSD | 6 |
| 2011 | TDESIEVYKDKAKKLEAEVRREINNEKAEFLTLLELID | |
| (truncated) | NVQRLGLGYRFESDIRRALDRFVSSGGFDAVTKTSLHA | |
| | TALSFRFLRQHGFEVSQEAFGGFKDQNGNFLENLKEDI | |
| | KAILSLYEASFLALEGENILDEAKVFAISHLKELSEEK | |
| | IGKDLAEQVNHALELPLHRRTQRLEAVWSIEAYRKKED | |
| | ANQVLLELAILDYNMIQSVYQRDLRETSRWWRRVGLAT | |
| | KLHFARDRLIESFYWAVGVAFEPQYSDCRNSVAKMFSF | |
| | VTIIDDIYDVYGTLDELELFTDAVERWDVNAINDLPDY | |
| | MKLCFLALYNTINEIAYDNLKEKGENILPYLTKAWADL | |
| | CNAFLQEAKWLYNKSTPTFDDYFGNAWKSSSGPLQLVF | |
| | AYFAVVQNIKKEEIENLQKYHDIISRPSHIFRLCNDLA | |
| | SASAEIARGETANSVSCYMRTKGISEELATESVMNLID | |
| | ETWKKMNKEKLGGSLFPKPFVETAINLARQSHCTYHNG | |
| | DAHTSPDELTRKRVLSVITEPILPFER | |

Isoprene synthase nucleic acid and polypeptide sequences for use in the compositions and methods described herein include variants with improved solubility, expression, stability, catalytic activity, and turnover rate. For example, U.S. Pat. No. 8,173,410, which is hereby incorporated in its entirety, discloses specific isoprene synthase amino acid substitutions with enhanced solubility, expression and activity.

In certain embodiments, it may be desirable to overexpress endogenous DXP pathway enzymes or introduce exogenous DXP pathway genes into host methanotrophs to augment IPP and DMAPP production and isoprene yields. In certain embodiments, non-naturally occurring methanotrophic bacteria comprising an exogenous nucleic acid encoding isoprene synthase (e.g., IspS) as provided herein, further overexpress an endogenous DXP pathway enzyme as compared to the normal expression level of the endogenous DXP pathway enzyme, are transformed with an exogenous nucleic acid encoding a DXP pathway enzyme, or both. “Endogenous” or “native” refers to a referenced molecule or activity that is present in the host methanotrophic bacteria. In further embodiments, non-naturally occurring methanotrophic bacteria comprising an exogenous nucleic acid encoding isoprene synthase (e.g., IspS) as provided herein overexpress two, three, four, five, six, seven, eight, or more endogenous DXP pathway enzymes as compared to the normal expression level of the two, three, four, five, six, seven, eight or more endogenous DXP pathway enzymes; are transformed with exogenous nucleic acids encoding two, three, four, five, six, seven, eight, or more DXP pathway enzyme; or any combination thereof. Overexpression of endogenous enzymes from the DXP pathway, such as DXS, may enhance isoprene production (Xue and Ahring, 2011, Applied Environ. Microbiol. 77:2399-2405). Without wishing to be bound by theory, it is believed that increasing the amount of DXS increases the flow of carbon through the DXP pathway, leading to increased isoprene production. In certain embodiments, metabolite profiling using liquid chromatography-mass spectrometry is used to identify bottlenecks in isoprene synthesis pathway and enzymes to be overexpressed (see, e.g., Pitera et al., 2007, Metabolic Engineering 9:193-207).

Methods for overexpressing nucleic acids in host organisms are known in the art. Overexpression may be achieved by introducing a copy of a nucleic acid encoding an endogenous DXP pathway enzyme or an exogenous (e.g., heterologous) nucleic acid encoding a DXP pathway enzyme into host methanotrophic bacteria. By way of example, a nucleic acid encoding an endogenous DXS enzyme may be transformed into host methanotrophic bacteria along with an exogenous nucleic acid encoding an isoprene synthase (e.g., IspS), or an exogenous nucleic acid encoding a DXS enzyme derived from a non-host methantrophic species may be transformed into host methanotrophic bacteria along with an exogenous nucleic acid encoding an isoprene synthase (e.g., IspS). Overexpression of endogenous DXP pathway enzymes may also be achieved by replacing endogenous promoters or regulatory regions with promoters or regulatory regions that result in enhanced transcription.

In certain embodiments, a DXP pathway enzyme that is overexpressed in host methanotrophic bacteria is DXS, DXR, IDI, IspD, IspE, IspF, IspG, IspH, or a combination thereof. In some embodiments, a DXP pathway enzyme that is overexpressed in host methanotrophic is DXS, IDI, IspD, IspF, or a combination thereof.

Sources of DXP pathway enzymes are known in the art and may be from any organism that naturally possesses a DXP pathway, including a wide variety of plant and bacterial species. For example, DXP pathway enzymes may be found in Bacillus anthracis, Helicobacter pylori, Yersinia pestis, Mycobacterium tuberculosis, Plasmodium falciparum, Mycobacterium marinum, Bacillus subtilis, Escherichia coli, Aquifex aeolicus, Chlamydia muridarum, Campylobacter jejuni, Chlamydia trachomatis, Chlamydophila pneumoniae, Haemophilus influenzae, Neisseria meningitidis, Synechocystis, Methylacidiphilum infernorum V4, Methylocystis sp. SC2, Methylomonas strain 16A, Methylococcus capsulatus Bath strain, some unicellular algae, including Scenedesmus oliquus, and in the plastids of most plant species, including, Arabidopsis thaliana, Populus alba, Populus trichocarpa, Populus tremuloides, Populus nigra, Populus alba×Populus tremula, Populus×canescens, Pueraria montana, Pueraria lobata, Quercus robur, Faboideae, Salix discolor, Salix glabra, Salix pentandra, or Salix serpyllifolia.

Examples of nucleic acid sequences for DXS available in the NCBI database include Accession Nos: AF035440, (Escherichia coli); Y18874 (Synechococcus PCC6301); AB026631 (Streptomyces sp. CL190); AB042821 (Streptomyces griseolosporeus); AF11814 (Plasmodium falciparum); AF143812 (Lycopersicon esculentum); AJ279019 (Narcissus pseudonarcissus); AJ291721 (Nicotiana tabacum); AX398484.1 (Methylomonas strain 16A); NC 010794.1 (region 1435594.1437486, complement) (Methylacidiphilum infernorum V4); and NC 018485.1 (region 2374620.2376548) (Methylocystis sp. SC2).

Examples of nucleic acid sequences for DXR available in the NCBI database include Accession Nos: AB013300 (Escherichia coli); AB049187 (Strepomyces griseolosporeus); AF111813 (Plasmodium falciparum); AF116825 (Mentha×piperita); AF148852 (Arabidopsis thaliana); AF182287 (Artemisia annua); AF250235 (Catharanthus roseus); AF282879 (Pseudomonas aeruginosa); AJ242588 (Arabidopsis thaliana); AJ250714 (Zymomonas mobilis strain ZM4); AJ292312 (Klebsiella penumoniae); AJ297566 (Zea mays); and AX398486.1 (Methylomonas strain 16A). Examples of nucleic acid sequences for IspD available in the NCBI database include Accession Nos: AB037876 (Arabidopsis thaliana); AF109075 (Clostridium difficile); AF230736 (E. coli); AF230737 (Arabidopsis thaliana); and AX398490.1 (Methylomonas strain 16A).

Examples of nucleic acid sequences for IspE available in the NCBI database include Accession Nos: AF216300 (Escherichia coli); AF263101 (Lycopersicon esculentum); AF288615 (Arabidopsis thaliana); and AX398496.1 (Methylomonas strain 16A).

Examples of nucleic acid sequences for IspF available in the NCBI database include Accession Nos: AF230738 (Escherichia coli); AB038256 (Escherichia coli); AF250236 (Catharanthus roseus); AF279661 (Plasmodium falciparum); AF321531 (Arabidopsis thaliana); and AX398488.1 (Methylomonas strain 16A).

Examples of nucleic acid sequences for IspG available in the NCBI database include Accession Nos: AY033515 (Escherichia coli) YP_{—}005646 (Thermus thermopilus), and YP_{—}475776.1 (Synechococcus sp.). Examples of nucleic acid sequences for IspH available in the NCBI database include Accession Nos: AY062212 (Escherichia coli), YP_{—}233819.1 (Pseudomonas syringae), and YP_{—}729527.1 (Synechococcus sp.). Examples of nucleic acid sequences for IDI available in the NCBI database include Accession Nos: AF119715 (E. coli), P61615 (Sulfolobus shibatae), and O42641 (Phaffia rhodozyme).

Amino acid sequences for DXP pathway enzymes from Methylococcus capsulatus Bath strain (ATCC 33009) that may be used in various embodiments are provided in Table 2.

TABLE 2
DXP pathway Enzymes of Methylococcus capsulatus Bath strain

| Gene | | SEQ ID |
|---|---|---|
| Name | Amino Acid Sequence | NO |
| DXS | MTETKRYALLEAADHPAALRNLPEDRLPELAEELRGYLLESVS | 7 |
| | RSGGHLAAGLGTVELTIALHYVFNTPEDKLVWDVGHQAYPHKI | |
| | LTGRRARLPTIRKKGGLSAFPNRAESPYDCFGVGHSSTSISAA | |
| | LGMAVAAALERRPIHAVAIIGDGGLTGGMAFEALNHAGTLDAN | |
| | LLIILNDNEMSISPNVGALNNYLAKILSGKFYSSVRESGKHLL | |
| | GRHMPGVWELARRAEEHVKGMVAPGTLFEELGFNYFGPIDGHD | |
| | LDTLITTLRNLRDQKGPRFLHVVTRKGKGYAPAEKDPVAYHGV | |
| | GAFDLDADELPKSKPGTPSYTEVFGQWLCDMAARDRRLLGITP | |
| | AMREGSGLVEFSQRFPDRYFDVGIAEQHAVTFAAGQASEGYKP | |
| | VVAIYSTFLQRAYDQLIHDVALQNLPVLFAIDRAGLVGPDGPT | |
| | HAGSFDLSFMRCIPNMLIMAPSDENECRQMLYTGFIHDGPAAV | |
| | RYPRGRGPGVRPEETMTAFPVGKGEVRLRGKGTAILAFGTPLA | |
| | AALAVGERIGATVANMRFVKPLDE | |
| | ALILELAATHDRIVTVEENAIAGGAGSAVGEFLAAQHCGIPVC | |
| | HIGLKDEFLDQGTREELLAIAGLDQAGIARSIDAFIQATAAAD | |
| | KPRRARGQAKDKH | |
| DXR | MKGICILGSTGSIGVSTLDVLARHPDRYRVVALSANGNVDRLF | 8 |
| | EQCRAHRPRYAAVIRAEAAACLRERLMAAGLGGIEVLAGPEAL | |
| | EQIASLPEVDSVMAAIVGAAGLLPTLAAARAGKDVLLANKEAL | |
| | VMSGPLFMAEVARSGARLLPIDSEHNAVFQCMPAAYRAGSRAV | |
| | GVRRILLTASGGPFLHTPLAELETVTPEQAVAHPNWVMGRKIS | |
| | VDSATMMNKGLEVIEACLLFNAKPDDVQVVVHRQSVIHSMVDY | |
| | VDGTVLAQMGTPDMRIPIAHALAWPDRFESGAESLDLFAVRQL | |
| | NFERPDLARFPCLRLAYEAVGAGGTAPAILNAANETAVAAFLD | |
| | RRLAFTGIPRVIEHCMARVAPNAADAIESVLQADAETRKVAQK | |
| | YIDDLRV | |
| IspD | MSTDARFWIVVPAAGVGKRMGADIPKQYLDVAGKPVLQHTLER | 9 |
| | LLSVRRVTAVMVALGANDEFWPELPCSREPRVLATTGGRERAD | |
| | SVLSALTALAGRAADGDWVLVHDAARLCVTRDDVERLMETLED | |
| | DPVGGILALPVTDTLKTVENGTIQGSADRSRVWRALTPQMFRY | |
| | RALKEALEAAARRGLTVTDEASALELAGLSPRVVEGRPDNIKI | |
| | TRPEDLPLAAFYLERQCFE | |
| IspE | MDRRESSVMKSPSLRLPAPAKLNLTLRITGRRPDGYHDLQTVF | 10 |
| | QFVDVCDWLEFRADASGEIRLQTSLAGVPAERNLIVRAARLLK | |
| | EYAGVAAGADIVLEKNLPMGGGLGGGSSNAATTLVALNRLWDL | |
| | GLDRQTLMNLGLRLGADVPIFVFGEGAWAEGVGERLQVLELPE | |
| | PWYVIVVPPCHVSTAEIFNAPDLTRDNDPITIADFLAGSHQNH | |
| | CLDAVVRRYPVVGEAMCVLGRYSRDVRLTGTGACVYSVHGSEE | |
| | EAKAACDDLSRDWVAIVASGRNLSPLYEALNER | |
| IspF | MFRIGQGYDAHRFKEGDHIVLCGVKIPFGRGFAAHSDGDVALH | 11 |
| | ALCDALLGAAALGDIGRHFPDTDARYKGIDSRVLLREVRQRIA | |
| | SLGYTVGNVDVTVVAQAPRLAAHIQAMRENLAQDLEIPPDCVN | |
| | VKATTTEGMGFEGRGEGISAHAVALLARR | |
| IspG | MMNRKQTVGVRVGSVRIGGGAPIVVQSMTNTDTADVAGTVRQV | 12 |
| | IDLARAGSELVRITVNNEEAAEAVPRIREELDRQGCNVPLVGD | |
| | FHFNGHKLLDKYPACAEALGKFRINPGNVGRGSKRDPQFAQMI | |
| | EFACRYDKPVRIGVNWGSLDQSVLARLLDENARLAEPRPLPEV | |
| | MREAVITSALESAEKAQGLGLPKDRIVLSCKMSGVQELISVYE | |
| | ALSSRCDHALHLGLTEAGMGSKGIVASTAALSVLLQQGIGDTI | |
| | RISLTPEPGADRSLEVIVAQEILQTMGLRSFTPMVISCPGCGR | |
| | TTSDYFQKLAQQIQTHLRHKMPEWRRRYRGVEDMHVAVMGCVV | |
| | NGPGESKNANIGISLPGTGEQPVAPVFEDGVKTVTLKGDRIAE | |
| | EFQELVERYIETHYGSRAEA | |
| IspH | MEIILANPRGFCAGVDRAIEIVDRAIEVFGAPIYVRHEVVHNR | 13 |
| | YVVDGLRERGAVFVEELSEVPENSTVIFSAHGVSKQIQEEARE | |
| | RGLQVFDATCPLVTKVHIEVHQHASEGREIVFIGHAGHPEVEG | |
| | TMGQYDNPAGGIYLVESPEDVEMLQVKNPDNLAYVTQTTLSID | |
| | DTGAVVEALKMRFPKILGPRKDDICYATQNRQDAVKKLAAQCD | |
| | TILVVGSPNSSNSNRLREIADKLGRKAFLIDNAAQLTRDMVAG | |
| | AQRIGVTAGASAPEILVQQVIAQLKEWGGRTATETQGIEEKVV | |
| | FSLPKELRRLNA | |

It is understood by one skilled in the art that the source of each DXP pathway enzyme that is introduced into the host methanotrophic bacteria may be the same, the sources of two or more DXP pathway enzymes introduced into the host methanotrophic bacteria may be the same, or the source of each DXP pathway enzyme introduced into the host methanotrophic bacteria may differ from one another. The source(s) of the DXP pathway enzymes may be the same or differ from the source of IspS. In certain embodiments, hybrid pathways with nucleic acids derived from two or more sources are used to enhance isoprene production (see, e.g., Yang et al., 2012, PLoS ONE 7:e33509).

It may also be desirable to augment isoprene production by increasing synthesis of isoprene precursors DMAPP and IPP via an alternate pathway. By way of example, DMAPP and IPP may also be synthesized via the mevalonate pathway (see FIG. 2). Without wishing to be bound by theory, it is believed that increasing the amount of DMAPP and IPP polypeptides in cells may increase the amount of isoprene produced. At present, an endogenous mevalonate pathway has not yet been identified in the few methanotrophic bacteria that have been fully sequenced. However, a mevalonate pathway has been identified in a few bacterial species. If a mevalonate pathway is not present in a host methanotroph, it may be desirable to introduce the genes necessary for constructing a mevalonate pathway for production of DMAPP and IPP precursors. If a mevalonate pathway is present in a host methanotroph, it may also be desirable to introduce or overexpress certain mevalonate pathway genes to enhance production of DMAPP and IPP. In certain embodiments, non-naturally occurring methantrophic bacteria comprising an exogenous nucleic acid encoding IspS overexpress an endogenous mevalonate pathway enzyme as compared to the normal expression level of the native mevalonate pathway enzyme, express a transformed exogenous nucleic acid encoding a mevalonate pathway enzyme, or a combination thereof. In further embodiments, non-naturally occurring methanotrophic bacteria comprising an exogenous nucleic acid encoding IspS as provided herein overexpress one, two, three, four, five, six or more endogenous mevalonate pathway enzymes as compared to the normal expression level of the respective endogenous mevalonate pathway enzymes; are transformed with exogenous nucleic acids encoding one, two, three, four, five, six, or more mevalonate pathway enzymes; or both.

Engineering of a mevalonate pathway into methanotrophs or enhancing an endogenous mevalonate pathway may enhance isoprene production by increasing the supply of DMAPP and IPP precursors (see, e.g., Martin et al., 2003, Nature Biotechnol. 21:796-802). In certain embodiments, metabolite profiling using liquid chromatography-mass spectrometry is used to identify bottlenecks in isoprene synthesis pathway and enzymes to be overexpressed (see, e.g., Pitera et al., 2007, Metabolic Engineering 9:193-207). Overexpression may be achieved by introducing a nucleic acid encoding an endogenous mevalonate pathway enzyme or an exogenous (i.e., heterologous) nucleic acid encoding a mevalonate pathway enzyme into host methanotrophic bacteria. By way of example, a copy of a nucleic acid encoding an endogenous mevlaonate enzyme may be transformed into host methanotrophic bacteria along with an exogenous nucleic acid encoding IspS or an exogenous nucleic acid encoding mevalonate enzyme derived from a non-host methantrophic species may be transformed into host methanotrophic bacteria along with an exogenous nucleic acid encoding IspS. Overexpression of endogenous mevalonate pathway enzymes may also be achieved by replacing endogenous promoters or regulatory regions with promoters or regulatory regions that result in enhanced transcription. In certain embodiments, a mevalonate pathway enzyme that is overexpressed in host methanotrophic bacteria is AACT, HMGS, HMGR, MK, PMK, MPD, IDI, or a combination thereof. In some embodiments, a mutant HMGS nucleic acid encoding a polypeptide with a Ala110Gly substitution (to increase reaction rate) is introduced into host methanotrophic bacteria (Steussy et al., 2006, Biochem. 45:14407-14).

Sources of mevalonate pathway enzymes are known in the art and may be from any organism that naturally possesses a mevalonate pathway, including a wide variety of plant, animal, fungal, archaea, and bacterial species. For example, mevalonate pathway enzymes may be found in Caldariella acidophilus, Halobacterium cutirubrum, Myxococcus fulvus, Chloroflexus aurantiacus, Saccharomyces cerevisiae, Schizosaccharomyces pombe, Caenorhabditis elegans, Arabidopsis thaliana, Lactobacillus plantarum, Staphylococcus aureus, Staphylococcus carnosus, Staphylococcus haemolyticus, Staphylococcus epidermidis, Streptococcus mutans, Streptococcus pneumoniae, Streptococcus pyogenes, Streptomyces aeriouvifer, Borrelia burgdorferi, Chloropseudomonas ethylica, Myxococcus fulvus, Euglena gracilis, Enterococcus faecalis, Enterococcus faecium, Archaeoglobus fulgidus, Methanobacterium thermoautotrophicum, Methanococcus jannaschii, Homo sapiens, Enterococcus gallinarum, Enterococcus casseliflavus, Listeria grayi, Methanosarcina mazei, Methanococcoides buronii, Lactobacillus sakei, and Streptomyces CL190. It is understood to one skilled in the art that the source of each mevalonate pathway enzymes introduced into the methanotrophic host bacteria may be the same, the sources of two or more mevalonate pathway enzymes may be the same, or the source of each mevalonate pathway enzyme may differ from one another. The source(s) of the mevalonate pathway enzymes may be the same or differ from the source of an isoprene synthase (e.g., IspS). In certain embodiments, hybrid pathways with nucleic acids derived from two or more sources are used to enhance isoprene production (Yang et al., 2012, PLoS ONE 7:e33509).

In certain embodiments, non-naturally occurring methanotrophic bacteria comprising an exogenous nucleic acid encoding IspS may further comprise genetically modified DXP and mevalonate pathways as described herein. For example, non-naturally occurring methanotrophic bacteria as described herein may overexpress an endogenous DXP pathway enzyme as compared to the normal expression level of the endogenous DXP pathway enzyme, express a transformed exogenous nucleic acid encoding a DXP pathway enzyme, or both; and overexpress an endogenous mevalonate pathway enzyme as compared to the normal expression level of the native mevalonate pathway enzyme, express a transformed exogenous nucleic acid encoding a mevalonate pathway enzyme, or both; or any combination thereof. As noted previously, sources of all the DXP and mevalonate pathway enzymes may be the same, sources of some DXP or mevalonate pathway enzymes may be same, or sources of DXP and mevalonate pathway enzymes may all differ from each other.

Non-naturally occurring methanotrophic bacteria of the instant disclosure may also be engineered to comprise variant isoprene biosynthetic pathways or enzymes. Variation in isoprene synthesis pathways may occur at one or more individual steps of a pathway or involve an entirely new pathway. A particular pathway reaction may be catalyzed by different classes of enzymes that may not have sequence, structural or catalytic similarity to known isoprene enzymes. For example, Brucella abortus 2308 contains genes for a DXP pathway, except DXR. Instead, Brucella abortus 2308 uses a DXR-like gene (DRL) to catalyze the formation of 2-C-methyl-D-erythritol-4-phosphate (MEP) from DXP (Sangari et al., 2010, Proc. Natl. Acad. Sci. USA 107:14081-14086). In another example, mutant aceE and ribE genes, encoding catalytic E subunit of pyruvate dehydrogenase and 3,4-dihydroxy-2-butanone 4-phosphate synthase, respectively, have been identified that are each capable of rescuing DXS-defective mutant bacteria and produce DXP via a variant DXP pathway (Perez-Gil et al., 2012, PLoS ONE 7:e43775). In yet another example, various types of isopentenyl disphosphate isomerases have also been identified (Kaneda et al., 2001, Proc. Natl. Acad. Sci. USA 98:932-7; Laupitz et al., 2004, Eur. J. Biochem. 271:2658-69). Alternative isoprene synthesis pathways in addition to DXP and mevalonate pathways may also exist (see, Poliquin et al., 2004, J. Bacteriol. 186:4685-4693; Ershov et al., 2002, J Bacteriol. 184:5045-5051). In certain embodiments, particular pathway reactions are catalyzed by variant or alternative isoprene enzymes, such as DRL, catalytic E subunit of pyruvate dehydrogenase, 3,4-dihydroxy-2-butanone 4-phosphate synthase, a variant isopentenyl disphosphate isomerase, or any combination thereof.

A nucleic acid encoding an isoprene pathway component (e.g., a nucleic acid encoding an isoprene synthase (e.g., IspS)) includes nucleic acids that encode a polypeptide, a polypeptide fragment, a peptide, or a fusion polypeptide that has at least one activity of the encoded isoprene pathway polypeptide (e.g., ability to convert DMAPP into isoprene). Methods known in the art may be used to determine whether a polypeptide has a particular activity by measuring the ability of the polypeptide to convert a substrate into a product (see, e.g., Silver et al., 1995, J. Biol. Chem. 270:13010-13016).

With the complete genome sequence available for hundreds of organisms, the identification of genes encoding an isoprene synthase and other isoprene pathway enzymes in related or distant species, including for example, homologs, orthologs, paralogs, etc., is well known in the art. Accordingly, exogenous nucleic acids encoding an isoprene synthase, DXS, DXR, IDI, etc., described herein with reference to particular nucleic acids from a particular organism can readily include other nucleic acids encoding an isoprene synthase, DXS, DXR, IDI, etc. from other organisms.

Polypeptide sequences and encoding nucleic acids for proteins, protein domains, and fragments thereof described herein, such as an isoprene synthase and other isoprene pathway enzymes, may include naturally and recombinantly engineered variants. A nucleic acid variant refers to a nucleic acid that may contain one or more substitutions, additions, deletions, insertions, or may be or comprise fragment(s) of a reference nucleic acid. A reference nucleic acid refers to a selected wild-type or parent nucleic acid encoding a particular isoprene pathway enzyme (e.g., IspS). A variant nucleic acid may have 40%, 45%, 50%, 55%, 60%, 65%, 70%, 75%, 80%, 85%, 90%, 95%, 96%, 97%, 98%, or 99% sequence identity to a reference nucleic acid, as long as the variant nucleic acid encodes a polypeptide that can still perform its requisite function or biological activity (e.g., for IspS, converting DMAPP to isoprene). A variant polypeptide may have 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity to a reference protein, as long as the variant polypeptide can still perform its requisite function or biological activity (e.g., for IspS, converting DMAPP to isoprene). In certain embodiments, an isoprene synthase (e.g., IspS) that is introduced into non-naturally occurring methanotrophic bacteria as provided herein comprises an amino acid sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identity to an amino acid sequence provided in SEQ ID NOs:1-6. These variants may have improved function and biological activity (e.g., higher enzymatic activity, improved specificity for substrate, or higher turnover rate) than the parent (or wild-type) protein. Due to redundancy in the genetic code, nucleic acid variants may or may not affect amino acid sequence.

A nucleic acid variant may also encode an amino acid sequence comprising one or more conservative substitutions compared to a reference amino acid sequence. A conservative substitution may occur naturally in the polypeptide (e.g., naturally occurring genetic variants) or may be introduced when the polypeptide is recombinantly produced. A conservative substitution is where one amino acid is substituted for another amino acid that has similar properties, such that one skilled in the art would expect that the secondary structure and hydropathic nature of the polypeptide to be substantially unchanged. Amino acid substitutions may generally be made on the basis of similarity in polarity, charge, solubility, hydrophobicity, or the amphipathic nature of the residues, and is known in the art.

Amino acid substitutions, deletions, and additions may be introduced into a polypeptide using well-known and routinely practiced mutagenesis methods (see, e.g., Sambrook et al. Molecular Cloning: A Laboratory Manual, 3d ed., Cold Spring Harbor Laboratory Press, NY 2001). Oligonucleotide-directed site-specific (or segment specific) mutagenesis procedures may be employed to provide an altered polynucleotide that has particular codons altered according to the substitution, deletion, or insertion desired. Deletion or truncation variants of proteins may also be constructed by using convenient restriction endonuclease sites adjacent to the desired deletion. Alternatively, random mutagenesis techniques, such as alanine scanning mutagenesis, error prone polymerase chain reaction mutagenesis, and oligonucleotide-directed mutagenesis may be used to prepare polypeptide variants (see, e.g., Sambrook et al., supra).

Differences between a wild type (or parent or reference) nucleic acid or polypeptide and the variant thereof, may be determined by known methods to determine identity, which are designed to give the greatest match between the sequences tested. Methods to determine sequence identity can be applied from publicly available computer programs. Computer program methods to determine identity between two sequences include, for example, BLASTP, BLASTN (Altschul, S. F. et al., J. Mol. Biol. 215: 403-410 (1990), and FASTA (Pearson and Lipman Proc. Natl. Acad. Sci. USA 85; 2444-2448 (1988) using the default parameters.

Assays for determining whether a polypeptide variant folds into a conformation comparable to the non-variant polypeptide or fragment include, for example, the ability of the protein to react with mono- or polyclonal antibodies that are specific for native or unfolded epitopes, the retention of ligand-binding functions, the retention of enzymatic activity (if applicable), and the sensitivity or resistance of the mutant protein to digestion with proteases (see Sambrook et al., supra). Polypeptides, variants and fragments thereof, can be prepared without altering a biological activity of the resulting protein molecule (i.e., without altering one or more functional activities in a statistically significant or biologically significant manner). For example, such substitutions are generally made by interchanging an amino acid with another amino acid that is included within the same group, such as the group of polar residues, charged residues, hydrophobic residues, or small residues, or the like. The effect of any amino acid substitution may be determined empirically merely by testing the resulting modified protein for the ability to function in a biological assay, or to bind to a cognate ligand or target molecule.

In certain embodiments, an exogenous nucleic acid encoding IspS or other isoprene pathway enzymes introduced into host methanotrophic bacteria does not comprise an N-terminal plastid-targeting sequence. Generally, chloroplastic proteins, such as many plant isoprene synthases and other isoprene pathway enzymes, are encoded in the nucleus and synthesized in the cytosol as precursors. N-terminal plastid-targeting sequences, also known as a signal peptide or transit peptide, encode a signal required for targeting to chloroplastic envelopes, which is cleaved off by a peptidase after chloroplast import. Removal of N-terminal targeting sequences may enhance expression of heterologous nucleic acids. N-terminal plastid-targeting sequences may be determined using prediction programs known in the art, including ChloroP (Emannuelsson et al., 1999, Protein Sci. 8:978-984); PLCR (Schein et al., 2001, Nucleic Acids Res. 29:e82); MultiP (http://sbi.postech.ac.kr/MultiP/). N-terminal plastid targeting sequences may be removed from nucleic acids by recombinant means prior to introduction into methanotrophic bacteria. In certain embodiments, an amino acid sequence for IspS lacking the N-terminal plastid targeting sequence is provided in any one of SEQ ID NOs:2, 4, and 6. In other embodiments, an exogenous nucleic acid encoding an isoprene synthase (e.g., IspS) or other isoprene pathway enzyme introduced into host methanotrophic bacteria does not include a targeting sequence to other organelles, for example, the apicoplast or endoplasmic reticulum.

In certain embodiments, an exogenous nucleic acid encoding isoprene synthase or other isoprene pathway enzymes is operatively linked to an expression control sequence. An expression control sequence means a nucleic acid sequence that directs transcription of a nucleic acid to which it is operatively linked. An expression control sequence includes a promoter (e.g., constitutive, leaky, or inducible) or an enhancer. In certain embodiments, the expression control sequence is a promoter selected from the group consisting of: methanol dehydrogenase promoter (MDH), hexulose 6-phosphate synthase promoter, ribosomal protein S16 promoter, serine hydroxymethyl transferase promoter, serine-glyoxylate aminotransferase promoter, phosphoenolpyruvate carboxylase promoter, T5 promoter, and Trc promoter. Without wishing to be bound by theory, methanol dehydrogenase promoter, hexulose 6-phosphate synthase promoter, ribosomal protein S16 promoter, serine hydroxymethyl transferase promoter, serine-glyoxylate aminotransferase promoter, phosphoenolpyruvate carboxylase promoter, T5 promoter, and Trc promoter offer varying strengths of promoters that allow expression of heterologous polypeptides in methanotrophic bacteria.

In certain embodiments, a nucleic acid encoding IspS is operatively linked to an inducible promoter. Inducible promoter systems are known in the art and include tetracycline inducible promoter system; IPTG/lac operon inducible promoter system, heat shock inducible promoter system; metal-responsive promoter systems; nitrate inducible promoter system; light inducible promoter system; ecdysone inducible promoter system, etc. For example, a non-naturally occurring methanotroph may comprise an exogenous nucleic acid encoding an isoprene synthase (e.g., IspS), operatively linked to a promoter flanked by lacO operator sequences, and also comprise an exogenous nucleic acid encoding a lad repressor protein operatively linked to a constitutive promoter (e.g., hexulose-6-phosphate synthase promoter). Lad repressor protein binds to lacO operator sequences flanking the IspS promoter, preventing transcription. IPTG binds lad repressor and releases it from lacO sequences, allowing transcription. By using an inducible promoter system, isoprene synthesis may be controlled by the addition of an inducer. Nucleic acids encoding IspS or other isoprene pathway enzymes may also be combined with other nucleic acid sequences, polyadenylation signals, restriction enzyme sites, multiple cloning sites, other coding segments, and the like.

In certain embodiments, the strength and timing of expression of DXP pathway enzymes and an isoprene synthase (e.g., IspS) or mevalonate pathway enzymes and the isoprene synthase (e.g., IspS) may be modulated using methods known in the art to improve isoprene production. For example, varying promoter strength or gene copy number may be used to modulate expression levels. In another example, timing of expression may be modulated by using inducible promoter systems or polycistronic operons with arranged gene orders. For example, expression of DXP pathway enzymes and an isoprene synthase (e.g., IspS) or mevalonate pathway enzymes and the isoprene synthase (e.g., IspS) may be expressed during growth phase and stationary phase of culture or during stationary phase only. In another example, isoprene DXP pathway enzymes and IspS or mevalonate pathway enzymes and IspS may undergo ordered coexpression. Ordered co-expression of nucleic acids encoding various DXP pathway enzymes has been found to enhance isoprene production (Lv et al., 2012, Appl. Microbiol. Biotechnol., “Significantly enhanced production of isoprene by ordered coexpression of genes dxs, dxr, and idi in Escherichia coli,” published online Nov. 10, 2012).

### Codon Optimization

Expression of recombinant proteins is often difficult outside their original host. For example, variation in codon usage bias has been observed across different species of bacteria (Sharp et al., 2005, Nucl. Acids. Res. 33:1141-1153). Over-expression of recombinant proteins even within their native host may also be difficult. In certain embodiments of the invention, nucleic acids (e.g., a nucleic acid encoding isoprene synthase) that are to be introduced into microorganisms of the invention may undergo codon optimization to enhance protein expression. Codon optimization refers to alteration of codons in genes or coding regions of nucleic acids for transformation of an organism to reflect the typical codon usage of the host organism without altering the polypeptide for which the DNA encodes. In certain embodiments, an exogenous nucleic acid encoding IspS, other isoprene pathway components, or lycopene pathway components are codon optimized for expression in the host methanotrophic bacterium. Codon optimization methods for optimum gene expression in heterologous organisms are known in the art and have been previously described (see, e.g., Welch et al., 2009, PLoS One 4:e7002; Gustafsson et al., 2004, Trends Biotechnol. 22:346-353; Wu et al., 2007, Nucl. Acids Res. 35:D76-79; Villalobos et al., 2006, BMC Bioinformatics 7:285; U.S. Patent Publication 2011/0111413; and U.S. Patent Publication 2008/0292918).

Examples of isoprene synthase (e.g., IspS) polynucleotide sequences codon-optimized for expression in Methylococcus capsulatus Bath strain are provided in Table 3. SEQ ID NOs:15, 17, and 19 are truncated IspS sequences from Populus alba, Pueraria montana, and Salix, respectively, without their N-terminal plastid-targeting sequences. SEQ ID NOs:14, 16, and 18 are full length IspS sequences (with N-terminal plastid-targeting sequences) from Populus alba, Pueraria montana, and Salix, respectively.

TABLE 3
IspS polynucleotide sequences codon-optimized for expression in
Methylococcus capsulatus Bath strain

| | | SEQ ID |
|---|---|---|
| Species | Nucleotide sequence | NO. |
| Populus | ATGGCCACTGAACTTCTTTGTTTGCACCGCCCGATTTCCC | 14 |
| alba | TGACCCATAAGCTGTTTCGCAACCCTCTGCCCAAAGTTAT | |
| | CCAGGCAACCCCGCTGACGCTCAAGCTCCGGTGCAGCGTA | |
| | TCCACCGAAAATGTATCGTTCACCGAAACCGAAACTGAAG | |
| | CCCGTCGCAGCGCGAACTACGAGCCCAACTCGTGGGATTA | |
| | CGACTATCTGCTGAGCTCGGATACCGACGAATCCATCGAA | |
| | GTCTATAAGGACAAAGCCAAGAAGCTCGAAGCCGAGGTGC | |
| | GCCGTGAGATCAACAACGAGAAGGCCGAGTTCCTGACCCT | |
| | GTTGGAACTGATCGACAACGTCCAGCGCCTGGGCCTCGGC | |
| | TACCGGTTCGAGAGCGATATCCGGGGTGCCCTGGACCGTT | |
| | TCGTCAGCTCGGGCGGATTCGACGCAGTGACCAAAACGTC | |
| | GCTGCATGGGACGGCCCTGTCCTTCCGTCTGCTGCGCCAG | |
| | CATGGCTTCGAGGTGTCCCAGGAAGCCTTCAGCGGCTTCA | |
| | AGGATCAGAACGGAAACTTTCTGGAAAACTTGAAAGAGGA | |
| | CATCAAGGCCATCCTCAGCCTGTACGAGGCGTCCTTCCTG | |
| | GCCCTCGAAGGTGAAAACATCCTCGATGAAGCCAAGGTGT | |
| | TCGCAATCTCGCATCTTAAAGAGCTGTCCGAAGAGAAGAT | |
| | TGGCAAAGAGCTGGCCGAACAAGTCAACCACGCGTTGGAG | |
| | CTGCCGCTCCACCGGCGCACCCAGCGGCTGGAAGCGGTCT | |
| | GGTCGATCGAAGCCTACCGCAAGAAAGAGGACGCCAATCA | |
| | GGTCCTGCTGGAGCTCGCGATTCTGGATTACAATATGATC | |
| | CAGTCGGTCTATCAGCGCGATCTGCGCGAAACGTCCCGGT | |
| | GGTGGCGGCGTGTCGGCTTGGCGACCAAGTTGCACTTCGC | |
| | GCGTGACCGCTTGATCGAGAGCTTCTATTGGGCCGTCGGG | |
| | GTGGCCTTTGAGCCCCAGTACTCCGACTGCCGCAATAGCG | |
| | TGGCGAAGATGTTCAGCTTCGTTACCATCATCGACGACAT | |
| | CTACGACGTGTATGGCACGCTCGACGAGCTCGAACTGTTC | |
| | ACCGACGCCGTGGAACGTTGGGACGTCAACGCCATCAATG | |
| | ATCTCCCCGACTACATGAAGCTGTGCTTCCTGGCGTTGTA | |
| | TAACACCATCAACGAGATTGCCTACGATAACCTCAAGGAC | |
| | AAGGGCGAGAACATCCTGCCGTACTTGACCAAGGCCTGGG | |
| | CCGATTTGTGCAACGCCTTTCTGCAGGAAGCAAAGTGGCT | |
| | GTACAACAAATCCACGCCGACGTTCGACGACTATTTCGGC | |
| | AATGCATGGAAATCGAGCTCGGGTCCTCTGCAACTTGTGT | |
| | TCGCGTACTTCGCCGTCGTGCAGAATATCAAGAAAGAAGA | |
| | AATCGAGAACCTTCAGAAATATCATGACACCATCAGCCGT | |
| | CCATCGCACATCTTTCGCCTGTGCAACGACCTCGCGTCCG | |
| | CATCCGCCGAGATCGCACGCGGCGAAACGGCCAATTCGGT | |
| | GTCCTGCTACATGCGGACCAAGGGCATCTCGGAAGAGCTG | |
| | GCGACGGAATCCGTGATGAACCTGATCGATGAAACCTGGA | |
| | AGAAGATGAACAAAGAGAAGCTCGGCGGGAGCCTGTTCGC | |
| | GAAGCCCTTCGTCGAAACCGCAATTAACCTGGCACGCCAA | |
| | TCCCACTGTACCTACCATAACGGAGATGCCCACACGAGCC | |
| | CGGACGAGCTGACTCGCAAGCGCGTCCTTTCGGTCATCAC | |
| | CGAGCCGATCCTGCCGTTCGAGCGGTAA | |
| Populus | ATGTGCAGCGTATCCACCGAAAATGTATCGTTCACCGAAA | 15 |
| alba | CCGAAACTGAAGCCCGTCGCAGCGCGAACTACGAGCCCAA | |
| (truncated) | CTCGTGGGATTACGACTATCTGCTGAGCTCGGATACCGAC | |
| | GAATCCATCGAAGTCTATAAGGACAAAGCCAAGAAGCTCG | |
| | AAGCCGAGGTGCGCCGTGAGATCAACAACGAGAAGGCCGA | |
| | GTTCCTGACCCTGTTGGAACTGATCGACAACGTCCAGCGC | |
| | CTGGGCCTCGGCTACCGGTTCGAGAGCGATATCCGGGGTG | |
| | CCCTGGACCGTTTCGTCAGCTCGGGCGGATTCGACGCAGT | |
| | GACCAAAACGTCGCTGCATGGGACGGCCCTGTCCTTCCGT | |
| | CTGCTGCGCCAGCATGGCTTCGAGGTGTCCCAGGAAGCCT | |
| | TCAGCGGCTTCAAGGATCAGAACGGAAACTTTCTGGAAAA | |
| | CTTGAAAGAGGACATCAAGGCCATCCTCAGCCTGTACGAG | |
| | GCGTCCTTCCTGGCCCTCGAAGGTGAAAACATCCTCGATG | |
| | AAGCCAAGGTGTTCGCAATCTCGCATCTTAAAGAGCTGTC | |
| | CGAAGAGAAGATTGGCAAAGAGCTGGCCGAACAAGTCAAC | |
| | CACGCGTTGGAGCTGCCGCTCCACCGGCGCACCCAGCGGC | |
| | TGGAAGCGGTCTGGTCGATCGAAGCCTACCGCAAGAAAGA | |
| | GGACGCCAATCAGGTCCTGCTGGAGCTCGCGATTCTGGAT | |
| | TACAATATGATCCAGTCGGTCTATCAGCGCGATCTGCGCG | |
| | AAACGTCCCGGTGGTGGCGGCGTGTCGGCTTGGCGACCAA | |
| | GTTGCACTTCGCGCGTGACCGCTTGATCGAGAGCTTCTAT | |
| | TGGGCCGTCGGGGTGGCCTTTGAGCCCCAGTACTCCGACT | |
| | GCCGCAATAGCGTGGCGAAGATGTTCAGCTTCGTTACCAT | |
| | CATCGACGACATCTACGACGTGTATGGCACGCTCGACGAG | |
| | CTCGAACTGTTCACCGACGCCGTGGAACGTTGGGACGTCA | |
| | ACGCCATCAATGATCTCCCCGACTACATGAAGCTGTGCTT | |
| | CCTGGCGTTGTATAACACCATCAACGAGATTGCCTACGAT | |
| | AACCTCAAGGACAAGGGCGAGAACATCCTGCCGTACTTGA | |
| | CCAAGGCCTGGGCCGATTTGTGCAACGCCTTTCTGCAGGA | |
| | AGCAAAGTGGCTGTACAACAAATCCACGCCGACGTTCGAC | |
| | GACTATTTCGGCAATGCATGGAAATCGAGCTCGGGTCCTC | |
| | TGCAACTTGTGTTCGCGTACTTCGCCGTCGTGCAGAATAT | |
| | CAAGAAAGAAGAAATCGAGAACCTTCAGAAATATCATGAC | |
| | ACCATCAGCCGTCCATCGCACATCTTTCGCCTGTGCAACG | |
| | ACCTCGCGTCCGCATCCGCCGAGATCGCACGCGGCGAAAC | |
| | GGCCAATTCGGTGTCCTGCTACATGCGGACCAAGGGCATC | |
| | TCGGAAGAGCTGGCGACGGAATCCGTGATGAACCTGATCG | |
| | ATGAAACCTGGAAGAAGATGAACAAAGAGAAGCTCGGCGG | |
| | GAGCCTGTTCGCGAAGCCCTTCGTCGAAACCGCAATTAAC | |
| | CTGGCACGCCAATCCCACTGTACCTACCATAACGGAGATG | |
| | CCCACACGAGCCCGGACGAGCTGACTCGCAAGCGCGTCCT | |
| | TTCGGTCATCACCGAGCCGATCCTGCCGTTCGAGCGGTAA | |
| Pueraria | ATGGCCACCAATCTGCTCTGCCTGTCGAATAAACTGTCCA | 16 |
| montana | GCCCCACGCCCACGCCGTCCACGCGGTTCCCGCAGTCCAA | |
| | GAACTTCATTACCCAGAAAACCAGCCTCGCCAACCCGAAG | |
| | CCATGGCGCGTGATCTGCGCAACCTCGTCCCAATTCACCC | |
| | AGATCACGGAACACAACTCGCGTCGCTCGGCCAACTACCA | |
| | GCCTAATTTGTGGAACTTCGAGTTCCTGCAGAGCTTGGAG | |
| | AACGATCTGAAGGTCGAGAAGCTGGAAGAGAAAGCCACCA | |
| | AGCTCGAAGAAGAGGTCCGTTGCATGATCAACCGCGTCGA | |
| | CACTCAGCCGCTCTCCCTGCTGGAGCTTATCGACGACGTC | |
| | CAGCGCCTCGGCTTGACTTACAAGTTCGAGAAAGACATTA | |
| | TCAAGGCCCTTGAGAATATCGTCCTGCTGGATGAAAACAA | |
| | AAAGAACAAGTCGGATCTGCATGCGACCGCCCTGAGCTTC | |
| | CGGCTGCTGCGCCAGCACGGCTTTGAGGTCAGCCAAGACG | |
| | TATTCGAACGCTTCAAGGATAAAGAAGGCGGGTTTTCCGG | |
| | CGAATTGAAAGGCGACGTGCAGGGCTTGCTCTCGCTGTAC | |
| | GAGGCCAGCTACCTGGGCTTTGAGGGTGAAAATCTGCTCG | |
| | AAGAGGCGCGTACCTTCAGCATCACGCATCTGAAGAATAA | |
| | CCTCAAAGAGGGCATCAACACCAAGGTGGCCGAACAAGTG | |
| | TCCCACGCGCTGGAACTGCCATACCATCAACGGCTGCATC | |
| | GCCTGGAAGCGCGCTGGTTCTTGGACAAGTATGAACCCAA | |
| | AGAACCTCACCATCAGCTGCTTCTGGAGCTCGCCAAGTTG | |
| | GACTTCAACATGGTCCAGACCTTGCACCAGAAAGAACTGC | |
| | AGGACTTGTCCCGGTGGTGGACCGAAATGGGACTGGCGTC | |
| | CAAGCTTGACTTCGTCCGCGATCGCCTCATGGAAGTGTAC | |
| | TTTTGGGCCCTCGGAATGGCACCGGACCCGCAGTTCGGCG | |
| | AGTGCCGCAAAGCAGTTACCAAGATGTTCGGCCTGGTCAC | |
| | CATTATCGACGATGTCTACGACGTATACGGGACGTTGGAT | |
| | GAGCTGCAACTGTTCACGGACGCCGTGGAGCGGTGGGACG | |
| | TCAACGCCATCAACACGCTCCCCGACTATATGAAGCTCTG | |
| | CTTCCTGGCATTGTACAATACCGTGAACGACACCTCGTAT | |
| | TCCATTCTGAAAGAAAAAGGACACAATAACCTGTCCTATC | |
| | TGACCAAGTCCTGGCGTGAGCTGTGCAAGGCGTTCCTGCA | |
| | AGAAGCCAAGTGGAGCAATAACAAGATCATCCCCGCGTTC | |
| | TCGAAGTATCTTGAGAACGCATCCGTGTCGAGCAGCGGGG | |
| | TCGCCCTGCTGGCCCCGTCGTACTTCAGCGTATGTCAGCA | |
| | GCAGGAAGATATCTCGGACCACGCGCTGCGTAGCCTTACG | |
| | GACTTCCATGGCCTCGTCCGGTCGAGCTGCGTGATCTTCC | |
| | GTTTGTGCAACGACCTGGCGACCTCGGCCGCAGAACTGGA | |
| | GCGGGGTGAAACCACCAACAGCATCATCTCGTACATGCAC | |
| | GAGAACGATGGCACGTCGGAAGAGCAGGCACGCGAAGAGC | |
| | TGCGTAAGCTGATCGACGCCGAGTGGAAGAAAATGAACCG | |
| | CGAACGCGTCAGCGACTCCACCCTGCTGCCGAAGGCCTTC | |
| | ATGGAAATCGCCGTGAACATGGCACGTGTGTCCCATTGTA | |
| | CTTATCAGTACGGCGATGGCCTGGGTCGCCCCGACTATGC | |
| | CACGGAGAACCGGATCAAGCTCCTGTTGATCGATCCGTTC | |
| | CCGATCAACCAGCTGATGTACGTGTAA | |
| Pueraria | ATGTGCGCAACCTCGTCCCAATTCACCCAGATCACGGAAC | 17 |
| montana | ACAACTCGCGTCGCTCGGCCAACTACCAGCCTAATTTGTG | |
| (truncated) | GAACTTCGAGTTCCTGCAGAGCTTGGAGAACGATCTGAAG | |
| | GTCGAGAAGCTGGAAGAGAAAGCCACCAAGCTCGAAGAAG | |
| | AGGTCCGTTGCATGATCAACCGCGTCGACACTCAGCCGCT | |
| | CTCCCTGCTGGAGCTTATCGACGACGTCCAGCGCCTCGGC | |
| | TTGACTTACAAGTTCGAGAAAGACATTATCAAGGCCCTTG | |
| | AGAATATCGTCCTGCTGGATGAAAACAAAAAGAACAAGTC | |
| | GGATCTGCATGCGACCGCCCTGAGCTTCCGGCTGCTGCGC | |
| | CAGCACGGCTTTGAGGTCAGCCAAGACGTATTCGAACGCT | |
| | TCAAGGATAAAGAAGGCGGGTTTTCCGGCGAATTGAAAGG | |
| | CGACGTGCAGGGCTTGCTCTCGCTGTACGAGGCCAGCTAC | |
| | CTGGGCTTTGAGGGTGAAAATCTGCTCGAAGAGGCGCGTA | |
| | CCTTCAGCATCACGCATCTGAAGAATAACCTCAAAGAGGG | |
| | CATCAACACCAAGGTGGCCGAACAAGTGTCCCACGCGCTG | |
| | GAACTGCCATACCATCAACGGCTGCATCGCCTGGAAGCGC | |
| | GCTGGTTCTTGGACAAGTATGAACCCAAAGAACCTCACCA | |
| | TCAGCTGCTTCTGGAGCTCGCCAAGTTGGACTTCAACATG | |
| | GTCCAGACCTTGCACCAGAAAGAACTGCAGGACTTGTCCC | |
| | GGTGGTGGACCGAAATGGGACTGGCGTCCAAGCTTGACTT | |
| | CGTCCGCGATCGCCTCATGGAAGTGTACTTTTGGGCCCTC | |
| | GGAATGGCACCGGACCCGCAGTTCGGCGAGTGCCGCAAAG | |
| | CAGTTACCAAGATGTTCGGCCTGGTCACCATTATCGACGA | |
| | TGTCTACGACGTATACGGGACGTTGGATGAGCTGCAACTG | |
| | TTCACGGACGCCGTGGAGCGGTGGGACGTCAACGCCATCA | |
| | ACACGCTCCCCGACTATATGAAGCTCTGCTTCCTGGCATT | |
| | GTACAATACCGTGAACGACACCTCGTATTCCATTCTGAAA | |
| | GAAAAAGGACACAATAACCTGTCCTATCTGACCAAGTCCT | |
| | GGCGTGAGCTGTGCAAGGCGTTCCTGCAAGAAGCCAAGTG | |
| | GAGCAATAACAAGATCATCCCCGCGTTCTCGAAGTATCTT | |
| | GAGAACGCATCCGTGTCGAGCAGCGGGGTCGCCCTGCTGG | |
| | CCCCGTCGTACTTCAGCGTATGTCAGCAGCAGGAAGATAT | |
| | CTCGGACCACGCGCTGCGTAGCCTTACGGACTTCCATGGC | |
| | CTCGTCCGGTCGAGCTGCGTGATCTTCCGTTTGTGCAACG | |
| | ACCTGGCGACCTCGGCCGCAGAACTGGAGCGGGGTGAAAC | |
| | CACCAACAGCATCATCTCGTACATGCACGAGAACGATGGC | |
| | ACGTCGGAAGAGCAGGCACGCGAAGAGCTGCGTAAGCTGA | |
| | TCGACGCCGAGTGGAAGAAAATGAACCGCGAACGCGTCAG | |
| | CGACTCCACCCTGCTGCCGAAGGCCTTCATGGAAATCGCC | |
| | GTGAACATGGCACGTGTGTCCCATTGTACTTATCAGTACG | |
| | GCGATGGCCTGGGTCGCCCCGACTATGCCACGGAGAACCG | |
| | GATCAAGCTCCTGTTGATCGATCCGTTCCCGATCAACCAG | |
| | CTGATGTACGTGTAA | |
| Salix | ATGGCCACTGAACTTCTGTGCTTGCACCGTCCCATTTCGC | 18 |
| | TCACCCCTAAACTGTTCCGCAACCCGCTCCCGAAGGTAAT | |
| | CCTGGCGACGCCGCTGACCCTGAAGCTGCGGTGCAGCGTA | |
| | TCCACCGAAAACGTGAGCTTTACTGAAACCGAAACCGAAA | |
| | CGCGTCGCTCGGCGAACTACGAACCCAATTCCTGGGATTA | |
| | TGACTACCTTCTGTCGTCCGACACGGACGAGTCGATCGAG | |
| | GTGTATAAGGATAAGGCCAAGAAGCTTGAGGCGGAAGTCC | |
| | GTCGGGAGATCAACAACGAGAAGGCGGAGTTCCTGACGCT | |
| | GCTCGAACTGATTGACAACGTCCAGCGCCTCGGCCTGGGC | |
| | TATCGCTTCGAGTCCGATATCCGTCGCGCACTCGACCGCT | |
| | TCGTTTCGTCCGGTGGCTTCGACGCAGTGACGAAAACCTC | |
| | GCTGCATGCCACCGCGCTGTCGTTCCGCTTCCTGCGCCAG | |
| | CACGGATTCGAGGTCAGCCAGGAAGCGTTCGGCGGGTTCA | |
| | AGGACCAGAACGGGAATTTCCTGGAAAATCTGAAAGAAGA | |
| | TATCAAAGCCATCTTGTCGCTGTACGAGGCGTCGTTTCTC | |
| | GCGCTCGAAGGCGAGAACATTCTCGACGAAGCGAAGGTGT | |
| | TCGCCATCTCGCACCTGAAAGAGCTCTCCGAAGAGAAGAT | |
| | CGGCAAAGACTTGGCCGAGCAAGTCAATCACGCCCTGGAG | |
| | TTGCCCCTGCATCGCCGCACCCAGCGCTTGGAAGCCGTTT | |
| | GGAGCATTGAAGCCTATCGTAAGAAAGAGGACGCCAACCA | |
| | AGTCCTGCTGGAGCTGGCCATCCTGGACTACAACATGATC | |
| | CAGTCCGTGTACCAGCGGGACTTGCGCGAAACCAGCCGGT | |
| | GGTGGCGTCGCGTCGGCCTCGCCACCAAGCTGCACTTCGC | |
| | ACGCGACCGCCTGATCGAGTCCTTCTACTGGGCCGTGGGC | |
| | GTCGCATTCGAGCCGCAATATAGCGACTGCCGGAACAGCG | |
| | TGGCAAAGATGTTCAGCTTCGTGACCATCATCGACGATAT | |
| | CTATGACGTGTATGGGACGCTTGACGAACTGGAGCTGTTT | |
| | ACGGATGCCGTCGAGCGGTGGGACGTCAATGCCATCAACG | |
| | ATTTGCCGGACTACATGAAGCTGTGCTTCCTGGCCTTGTA | |
| | TAACACTATCAACGAGATCGCCTACGATAACCTGAAAGAA | |
| | AAGGGTGAGAACATCCTGCCCTACCTCACCAAGGCCTGGG | |
| | CCGACCTGTGTAACGCCTTTCTGCAGGAAGCCAAGTGGCT | |
| | CTACAACAAGTCCACCCCAACCTTCGACGATTACTTCGGA | |
| | AATGCCTGGAAGAGCAGCTCCGGACCTCTCCAGCTGGTGT | |
| | TCGCATACTTCGCCGTCGTGCAGAACATCAAGAAAGAAGA | |
| | GATCGAAAACTTGCAGAAGTACCACGATATCATCAGCCGT | |
| | CCCTCGCACATCTTCCGGCTCTGCAACGACCTTGCAAGCG | |
| | CGTCCGCGGAGATCGCACGGGGCGAAACGGCCAACTCGGT | |
| | GAGCTGCTACATGCGCACCAAGGGCATCTCGGAAGAACTT | |
| | GCGACGGAGTCCGTCATGAACTTGATCGACGAAACCTGGA | |
| | AGAAAATGAATAAAGAGAAACTCGGCGGCAGCCTGTTCCC | |
| | GAAGCCATTCGTCGAAACCGCCATCAACCTGGCGCGTCAG | |
| | TCGCATTGCACCTACCATAATGGCGATGCCCATACGTCGC | |
| | CGGATGAACTGACCCGTAAGCGGGTCCTGTCCGTCATCAC | |
| | CGAGCCGATTCTGCCGTTCGAGCGCTAA | |
| Salix | ATGTGCAGCGTATCCACCGAAAACGTGAGCTTTACTGAAA | 19 |
| (truncated) | CCGAAACCGAAACGCGTCGCTCGGCGAACTACGAACCCAA | |
| | TTCCTGGGATTATGACTACCTTCTGTCGTCCGACACGGAC | |
| | GAGTCGATCGAGGTGTATAAGGATAAGGCCAAGAAGCTTG | |
| | AGGCGGAAGTCCGTCGGGAGATCAACAACGAGAAGGCGGA | |
| | GTTCCTGACGCTGCTCGAACTGATTGACAACGTCCAGCGC | |
| | CTCGGCCTGGGCTATCGCTTCGAGTCCGATATCCGTCGCG | |
| | CACTCGACCGCTTCGTTTCGTCCGGTGGCTTCGACGCAGT | |
| | GACGAAAACCTCGCTGCATGCCACCGCGCTGTCGTTCCGC | |
| | TTCCTGCGCCAGCACGGATTCGAGGTCAGCCAGGAAGCGT | |
| | TCGGCGGGTTCAAGGACCAGAACGGGAATTTCCTGGAAAA | |
| | TCTGAAAGAAGATATCAAAGCCATCTTGTCGCTGTACGAG | |
| | GCGTCGTTTCTCGCGCTCGAAGGCGAGAACATTCTCGACG | |
| | AAGCGAAGGTGTTCGCCATCTCGCACCTGAAAGAGCTCTC | |
| | CGAAGAGAAGATCGGCAAAGACTTGGCCGAGCAAGTCAAT | |
| | CACGCCCTGGAGTTGCCCCTGCATCGCCGCACCCAGCGCT | |
| | TGGAAGCCGTTTGGAGCATTGAAGCCTATCGTAAGAAAGA | |
| | GGACGCCAACCAAGTCCTGCTGGAGCTGGCCATCCTGGAC | |
| | TACAACATGATCCAGTCCGTGTACCAGCGGGACTTGCGCG | |
| | AAACCAGCCGGTGGTGGCGTCGCGTCGGCCTCGCCACCAA | |
| | GCTGCACTTCGCACGCGACCGCCTGATCGAGTCCTTCTAC | |
| | TGGGCCGTGGGCGTCGCATTCGAGCCGCAATATAGCGACT | |
| | GCCGGAACAGCGTGGCAAAGATGTTCAGCTTCGTGACCAT | |
| | CATCGACGATATCTATGACGTGTATGGGACGCTTGACGAA | |
| | CTGGAGCTGTTTACGGATGCCGTCGAGCGGTGGGACGTCA | |
| | ATGCCATCAACGATTTGCCGGACTACATGAAGCTGTGCTT | |
| | CCTGGCCTTGTATAACACTATCAACGAGATCGCCTACGAT | |
| | AACCTGAAAGAAAAGGGTGAGAACATCCTGCCCTACCTCA | |
| | CCAAGGCCTGGGCCGACCTGTGTAACGCCTTTCTGCAGGA | |
| | AGCCAAGTGGCTCTACAACAAGTCCACCCCAACCTTCGAC | |
| | GATTACTTCGGAAATGCCTGGAAGAGCAGCTCCGGACCTC | |
| | TCCAGCTGGTGTTCGCATACTTCGCCGTCGTGCAGAACAT | |
| | CAAGAAAGAAGAGATCGAAAACTTGCAGAAGTACCACGAT | |
| | ATCATCAGCCGTCCCTCGCACATCTTCCGGCTCTGCAACG | |
| | ACCTTGCAAGCGCGTCCGCGGAGATCGCACGGGGCGAAAC | |
| | GGCCAACTCGGTGAGCTGCTACATGCGCACCAAGGGCATC | |
| | TCGGAAGAACTTGCGACGGAGTCCGTCATGAACTTGATCG | |
| | ACGAAACCTGGAAGAAAATGAATAAAGAGAAACTCGGCGG | |
| | CAGCCTGTTCCCGAAGCCATTCGTCGAAACCGCCATCAAC | |
| | CTGGCGCGTCAGTCGCATTGCACCTACCATAATGGCGATG | |
| | CCCATACGTCGCCGGATGAACTGACCCGTAAGCGGGTCCT | |
| | GTCCGTCATCACCGAGCCGATTCTGCCGTTCGAGCGCTAA | |

### Exemplary Culture of Methanotrophs

Non-naturally occurring methanotrophic bacteria as described herein may be cultured using a materials and methods well known in the art. In certain embodiments, non-naturally occurring methanotrophic bacteria are cultured under conditions permitting expression of one or more nucleic acids (e.g., IspS) introduced into the host methanotrophic cells.

A classical batch culturing method is a closed system where the composition of the media is set at the beginning of the culture and not subject to external alterations during the culture process. Thus, at the beginning of the culturing process, the media is inoculated with the desired organism or organism and growth or metabolic activity is permitted to occur without adding anything to the system. Typically, however, a “batch” culture is batch with respect to the addition of carbon source and attempts are often made at controlling factors such as pH and oxygen concentration. In batch systems, the metabolite and biomass compositions of the system change constantly up to the time the culture is terminated. Within batch cultures, cells moderate through a static lag phase to a high growth logarithmic phase and finally to a stationary phase where growth rate is diminished or halted. If untreated, cells in the stationary phase will eventually die. Cells in log phase are often responsible for the bulk production of end product or intermediate in some systems. Stationary or post-exponential phase production can be obtained in other systems.

The Fed-Batch system is a variation on the standard batch system. Fed-Batch culture processes comprise a typical batch system with the modification that the substrate is added in increments as the culture progresses. Fed-Batch systems are useful when catabolite repression is apt to inhibit the metabolism of the cells and where it is desirable to have limited amounts of substrate in the media. Measurement of the actual substrate concentration in Fed-Batch systems is difficult and is therefore estimated on the basis of the changes of measureable factors, such as pH, dissolved oxygen, and the partial pressure of waste gases such as CO2. Batch and Fed-Batch culturing methods are common and known in the art (see, e.g., Thomas D. Brock, Biotechnology: A Textbook of Industrial Microbiology, 2^{nd} Ed. (1989) Sinauer Associates, Inc., Sunderland, Mass.; Deshpande, 1992, Appl. Biochem. Biotechnol. 36:227, incorporated by reference in its entirety).

Continuous cultures are “open” systems where a defined culture media is added continuously to a bioreactor and an equal amount of conditioned media is removed simultaneously for processing. Continuous cultures generally maintain the cells at a constant high liquid phase density where cells are primarily in logarithmic phase growth. Alternatively, continuous culture may be practiced with immobilized cells where carbon and nutrients are continuously added and valuable products, by-products, and waste products are continuously removed from the cell mass. Cell immobilization may be performed using a wide range of solid supports composed of natural or synthetic materials.

Continuous or semi-continuous culture allows for the modulation of one factor or any number of factors that affect cell growth or end product concentration. For example, one method will maintain a limited nutrient, such as the carbon source or nitrogen level, at a fixed rate and allow all other parameters to modulate. In other systems, a number of factors affecting growth can be altered continuously while the cell concentration, measured by media turbidity, is kept constant. Continuous systems strive to maintain steady state growth conditions and thus the cell loss due to media being drawn off must be balanced against the cell growth rate in the culture. Methods of modulating nutrients and growth factors for continuous culture processes, as well as techniques for maximizing the rate of product formation, are well known in the art, and a variety of methods are detailed by Brock, supra.

Methanotrophic bacteria may also be immobilized on a solid substrate as whole cell catalysts and subjected to fermentation conditions for isoprene production.

Methanotrophic bacteria provided in the present disclosure may be grown as an isolated pure culture, with a heterologous non-methanotrophic organism(s) that may aid with growth, or one or more different strains/or species of methanotrophic bacteria may be combined to generate a mixed culture.

Any carbon source, carbon containing compounds capable of being metabolized by methanotrophic bacteria, also referred to as carbon feedstock, may be used to cultivate non-naturally occurring methanotrophic bacteria described herein. A carbon feedstock may be used for maintaining viability, growing methanotrophic bacteria, or converted into isoprene.

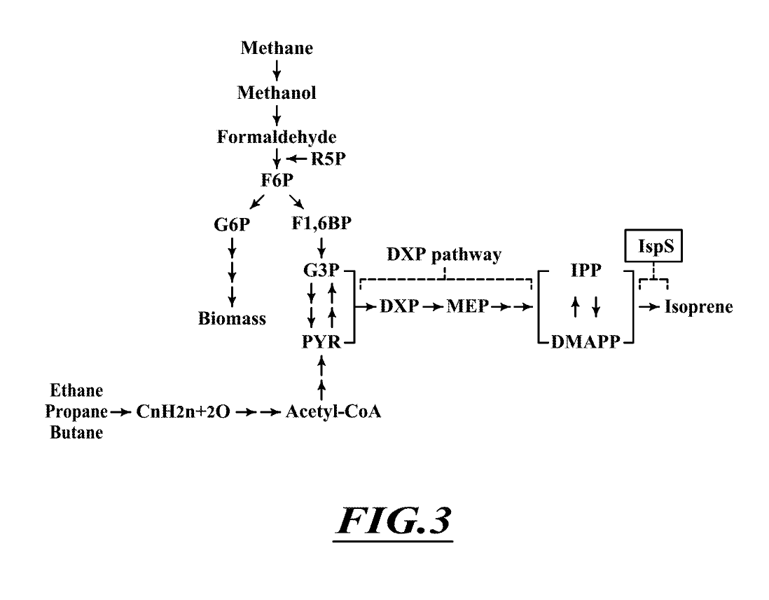

In certain embodiments, non-naturally occurring methanotrophic bacteria genetically engineered with one or more isoprene pathway enzymes as described herein, is capable of converting a carbon feedstock into isoprene, wherein the carbon feedstock is a C1 substrate. A C1 substrate includes, but is not limited to, methane, methanol, natural gas, and unconventional natural gas. Non-naturally occurring methanotrophic bacteria may also convert non-C1 substrates, such as multi-carbon substrates, into isoprene. Non-naturally occurring methanotrophic bacteria may endogenously have the ability to convert multi-carbon substrates such as light alkanes (ethane, propane, and butane), into isoprene once isoprene biosynthetic capability has been introduced into the bacteria (see FIG. 3). Alternatively, non-naturally occurring methanotrophic bacteria may require additional genetic engineering to use alternative carbon feedstocks (see, e.g., U.S. Provisional Application 61/718,024 filed Oct. 24, 2012, “Engineering of Multi-Carbon Substrate Utilization Pathways in Methanotrophic Bacteria”, incorporated by reference in its entirety), which can then be converted into isoprene according to the present disclosure. Methanotrophic bacteria may be provided a pure or relatively pure carbon feedstock comprising mostly of a single carbon substrate, such as methane or dry natural gas. Methanotrophic bacteria may also be provided a mixed carbon feedstock, such as wet natural gas, which includes methane and light alkanes.

### Construction of Non-Naturally Occurring Methanotrophic Bacteria

Recombinant DNA and molecular cloning techniques used herein are well known in the art are described in Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999).

Recombinant methods for introduction of heterologous nucleic acids in methanotrophic bacteria are known in the art. Expression systems and expression vectors useful for the expression of heterologous nucleic acids in methanotrophic bacteria are known. Vectors or cassettes useful for the transformation of methanotrophic bacteria are known.

Electroporation of C1 metabolizing bacteria has been previously described in Toyama et al., 1998, FEMS Microbiol. Lett. 166:1-7 (Methylobacterium extorquens); Kim and Wood, 1997, Appl. Microbiol. Biotechnol. 48:105-108 (Methylophilus methylotrophus AS1); Yoshida et al., 2001, Biotechnol. Lett. 23:787-791 (Methylobacillus sp. strain 12S), and US2008/0026005 (Methylobacterium extorquens).

Bacterial conjugation, which refers to a particular type of transformation involving direct contact of donor and recipient cells, is more frequently used for the transfer of nucleic acids into methanotrophic bacteria. Bacterial conjugation involves mixing “donor” and “recipient” cells together in close contact with each other. Conjugation occurs by formation of cytoplasmic connections between donor and recipient bacteria, with unidirectional transfer of newly synthesized donor nucleic acids into the recipient cells. A recipient in a conjugation reaction is any cell that can accept nucleic acids through horizontal transfer from a donor bacterium. A donor in a conjugation reaction is a bacterium that contains a conjugative plasmid, conjugative transposon, or mobilized plasmid. The physical transfer of the donor plasmid can occur through a self-transmissible plasmid or with the assistance of a “helper” plasmid. Conjugations involving C1 metabolizing bacteria, including methanotrophic bacteria, have been previously described in Stolyar et al., 1995, Mikrobiologiya 64:686-691; Martin and Murrell, 1995, FEMS Microbiol. Lett. 127:243-248; Motoyama et al., 1994, Appl. Micro. Biotech. 42:67-72; Lloyd et al., 1999, Archives of Microbiology 171:364-370; and Odom et al., PCT Publication WO 02/18617; Ali et al., 2006, Microbiol. 152:2931-2942.

As described herein, it may be desirable to overexpress various upstream isoprene pathway genes to enhance production. Overexpression of endogenous or heterologous nucleic acids may be achieved using methods known in the art, such as multi-copy plasmids or strong promoters. Use of multi-copy expression systems in methanotrophs is known in the art (see, e.g., Cardy and Murrell, 1990 J. Gen. Microbiol. 136:343-352; Sharpe et al., 2007, Appl. Environ. Microbiol. 73:1721-1728). For example, a transposon-based multicopy expression of heterologous genes in Methylobacterium has been described (see, e.g. U.S. Patent Publication 2008/0026005). Suitable homologous or heterologous promoters for high expression of exogenous nucleic acids may also be utilized. For example, U.S. Pat. No. 7,098,005 describes the use of promoters that are highly expressed in the presence of methane or methanol for heterologous gene expression in methanotrophic bacteria. Additional promoters that may be used include deoxy-xylulose phosphate synthase methanol dehydrogenase operon promoter (Springer et al., 1998, FEMS Microbiol. Lett. 160:119-124); the promoter for PHA synthesis (Foellner et al. 1993, Appl. Microbiol. Biotechnol. 40:284-291); or promoters identified from native plasmid in methylotrophs (EP296484). Non-native promoters that may be used include the lac operon Plac promoter (Toyama et al., 1997, Microbiology 143:595-602) or a hybrid promoter such as Ptrc (Brosius et al., 1984, Gene 27:161-172). Additional promoters that may be used include leaky promoters or inducible promoter systems. For example, a repressor/operator system of recombinant protein expression in methylotrophic and methanotrophic bacteria has been described in U.S. Pat. No. 8,216,821.

Alternatively, disruption of certain genes may be desirable to eliminate competing energy or carbon sinks, enhance accumulation of isoprene pathway precursors, or prevent further metabolism of isoprene. Selection of genes for disruption may be determined based on empirical evidence. Candidate genes for disruption may include IspA. Methanotrophic bacteria are known to possess carotenoid biosynthetic pathways that may compete for isoprene precursors DMAPP and IPP (see, U.S. Pat. No. 6,969,595). IspA refers to a geranyltransferase or farnesyl diphosphate synthase enzyme that catalyzes a sequence of three prenyltransferase reactions in which geranyl diphosphate (GPP), farnesyl diphosphate (FPP), and geranylgeranyl diphosphate (GGPP) are formed from DMAPP and IPP. Various methods for down-regulating, inactivating, knocking-out, or deleting endogenous gene function in methanotrophic bacteria are known in the art. For example, targeted gene disruption is an effective method for gene down-regulation where a foreign DNA is inserted into a structural gene to disrupt transcription. Genetic cassettes comprising the foreign insertion DNA (usually a genetic marker) flanked by sequence having a high degree of homology to a portion of the target host gene to be disrupted are introduced into host methanotrophic bacteria. Foreign DNA disrupts the target host gene via native DNA replication mechanisms. Allelic exchange to construct deletion/insertional mutants in C₁ metabolizing bacteria, including methanotrophic bacteria, have been described in Toyama and Lidstrom, 1998, Microbiol. 144:183-191; Stolyar et al., 1999, Microbiol. 145:1235-1244; Ali et al., 2006, Microbiology 152:2931-2942; Van Dien et al., 2003, Microbiol. 149:601-609; Martin and Murrell, 2006, FEMS Microbiol. Lett. 127:243-248.

Nucleic acids that are transformed into host methanotrophic bacteria, such as nucleic acids encoding IspS, DXP pathway enzymes, mevalonate pathway enzymes, or lycopene pathway enzymes, may be introduced as separate nucleic acid molecules, on a polycistronic nucleic acid molecule, on a single nucleic acid molecule encoding a fusion protein, or a combination thereof. If more than one nucleic acid molecule is introduced into host methanotrophic bacteria, they may be introduced in various orders, including random order or sequential order according to the relevant metabolic pathway. In certain embodiments, when multiple nucleic acids encoding multiple enzymes from a selected biosynthetic pathway are transformed into host methanotrophic bacteria, they are transformed in a way to retain sequential order consistent with that of the selected biosynthetic pathway.

### Methods of Producing Isoprene

Methods are provided herein for producing isoprene, comprising: culturing a non-naturally occurring methanotrophic bacterium comprising an exogenous nucleic acid encoding isoprene synthase in the presence of a carbon feedstock under conditions sufficient to produce isoprene. Methods for growth and maintenance of methanotrophic bacterial cultures are well known in the art. Various embodiments of non-naturally occurring methanotrophic bacteria described herein may be used in the methods of producing isoprene.

In certain embodiments, isoprene is produced during a specific phase of cell growth (e.g., lag phase, log phase, stationary phase, or death phase). It may be desirable for carbon from feedstock to be converted to isoprene rather than to growth and maintenance of methanotrophic bacteria. In some embodiments, non-naturally occurring methanotrophic bacteria as provided herein are cultured to a low to medium cell density (OD₆₀₀) and then production of isoprene is initiated. In some embodiments, isoprene is produced while methanotrophic bacteria are no longer dividing or dividing very slowly. In some embodiments, isoprene is produced only during stationary phase. In some embodiments, isoprene is produced during log phase and stationary phase.

The fermenter off-gas comprising isoprene produced by non-naturally occurring methanotrophic bacteria provided herein may further comprise other organic compounds associated with biological fermentation processes. For example, biological by-products of fermentation may include one or more of the following: alcohols, epoxides, aldehydes, ketones, and esters. In certain embodiments, the fermenter off-gas may contain one or more of the following alcohols: methanol, ethanol, butanol, or propanol. In certain embodiments, the fermenter off-gas may contain one or more of the following epoxides: ethylene oxide, propylene oxide, or butene oxide. Other compounds, such as H₂O, CO, CO₂, CO N₂, H₂, O₂, and un-utilized carbon feedstocks, such as methane, ethane, propane, and butane, may also be present in the fermenter off-gas.

In certain embodiments, non-naturally occurring methanotrophic bacteria provided herein produce isoprene at about 0.001 g/L of culture to about 500 g/L of culture. In some embodiments, the amount of isoprene produced is about 1 g/L of culture to about 100 g/L of culture. In some embodiments, the amount of isoprene produced is about 0.001 g/L, 0.01 g/L, 0.025 g/L, 0.05 g/L, 0.1 g/L, 0.15 g/L, 0.2 g/L, 0.25 g/L, 0.3 g/L, 0.4 g/L, 0.5 g/L, 0.6 g/L, 0.7 g/L, 0.8 g/L, 0.9 g/L, 1 g/L, 2.5 g/L, 5 g/L, 7.5 g/L, 10 g/L, 12.5 g/L, 15 g/L, 20 g/L, 25 g/L, 30 g/L, 35 g/L, 40 g/L, 45 g/L, 50 g/L, 60 g/L, 70 g/L, 80 g/L, 90 g/L, 100 g/L, 125 g/L, 150 g/L, 175 g/L, 200 g/L, 225 g/L, 250 g/L, 275 g/L, 300 g/L, 325 g/L, 350 g/L, 375 g/L, 400 g/L, 425 g/L, 450 g/L, 475 g/L, or 500 g/L.

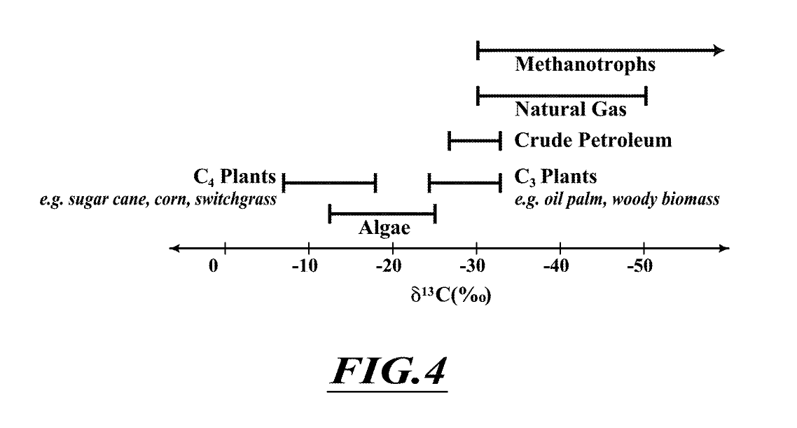

Isoprene produced using the compositions and methods provided herein may be distinguished from isoprene produced from petrochemicals or from isoprene biosynthesized from non-methanotrophic bacteria by carbon finger-printing. By way of background, stable isotopic measurements and mass balance approaches are widely used to evaluate global sources and sinks of methane (see Whiticar and Faber, Org. Geochem. 10:759, 1986; Whiticar, Org. Geochem. 16: 531, 1990). A measure of the degree of carbon isotopic fractionation caused by microbial oxidation of methane can be determined by measuring the isotopic signature (i.e., ratio of stable isotopes ¹³C:¹²C) value of the residual methane. For example, aerobic methanotrophs can metabolize methane through a specific enzyme, methane monoxygenase (MMO). Methanotrophs convert methane to methanol and subsequently formaldehyde. Formaldehyde can be further oxidized to CO₂ to provide energy to the cell in the form of reducing equivalents (NADH), or incorporated into biomass through either the RuMP or serine cycles (Hanson and Hanson, Microbiol. Rev. 60:439, 1996), which are directly analogous to carbon assimilation pathways in photosynthetic organisms. More specifically, a Type I methanotroph uses the RuMP pathway for biomass synthesis and generates biomass entirely from CH₄, whereas a Type II methanotroph uses the serine pathway that assimilates 50-70% of the cell carbon from CH₄ and 30-50% from CO₂ (Hanson and Hanson, 1996). Methods for measuring carbon isotope compositions are provided in, for example, Templeton et al. (Geochim. Cosmochim. Acta 70:1739, 2006), which methods are hereby incorporated by reference in their entirety. The ¹³C/¹²C stable carbon isotope ratio of isoprene (reported as a δ¹³C value in parts per thousand, ‰), varies depending on the source and purity of the C₁ substrate used (see, e.g., FIG. 4).

For example, isoprene derived from petroleum has a δ¹³C distribution of about −22‰ to about −24‰. Isoprene biosynthesized primarily from corn-derived glucose (δ¹³C −10.73‰) has a δ¹³C of about −14.66‰ to −14.85‰. Isoprene biosynthesized from renewable carbon sources are expected to have δ¹³C values that are less negative than isoprene derived from petroleum. However, the δ¹³C distribution of methane from natural gas is differentiated from most carbon sources, with a more negative δ¹³C distribution than crude petroleum. Methanotrophic bacteria display a preference for utilizing ¹²C and reducing their intake of ¹³C under conditions of excess methane, resulting in further negative shifting of the δ¹³C value. Isoprene produced by methanotrophic bacteria as described herein has a δ¹³C distribution more negative than isoprene from crude petroleum or renewable carbon sources, ranging from about −30‰ to about −50‰. In certain embodiments, an isoprene composition has a δ¹³C distribution of less than about −30‰, −40‰, or −50‰. In certain embodiments, an isoprene composition has a δ¹³C distribution from about −30‰ to about −40‰, or from about −40‰ to about −50‰.

In certain embodiments, an isoprene composition has a δ¹³C distribution of less than about −30‰, −40‰, or −50‰. In certain embodiments, an isoprene composition has a δ¹³C distribution from about −30‰ to about −40‰, or from about −40‰ to about −50‰. In further embodiments, an isoprene composition has a δ¹³C of less than −30‰, less than −31‰, less than −32‰, less than −33‰, less than −34‰, less than −35‰, less than −36‰, less than −37‰, less than −38‰, less than −39‰, less than −40‰, less than −41‰, less than −42‰, less than −43‰, less than −44‰, less than −45‰, less than −46‰, less than −47‰, less than −48‰, less than −49‰, less than −50‰, less than −51‰, less than −52‰, less than −53‰, less than −54‰, less than −55‰, less than −56‰, less than −57‰, less than −58‰, less than −59‰, less than −60‰, less than −61‰, less than −62‰, less than −63‰, less than −64‰, less than −65‰, less than −66‰, less than −67‰, less than −68‰, less than −69‰, or less than −70‰.

### Measuring Isoprene Production

Isoprene production may be may be measured using methods known in the art. For example, samples from the off-gas of the fermenter gas may be analyzed by gas chromatography, equipped with a flame ionization detector and a column selected to detect short-chain hydrocarbons (Lindberg et al., 2010, Metabolic Eng. 12:70-79). Amounts of isoprene produced may be estimated by comparison with a pure isoprene standard. Silver et al., J. Biol. Chem. 270:13010, 1995, U.S. Pat. No. 5,849,970, and references cited therein, describe methods for measuring isoprene production using gas chromatography with a mercuric oxide gas detector, which methods are hereby incorporated by reference in their entirety.

### Recovery and Purification of Isoprene

In certain embodiments, any of the methods described herein may further comprise recovering or purifying isoprene produced by the host methanotrophic bacteria. While the exemplary recovery and purification methods described below refer to isoprene, they may also be applied to isoprenoid or other compounds derived from isoprene.

Isoprene produced using the compositions and methods provided in the present disclosure may be recovered from fermentation systems by bubbling a gas stream (e.g., nitrogen, air) through a culture of isoprene-producing methanotrophs. Methods of altering gas-sparging rates of fermentation medium to enhance concentration of isoprene in the fermentation off-gas are known in the art. Isoprene is further recovered and purified using techniques known in the art, such as gas stripping, distillation, polymer membrane enhanced separation, fractionation, pervaporation, adsorption/desorption (e.g., silica gel, carbon cartridges), thermal or vacuum desorption of isoprene from a solid phase, or extraction of isoprene immobilized or adsorbed to a solid phase with a solvent (see, e.g., U.S. Pat. No. 4,703,007, U.S. Pat. No. 4,570,029, U.S. Pat. No. 4,147,848, U.S. Pat. No. 5,035,794, PCT Publication WO2011/075534, the methods from each of which are hereby incorporated by reference in their entireties). Extractive distillation with an alcohol (e.g., ethanol, methanol, propanol, or a combination thereof) may be used to recover isoprene. Isoprene recovery may involve isolation of isoprene in liquid form (e.g., neat solution of isoprene or solution of isoprene with a solvent). Recovery of isoprene in gaseous form may involve gas stripping, where isoprene vapor from the fermentation off-gas is removed in a continuous manner. Gas stripping may be achieved using a variety of methods, including for example, adsorption to a solid phase, partition into a liquid phase, or direct condensation. Membrane enrichment of a dilute isoprene vapor stream above the dew point of the vapor may also be used to condense liquid isoprene. Isoprene gas may also be compressed and condensed.

Recovery and purification of isoprene may comprise one step or multiple steps. Recovery and purification methods may be used individually or in combination to obtain high purity isoprene. In some embodiments, removal of isoprene gas from the fermentation off-gas and conversion to a liquid phase are performed simultaneously. For example, isoprene may be directly condensed from an off-gas stream into a liquid. In other embodiments, removal of isoprene gas from the fermentation off-gas and conversion to a liquid phase are performed sequentially (e.g., isoprene may be adsorbed to a solid phase and then extracted with a solvent).

In certain embodiments, isoprene recovered from a culture system using the compositions and methods described herein undergoes further purification (e.g., separation from one or more non-isoprene components that are present in the isoprene liquid or vapor during isoprene production). In certain embodiments, isoprene is a substantially purified liquid. Purification methods are known in the art, and include extractive distillation and chromatography, and purity may be assessed by methods such as column chromatography, HPLC, or GC-MS analysis. In certain embodiments, isoprene has at least 20%, 25%, 30%, 35%, 40%, 45%, 50%, 55%, 60%, 65%, 70%, 75%, 80%, 85%, 90%, 95%, or 99% purity by weight.

In certain embodiments, at least a portion of the gas phase that remains after one or more steps of isoprene recovery is recycled back into the fermentation system.

### Further Processing of Isoprene

Isoprene produced using the compositions and methods described herein may be further processed into other high value products using methods known in the art. After recovery or purification, isoprene may be polymerized using various catalysts to form various polyisoprene isomers (Senyek, “Isoprene Polymers”, Encyclopedia of Polymer Science and Technology, 2002, John Wiley & Sons, Inc.). Isoprene may also be polymerized with styrene or butadiene to form various elastomers. Photochemical polymerization of isoprene initiated by hydrogen peroxide forms hydroxyl terminated polyisoprene, which can be used as a pressure-sensitive adhesive. Isoprene telomerization products are also useful as fuels (Clement et al., 2008, Chem. Eur. J. 14:7408-7420; Jackstell et al., 2007, J. Organometallic Chem. 692:4737-4744). Isoprene may also be chemically modified into dimer (10-carbon) and trimer (15-carbon) hydrocarbon alkenes using catalysts (Clement et al., 2008, Chem. Eur. J. 14:7408-7420; Gordillo et al., 2009, Adv. Synth. Catal. 351:325-330). Alkenes may be hydrogenated to form long-chain branched alkanes, which may be used as fuels or solvents. Isoprene may be converted into isoprenoid compounds, such as terpenes, ginkgolides, sterols, or carotenoids. Isoprene may also be converted into isoprenoid-based biofuels, such as farnesane, bisabolane, pinene, isopentanol, or any combination thereof (Peralta-Yahya et al., 2012, Nature 488:320-328).

Methods of Screening for Mutants with Increased Isoprene Pathway Precursors

Genome or gene specific mutations may be induced in host methanotrophic bacteria in an effort to improve production of isoprene precursors. Methods to elicit genomic mutations are known in the art (see, e.g., Thomas D. Brock, Biotechnology: A Textbook of Industrial Microbiology, 2^{nd} Ed. (1989) Sinauer Associates, Inc., Sunderland, Mass.; Deshpande, 1992, Appl. Biochem. Biotechnol. 36:227) and include for example, UV irradiation, chemical mutagenesis (e.g., acridine dyes, HNO₂, NH₂OH), and transposon mutagenesis (e.g., Ty1, Tn7, Tn5). Random mutagenesis techniques, for example error-prone PCR, rolling circle error-prone PCR, or mutator strains, may be used to create random mutant libraries of specific genes or gene sets. Site directed mutagenesis may be also be used to create mutant libraries of specific genes or gene sets.

The present disclosure provides methods for screening mutant methanotrophic strains with improved production of isoprene precursors by engineering a lycopene pathway into methanotrophic bacteria. Lycopene and isoprene synthesis pathways use the same universal precursors, isopentenyl diphosphate (IPP) and dimethylallyl diphosphate (DMAPP) (see FIGS. 1 and 6); lycopene and isoprene biosynthesis share most of the DXP pathway. Beneficial genome mutations that result in improved lycoprene production, as measured by increased red pigmentation of the bacteria, may also result in improved isoprene synthesis by increasing IPP and DMAPP production if the mutations affect overlapping pathway components.

In certain embodiments, methods for screening mutant methanotrophic bacteria comprise: (a) exposing methanotrophic bacteria to a mutagen to produce mutant methanotrophic bacteria; (b) transforming the mutant methanotrophic bacteria with exogenous nucleic acids encoding geranylgeranyl diphosphate synthase (GGPPS), phytoene synthase (CRTB), and phytoene dehydrogenase (CRTI); and (c) culturing the mutant methanotrophic bacteria under conditions sufficient for growth; wherein a mutant methanotrophic bacterium that exhibits an increase in red pigmentation as compared to a reference methanotrophic bacterium that has been transformed with GGPPS, CRTB and CRTI and has not been exposed to a mutagen indicates that the mutant methanotrophic bacterium with increased red pigmentation exhibits increased synthesis of isoprene precursors as compared to the reference methanotrophic bacterium. In certain embodiments, an isoprene precursor is IPP or DMAPP. In some embodiments, the mutagen is a radiation, a chemical, a plasmid, or a transposon. Mutant methanotrophic bacteria identified as having increased isoprene precursor production via increased lycopene pathway activity may then be engineered with isoprene biosynthetic pathways as described herein. In some embodiments, the mutant methanotrophic bacterium with increased red pigmentation or a clonal cell thereof is transformed with an exogenous nucleic acid encoding an isoprene synthase (e.g., IspS). In certain embodiments, at least one, two, or all of the lycopene pathway genes (GGPPS, CRTB, and CRTI) are removed or inactivated from the mutant methanotrophic bacteria identified as having increased isoprene precursor production before or after being transformed with a nucleic acid encoding IspS. Co-expression of a functional lycopene pathway with a functional isoprene pathway would compete for shared precursors DMAPP and IPP, and may lower isoprene production. Isoprene production in the mutant methanotrophic bacterium identified via the screening methods described herein may then be compared with a reference methanotrophic bacterium having isoprene biosynthetic capability to confirm increased isoprene levels. It is apparent to one of skill in the art that clonal bacterial stocks may be saved at each step during the method for subsequent use. For example, for a particular bacterium that has been identified as having increased red pigmentation, a clonal stock of that bacterium saved prior to transformation with the lycopene pathway (i.e., a bacterium with a potentially beneficial mutation for isoprene synthesis as identified by lycopene screening but without the exogenous lycopene pathway) may be transformed with an isoprene synthase (e.g., IspS).

Also provided in the present disclosure are methods for screening isoprene pathway genes in methanotrophic bacteria. These screening methods may be used to identify isoprene pathway genes that result in increased synthesis of isoprene precursors DMAPP and IPP by engineering a lycopene pathway into the methanotrophic bacteria as a colorimetric readout. Lycopene and isoprene synthesis pathways use the same universal precursors, IPP and DMAPP (see FIGS. 1 and 6). Methanotrophic bacteria may be modified with heterologous isoprene pathway genes, overexpression of homologous isoprene pathway genes, variant isoprene pathway genes, or any combination thereof to identify bacteria with improved lycopene production, as measured by increased red pigmentation of the bacteria. Bacteria identified as having increased lycopene production may also exhibit improved isoprene synthesis because of increased IPP and DMAPP production.

In certain embodiments, methods for screening isoprene pathway genes in methanotrophic bacteria comprise: (a) transforming the methanotrophic bacteria with (i) at least one exogenous nucleic acid encoding an isoprene pathway enzyme; (ii) exogenous nucleic acids encoding geranylgeranyl disphosphate synthase (GGPPS), phytoene synthase (CRTB), and phytoene dehydrogenase (CRTI); and (b) culturing the methanotrophic bacteria from step (a) under conditions sufficient for growth; wherein the transformed methanotrophic bacterium that exhibits an increase in red pigmentation as compared to a reference methanotrophic bacterium that has been transformed with exogenous nucleic acids encoding GGPPS, CRTB, and CRTI and does not contain the at least one exogenous nucleic acid encoding an isoprene pathway enzyme indicates that the at least one exogenous nucleic acid encoding an isoprene pathway enzyme confers increased isoprene precursor synthesis as compared to the reference methanotrophic bacterium. In certain embodiments, the isoprene pathway enzyme is a DXP pathway enzyme (e.g., DXS, DXR, IspD, IspE, IspF, IspG, IspH, or IDI) or a mevalonate pathway enzyme (e.g., AACT, HMGS, HMGR, MK, PMK, MPD, or IDI). The at least one exogenous nucleic acid encoding an isoprene pathway enzyme may be a heterologous nucleic acid or a homologous nucleic acid. The heterologous nucleic acid may be codon optimized for expression in the host methanotrophic bacteria. In some embodiments, the homologous nucleic acid is overexpressed in the methanotrophic bacteria. In the various embodiments described herein, the at least one exogenous nucleic acid encoding an isoprene pathway enzyme may be a non-naturally occurring variant. The non-naturally occurring variant may be generated by random mutagenesis, site-directed mutagenesis, or synthesized (in whole or in part). In certain embodiments, the non-naturally occurring variant comprises at least one amino acid substitution as compared to a reference nucleic acid encoding an isoprene pathway enzyme.

Sources of lycopene pathway enzymes are known in the art and may be any organism that naturally possesses a lycopene pathway, including species of plants, photosynthetic bacteria, fungi, and algae. Examples of nucleic acid sequences for geranylgeranyl diphosphate synthase available in the NCBI database include Accession Nos: AB000835 (Arabidopsis thaliana); AB016043 (Homo sapiens); AB019036 (Homo sapiens); AB016044 (Mus musculus); AB027705 (Dacus carota); AB034249 (Croton sublyratus); AB034250 (Scoparia dulcis); AF049659 (Drosophila melanogaster); AF139916 (Brevibacterium linens); AF279807 (Penicillum paxilli); AJ010302 (Rhodobacter sphaeroides); AJ133724 (Mycobacterium aurum); L25813 (Arabidopsis thaliana); U44876 (Arabidopsis thaliana); and U15778 (Lupinus albus). Examples of nucleic acid sequences for phytoene synthase available in the NCBI database include Accession Nos: AB001284 (Spirulina platensis); AB032797 (Daucus carota); AB034704 (Rubrivivax gelatinosus); AB037975 (Citrus unshui); AF009954 (Arabidopsis thaliana); AF139916 (Brevibacterium linens); AF152892 (Citrus×paradise); AF218415 (Bradyrhizobium sp. ORS278); AF220218 (Citrus unshiu); AJ133724 (Mycobacterium aurum); and AJ304825 (Helianthus annuus). Examples of nucleic acid sequences for phytoene dehydrogenase available in the NCBI database include Accession Nos: AB046992 (Citrus unshiu); AF139916 (Brevibacterium linens); AF218415 (Bradyrhizobium sp. ORS278); AF251014 (Tagetes erecta); L16237 (Arabidopsis thaliana); L39266 (Zea mays); M64704 (Glycine max); AF364515 (Citrus×paradisi); D83514 (Erythrobacter longus); M88683 (Lycopersicon esculentum); and X55289 (Synechococcus).

### EXAMPLES

### Example 1

### Cloning and Expression of Isoprene Synthase in Methanococcus Capsulatus Bath Strain

To create isoprene producing methanotrophic strains, a methanotroph expression vector containing a gene encoding isoprene synthase (IspS) was inserted into the Methylococcus capsulatus Bath, Methylosinus trichosporium OB3b, and Methylomonas sp. 16A via conjugative mating. An episomal expression plasmid (containing sequences encoding origin of replication, origin of transfer, drug resistance marker (kanamycin), and multiple cloning sites), was used to clone either a codon optimized Salix sp. IspS polynucleotide sequence (SEQ ID NO:19 for Methylococcus capsulatus Bath) downstream of a methanol dehydrogenase (MDH) promoter, or a Pueraria montana codon optimized IspS polynucleotide sequence (with the amino-terminal chloroplast targeting sequence removed) (SEQ ID NO:17 for Methylococcus capsulatus Bath) downstream of an IPTG-inducible (LacIq) promoter. Colonies of E. coli strain containing the IspS harboring plasmid (donor strain) and the E. coli containing pRK2013 plasmid (ATCC) (helper strain) were inoculated in liquid LB containing Kanamycin (30 μg/mL) and grown at 37° C. overnight. One part of each liquid donor culture and helper culture was inoculated into 100 parts of fresh LB containing Kanamycin (30 μg/mL) for 3-5 h before they were used to mate with the recipient methanotrophic strains. Methanotrophic (recipient) strains were inoculated in liquid MM-W1 medium (Pieja et al., 2011, Microbial Ecology 62:564-573) with about 40 mL methane for 1-2 days prior to mating until they reached logarithmic growth phase (OD600 of about 0.3).

Triparental mating was conducted by preparing the recipient, donor, and helper strain at a volume so that the OD600 ratio was 2:1:1 (e.g., 1 mL of methanotroph with an OD600 of 1.5, 1 mL of donor with an OD600 of 0.75, and 1 mL of helper with an OD600 of 0.75). These cells were then harvested by centrifugation at 5,300 rpm for 7 mins. at 25° C. The supernatant was removed, and the cell pellets were gently resuspended in 500 μL MM-W1. For E. coli donor and helper strains, centrifugation and resuspension were repeated 2 more times to ensure the removal of antibiotics. An equal volume of the resuspended cells of recipient, donor, and helper strains were then combined and mixed by gentle pipetting. The mating composition was spun down for 30-60 s at 13.2 k rpm, and the supernatant was removed as much as possible. The cell pellet was then gently mixed and deposited as a single droplet onto mating agar (complete MM-W1 medium containing sterile 0.5% yeast extract). The mating plates were incubated for 48 h in an oxoid chamber containing methane and air at 30° C. in the case of using Methylosinus trichosporium OB3b or Methylomonas sp. 16a as the recipient, or at 37° C. in the case of using Methylococcus capsulatus Bath as the recipient strain. After the 48 h incubation period, the cells from the mating plates were collected by adding 1 mL MM-W1 medium onto the plates and transferring the suspended cells to a 2 mL Eppendorf tube. The cells were pelleted by centrifugation and resuspended with 100 μL fresh MM-W1 before plating onto selection plates (complete MM-W1 agar medium containing kanamycin 10 μg/mL) to select for cells that stably maintain the constructs. Plasmid bearing methanotrophs appeared on these plates after about 1 week of incubation at 42° C. for Methylococcus capsulatus Bath strain or 1 week of incubation at 30° C. for Methylomonas 16a and Methylosinus trichosporium OB3b in an oxoid chamber containing methane-air mixture. Methylococcus capsulatus Bath strain clones were then cultured in 1 mL liquid media and analyzed for isoprene production.

### Example 2

### Production of Isoprene by Methanococcus Capsulatus Bath Strain

Headspace gas samples (250 μl) from enclosed 5 mL cultures grown overnight of M. capsulatus Bath strain containing either a vector containing constitutive MDH promoter-Salix sp. IspS or a vector containing an IPTG-inducible (LacIq) promoter-Pueraria montana IspS (grown in the presence or absence of 0.1-10 mM IPTG) were obtained. Gas samples were injected onto a gas chromatograph with flame ionization detector (Hewlett Packard 5890). Chromatography conditions include an Agilent CP-PoraBOND U (25 m×0.32 mm i.d.) column, oven program 50° C., 1.5 min; 25° C., 1 min; 300° C., 10 min. The eluted peak was detected by flame ionization and integrated peaks were quantitated by comparison to isoprene standard (pure isoprene dissolved in deionized water).

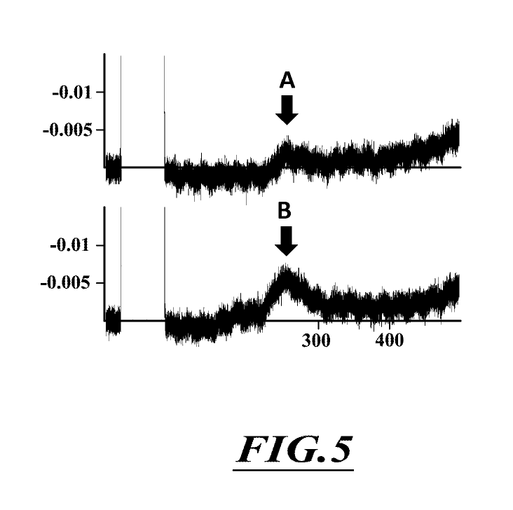

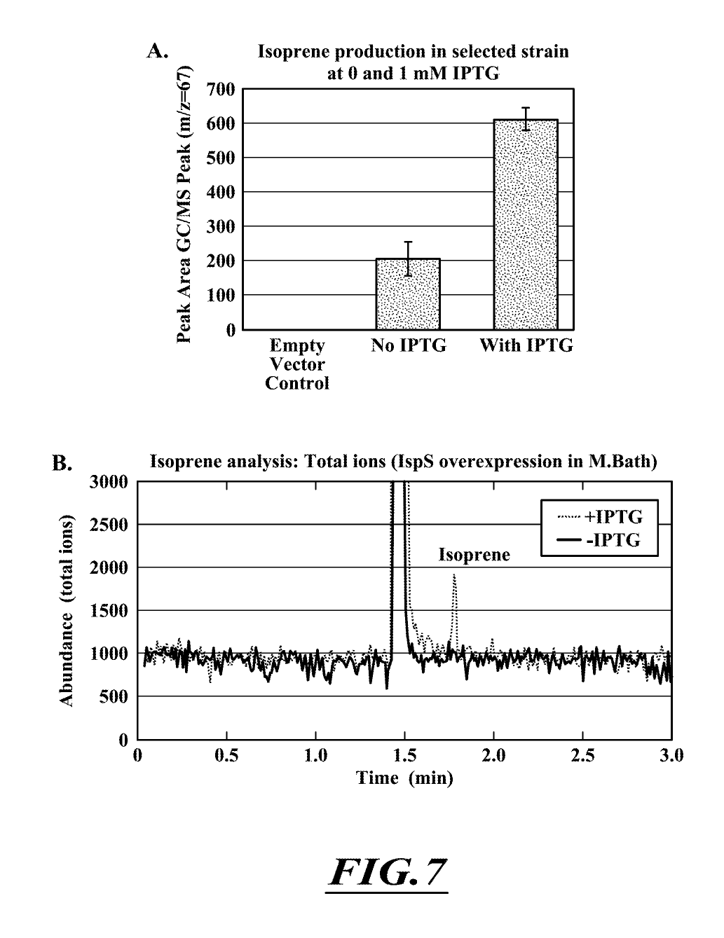

M. capsulatus Bath produced more isoprene when expressing the Pueraria montana IspS as compared to expression of the Salix sp. IspS. In addition, and the amount of isoprene produced in M. capsulatus Bath expressing Pueraria montana IspS directly correlated with induction of the LacIq promoter with IPTG (see FIG. 7A). FIGS. 5 and 7B show the GC/MS chromatography of headspace samples from the Salix sp. and Pueraria montana variant samples, respectively. In FIG. 5, Sample A is a negative control showing the background signal from headspace from untransformed cells. The isoprene yield in sample B of FIG. 5 was about 10 mg/L. FIG. 7B shows a substantial amount of isoprene being produced.

### Example 3

### Engineering a DXP Pathway with Improved Isoprene Production

Random mutations are introduced in the DXP pathway operon (i.e., DXS-DXR-IspD-IspE-IspF-IspG-IspH) for the purpose of generating novel gene sequences or regulatory elements within the pathway that overall, result in an improvement of enzymes for synthesis of the committed precursors of isoprene (IPP and DMAPP). To construct a facile high-throughput screening method for isolating an improved DXP pathway, a lycopene synthesis pathway comprising ggpps, crtB and crtI was utilized as a colorimetric reporter. A random mutagenesis library of the DXP pathway is created by error-prone PCR at low, medium, and high mutation rate using GENEMORPH® II random mutagenesis kit (Stratagene). The library is then cloned into a methanotrophic expression plasmid containing ggpps, crtB, and crtI gene sequences, whereby their polycistronic expression is driven by a strong methanotroph promoter sequence (e.g., methanol dehydrogenase promoter). A pool of the library containing plasmid is then isolated from more than approximately 10⁶ transformants of E. coli DH10B. The plasmid library is then used to transform a methanotrophic strain. Colonies that display bright red coloration are isolated after an extended incubation period (as visualized on MM-WI plates). Following plasmid extraction and sequencing, the mutant DXP pathway genes are used as a pool in the next round of error-prone PCR. The methanotroph strain containing the wild-type DXP pathway genes, together with the plasmid containing ggpps, crtB, crtI, serves as a baseline comparison of lycopene formation for isolating mutant DXP pathway genes. The iteration of mutation and screening is stopped after no additional colony displaying increased red coloration is identified. The plasmids harboring the novel DXP pathway genes are then isolated from the methanotroph host. These novel DXP pathway genes are then coexpressed with IspS in methanotrophic host bacteria to confirm improvement of isoprene production.

### Example 4

### Stable Carbon Isotope Distribution in C₁ Metabolizing Microorganisms

Dry samples of M. trichosporium biomass were analyzed for carbon and nitrogen content (% dry weight), and carbon (¹³C) and nitrogen (¹⁵N) stable isotope ratios via elemental analyzer/continuous flow isotope ratio mass spectrometry using a CHNOS Elemental Analyzer (vario ISOTOPE cube, Elementar, Hanau, Germany) coupled with an IsoPrime100 IRMS (Isoprime, Cheadle, UK). Samples of methanotrophic biomass cultured in fermenters or serum bottles were centrifuged, resuspended in deionized water and volumes corresponding to 0.2-2 mg carbon (about 0.5-5 mg dry cell weight) were transferred to 5×9 mm tin capsules (Costech Analytical Technologies, Inc., Valencia, Calif.) and dried at 80° C. for 24 hours. Standards containing 0.1 mg carbon provided reliable δ¹³C values.

The isotope ratio is expressed in “delta” notation (‰), wherein the isotopic composition of a material relative to that of a standard on a per million deviation basis is given by δ¹³C (or δ¹⁵N)=(R_{Sample}/R_{Standard-1})×1,000, wherein R is the molecular ratio of heavy to light isotope forms. The standard for carbon is the Vienna Pee Dee Belemnite (V-PDB) and for nitrogen is air. The NIST (National Institute of Standards and Technology) proposed SRM (Standard Reference Material) No. 1547, peach leaves, was used as a calibration standard. All isotope analyses were conducted at the Center for Stable Isotope Biogeochemistry at the University of California, Berkeley. Long-term external precision for C and N isotope analyses is 0.10% and 0.15%, respectively.

M. trichosporium strain OB3b was grown on methane in three different fermentation batches, M. capsulatus Bath was grown on methane in two different fermentation batches, and Methylomonas sp. 16a was grown on methane in a single fermentation batch. The biomass from each of these cultures was analyzed for stable carbon isotope distribution (δ¹³C values; see Table 4).

TABLE 4
Stable Carbon Isotope Distribution in Different Methanotrophs

| Methanotroph | Batch No. | EFT (h)† | OD₆₀₀ | DCW* | δ¹³C Cells |
|---|---|---|---|---|---|
| Mt OB3b | 68A | 48 | 1.80 | 1.00 | −57.9 |
| | | 64 | 1.97 | 1.10 | −57.8 |
| | | 71 | 2.10 | 1.17 | −58.0 |
| | | 88 | 3.10 | 1.73 | −58.1 |
| | | 97 | 4.30 | 2.40 | −57.8 |
| | | 113 | 6.00 | 3.35 | −57.0 |
| | | 127 | 8.40 | 4.69 | −56.3 |
| Mt OB3b | 68B | 32 | 2.90 | 1.62 | −58.3 |
| | | 41 | 4.60 | 2.57 | −58.4 |
| | | 47 | 5.89 | 3.29 | −58.0 |
| | | 56 | 7.90 | 4.41 | −57.5 |
| Mt OB3b | 68C | 72 | 5.32 | 2.97 | −57.9 |
| | | 79.5 | 5.90 | 3.29 | −58.0 |
| | | 88 | 5.60 | 3.12 | −57.8 |
| | | 94 | 5.62 | 3.14 | −57.7 |
| Mc Bath | 62B | 10 | 2.47 | 0.88 | −59.9 |
| | | 17.5 | 5.80 | 2.06 | −61.0 |
| | | 20 | 7.32 | 2.60 | −61.1 |
| | | 23 | 9.34 | 3.32 | −60.8 |
| | | 26 | 10.30 | 3.66 | −60.1 |
| Mc Bath | 62A | 10 | 2.95 | 1.05 | −55.9 |
| | | 13.5 | 3.59 | 1.27 | −56.8 |
| | | 17.5 | 5.40 | 1.92 | −55.2 |
| | | 23 | 6.08 | 2.16 | −57.2 |
| | | 26 | 6.26 | 2.22 | −57.6 |
| Mms 16a | 66B | 16 | 2.13 | 0.89 | −65.5 |
| | | 18 | 2.59 | 1.09 | −65.1 |
| | | 20.3 | 3.62 | 1.52 | −65.5 |
| | | 27 | 5.50 | 2.31 | −66.2 |
| | | 40.5 | 9.80 | 4.12 | −66.3 |

*DCW, Dry Cell Weight is reported in g/L calculated from the measured optical densities (OD₆₀₀) using specific correlation factors relating OD of 1.0 to 0.558 g/L for Mt OB3b, OD of 1.0 to 0.355 g/L for Mc Bath, and OD of 1.0 to 0.42 g/L for Mms 16a. For Mt OB3b, the initial concentration of bicarbonate used per fermentation was 1.2 mM or 0.01% (Batch No. 68C) and 0.1% or 12 mM (Batch Nos. 68A and 68B).
†EFT = effective fermentation time in hours

### Example 5

### Effect of Methane Source and Purity on Stable Carbon Isotope Distribution

To examine methanotroph growth on methane containing natural gas components, a series of 0.5-liter serum bottles containing 100 mL defined media MMS 1.0 were inoculated with Methylosinus trichosporium OB3b or Methylococcus capsulatus Bath from a serum bottle batch culture (5% v/v) grown in the same media supplied with a 1:1 (v/v) mixture of methane and air. The composition of medium MMS1.0 was as follows: 0.8 mM MgSO₄*7H₂O, 30 mM NaNO₃, 0.14 mM CaCl₂, 1.2 mM NaHCO₃, 2.35 mM KH₂PO₄, 3.4 mM K₂HPO₄, 20.7 μM Na₂MoO₄*2H₂O, 6 μM CuSO₄*5H₂O, 10 μM Fe^{III}—Na-EDTA, and 1 mL per liter of a trace metals solution (containing, per L: 500 mg FeSO4*7H₂O, 400 mg ZnSO₄*7H₂O, 20 mg MnCl₂*7H2O, 50 mg CoCl₂*6H₂O, 10 mg NiCl₂*6H₂O, 15 mg H₃BO₃, 250 mg EDTA). Phosphate, bicarbonate, and Fe^{III}—Na-EDTA were added after media was autoclaved and cooled. The final pH of the media was 7.0±0.1.

The inoculated bottles were sealed with rubber sleeve stoppers and injected with 60 mL methane gas added via syringe through sterile 0.45 μm filter and sterile 27 G needles. Duplicate cultures were each injected with 60 mL volumes of (A) methane of 99% purity (grade 2.0, Praxair through Alliance Gas, San Carlos, Calif.), (B) methane of 70% purity representing a natural gas standard (Sigma-Aldrich; also containing 9% ethane, 6% propane, 3% methylpropane, 3% butane, and other minor hydrocarbon components), (C) methane of 85% purity delivered as a 1:1 mixture of methane sources A and B; and (D)>93% methane (grade 1.3, Specialty Chemical Products, South Houston, Tex.; in-house analysis showed composition>99% methane). The cultures were incubated at 30° C. (M. trichosporium strain OB3b) or 42° C. (M. capsulatus Bath) with rotary shaking at 250 rpm and growth was measured at approximately 12 hour intervals by withdrawing 1 mL samples to determine OD₆₀₀. At these times, the bottles were vented and headspace replaced with 60 mL of the respective methane source (A, B, C, or D) and 60 mL of concentrated oxygen (at least 85% purity). At about 24 hour intervals, 5 mL samples were removed, cells recovered by centrifugation (8,000 rpm, 10 minutes), and then stored at −80° C. before analysis.

Analysis of carbon and nitrogen content (% dry weight), and carbon (¹³C) and nitrogen (¹⁵N) stable isotope ratios, for methanotrophic biomass derived from M. trichosporium strain OB3b and M. capsulatus Bath were carried out as described in Example 4. Table 5 shows the results of stable carbon isotope analysis for biomass samples from M. capsulatus Bath grown on methane having different levels of purity and in various batches of bottle cultures.

TABLE 5
Stable Carbon Isotope Distribution of M. capsulatus Bath Grown
on Different Methane Sources having Different Purity

| | | Time | | | |
|---|---|---|---|---|---|
| Methane* | Batch No. | (h)† | OD₆₀₀ | DCW (g/L) | δ¹³C Cells |
| A | 62C | 22 | 1.02 | 0.36 | −40.3 |
| | | 56 | 2.01 | 0.71 | −41.7 |
| | | 73 | 2.31 | 0.82 | −42.5 |
| | 62D | 22 | 1.14 | 0.40 | −39.3 |
| | | 56 | 2.07 | 0.73 | −41.6 |
| | | 73 | 2.39 | 0.85 | −42.0 |
| B | 62E | 22 | 0.47 | 0.17 | −44.7 |
| | | 56 | 0.49 | 0.17 | −45.4 |
| | | 73 | 0.29 | 0.10 | −45.4 |
| | 62F | 22 | 0.62 | 0.22 | −42.3 |
| | | 56 | 0.63 | 0.22 | −43.6 |
| | | 73 | 0.30 | 0.11 | −43.7 |
| C | 62G | 22 | 0.70 | 0.25 | −40.7 |
| | | 56 | 1.14 | 0.40 | −44.8 |
| | | 73 | 1.36 | 0.48 | −45.8 |
| | 62H | 22 | 0.62 | 0.22 | −40.9 |
| | | 56 | 1.03 | 0.37 | −44.7 |
| | | 73 | 1.23 | 0.44 | −45.9 |

*Methane purity: A: 99% methane, grade 2.0 (min. 99%); B: 70% methane, natural gas standard (contains 9% ethane, 6% propane, 3% methylpropane, 3% butane); C: 85% methane (1:1 mix of A and B methane)
†Time = bottle culture time in hours

The average δ¹³C for M. capsulatus Bath grown on one source of methane (A, 99%) was −41.2±1.2, while the average δ¹³C for M. capsulatus Bath grown on a different source of methane (B, 70%) was −44.2±1.2. When methane sources A and B were mixed, an intermediate average δ¹³C of −43.8±2.4 was observed. These data show that the δ¹³C of cell material grown on methane sources A and B are significantly different from each other due to the differences in the δ¹³C of the input methane. But, cells grown on a mixture of the two gasses preferentially utilize ¹²C and, therefore, show a trend to more negative δ¹³C values.

A similar experiment was performed to examine whether two different methanotrophs, Methylococcus capsulatus Bath and Methylosinus trichosporium OB3b, grown on different methane sources and in various batches of bottle cultures showed a difference in δ¹³C distribution (see Table 6).

TABLE 6
Stable Carbon Isotope Distribution of Different Methanotrophs
Grown on Different Methane Sources of Different Purity

| | | Batch | Time | | DCW | δ¹³C |
|---|---|---|---|---|---|---|
| Strain | Methane* | No. | (h)† | OD₆₀₀ | (g/L) | Cells |
| Mc | A | 62I | 18 | 0.494 | 0.18 | −54.3 |
| Bath | | | 40 | 2.33 | 0.83 | −42.1 |
| | | | 48 | 3.08 | 1.09 | −37.1 |
| Mc | D | 62J | 18 | 0.592 | 0.21 | −38.3 |
| Bath | | | 40 | 1.93 | 0.69 | −37.8 |
| | | | 48 | 2.5 | 0.89 | −37.8 |
| Mc | D | 62K | 18 | 0.564 | 0.20 | −38.6 |
| Bath | | | 40 | 1.53 | 0.54 | −37.5 |
| | | | 48 | 2.19 | 0.78 | −37.6 |
| Mt | A | 68D | 118 | 0.422 | 0.24 | −50.2 |
| OB3b | | | 137 | 0.99 | 0.55 | −47.7 |
| | | | 162 | 1.43 | 0.80 | −45.9 |
| Mt | A | 68E | 118 | 0.474 | 0.26 | −49.9 |
| OB3b | | | 137 | 1.065 | 0.59 | −47.6 |
| | | | 162 | 1.51 | 0.84 | −45.2 |
| Mt | D | 68F | 118 | 0.534 | 0.30 | −45.6 |
| OB3b | | | 137 | 1.119 | 0.62 | −38.7 |
| | | | 162 | 1.63 | 0.91 | −36.4 |
| Mt | D | 68G | 118 | 0.544 | 0.30 | −44.8 |
| OB3b | | | 137 | 1.131 | 0.63 | −39.1 |
| | | | 162 | 1.6 | 0.89 | −34.2 |

*Methane sources and purity: A: 99% methane (grade 2.0); D: >93% methane (grade 1.3)
†Time = bottle culture time in hours

The average δ¹³C for M. capsulatus grown on a first methane source (A) was −44.5±8.8, while the average δ¹³C for M. trichosporium was −47.8±2.0 grown on the same methane source. The average δ¹³C for M. capsulatus grown on the second methane source (B) was −37.9±0.4, while the average δ¹³C for M. trichosporium was −39.8±4.5. These data show that the δ¹³C of cell material grown on a methane source is highly similar to the δ¹³C of cell material from a different strain grown on the same source of methane. Thus, the observed δ¹³C of cell material appears to be primarily dependent on the composition of the input gas rather than a property of a particular bacterial strain being studied.

The various embodiments described above can be combined to provide further embodiments. All of the U.S. patents, U.S. patent application publications, U.S. patent applications, foreign patents, foreign patent applications and non-patent publications referred to in this specification or listed in the Application Data Sheet, including but not limited to U.S. Patent Application No. 61/774,342 and U.S. Patent Application No. 61/928,333 are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A genetically engineered methanotrophic bacterium, comprising an exogenous nucleic acid molecule encoding an isoprene synthase, wherein the methanotrophic bacterium is capable of converting a carbon feedstock into isoprene.

2. The genetically engineered methanotrophic bacterium of claim 1, wherein the nucleic acid molecule encoding the isoprene synthase is an isoprene synthase of Populus alba, Populus trichocarpa, Populus tremuloides, Populus nigra, Populus alba×Populus tremula, Populus×canescens, Pueraria montana, Pueraria lobata, Quercus robur, Faboideae, Salix discolor, Salix glabra, Salix pentandra, or Salix serpyllifolia.

3. The genetically engineered methanotrophic bacterium of claim 1, wherein the exogenous nucleic acid molecule encoding the isoprene synthase (a) is codon optimized for expression in the methanotrophic bacterium, (b) does not comprise an N-terminal plastid-targeting sequence, or (c) both.

4. (canceled)

5. The genetically engineered methanotrophic bacterium of claim 2, wherein the nucleic acid encodes an amino acid sequence set forth in any one of SEQ ID NOs:1-6, 14-19.

6. (canceled)

7. The genetically engineered methanotrophic bacterium of claim 1, wherein the exogenous nucleic acid molecule encoding isoprene synthase is operatively linked to an expression control sequence selected from a methanol dehydrogenase promoter, hexulose 6-phosphate synthase promoter, ribosomal protein S16 promoter, serine hydroxymethyl transferase promoter, serine-glyoxylate aminotransferase promoter, phosphoenolpyruvate carboxylase promoter, T5 promoter, or Trc promoter.

8. (canceled)

9. The genetically engineered methanotrophic bacterium of claim 1, wherein the methanotrophic bacterium further (a) overexpresses an endogenous DXP pathway enzyme as compared to expression of the endogenous DXP pathway enzyme by a parent methanotrophic bacterium, (b) comprises and expresses an exogenous nucleic acid molecule encoding a DXP pathway enzyme, or (c) a combination thereof.

10. The genetically engineered methanotropic bacterium of claim 1, wherein the methanotropic bacterium further (a) overexpresses an endogenous mevalonate pathway enzyme as compared to expression of the endogenous mevalonate pathway enzyme by a parent methanotrophic bacterium, (b) comprises and expresses an exogenous nucleic acid molecule encoding a mevalonate pathway enzyme, or (c) a combination thereof.

11. The genetically engineered methanotrophic bacterium of claim 9, wherein the DXP pathway enzyme is DXS, DXR, IDI, IspD, IspE, IspF, IspG, IspH, or a combination thereof.

12. The genetically engineered methanotropic bacterium of claim 10, wherein the mevalonate pathway enzyme is acetoacetyl-CoA thiolase, 3-hydroxy-3-methylglutaryl-CoA synthase, 3-hydroxy-3-methylglutaryl-CoA reductase, mevalonate kinase, phophomevalonate kinase, mevalonate pyrophosphate decarboxylase, isopentenyl diphosphate isomerase, or a combination thereof.

13. The genetically engineered methanotrophic bacterium of claim 1, wherein the methanotrophic bacterium further comprises an exogenous nucleic acid molecule encoding an alternative DXP pathway enzyme.

14. The genetically engineered methanotrophic bacterium of claim 13, wherein the alternative DXP pathway enzyme is capable of rescuing a DXS-defective phenotype in the methanotrophic bacterium, wherein the encoded alternative DXP pathway enzyme is a mutant catalytic E subunit of pyruvate dehydrogenase (PDH), a mutant 3,4 dihydroxy-2-butanone 4-phosphate synthase (DHBPS), or both.

15. (canceled)

16. The genetically engineered methanotrophic bacterium according to claim 1, wherein the methanotrophic bacterium is selected from a Methylomonas, Methylobacter, Methylococcus, Methylosinus, Methylocystis, Methylomicrobium, Methanomonas, Methylocella, or Methylocapsa.

17. The genetically engineered methanotrophic bacterium of claim 1, wherein the methanotrophic bacterium is Methylococcus capsulatus Bath strain, Methylomonas methanica 16a (ATCC PTA 2402), Methylosinus trichosporium OB3b (NRRL B-11, 196), Methylosinus sporium (NRRL B-11, 197), Methylocystis parvus (NRRL B-11, 198), Methylomonas methanica (NRRL B-11, 199), Methylomonas albus (NRRL B-11, 200), Methylobacter capsulatus (NRRL B-11, 201), Methylobacterium organophilum (ATCC 27, 886), Methylomonas sp AJ-3670 (FERM P-2400), Methylocella silvestris, Methylocella palustris (ATCC 700799), Methylocella tundrae, Methylocystis daltona strain SB2, Methylocystis bryophila, Methylocapsa aurea KYG, Methylacidiphilum infernorum, Methylacidiphilum fumariolicum, Methyloacida kamchatkensis, Methylibium petroleiphilum, or Methylomicrobium alcaliphilum.

18. (canceled)

19. A method of producing isoprene, comprising culturing a genetically engineered methanotrophic bacterium comprising an exogenous nucleic acid molecule encoding isoprene synthase in the presence of a carbon feedstock under conditions sufficient to produce isoprene.

20. The method of claim 19, wherein the nucleic acid molecule encoding the isoprene synthase is an isoprene synthase of Populus alba, Populus trichocarpa, Populus tremuloides, Populus nigra, Populus alba×Populus tremula, Populus×canescens, Pueraria montana, Pueraria lobata, Quercus robur, Faboideae, Salix discolor, Salix glabra, Salix pentandra, or Salix serpyllifolia.

21. The method of claim 19, wherein the exogenous nucleic acid molecule encoding the isoprene synthase (a) is codon optimized for expression in the methanotrophic bacterium, (b) does not comprise an N-terminal plastid-targeting sequence, or (c) both.

22. (canceled)

23. The method of claim 19, wherein the exogenous nucleic acid molecule encodes an amino acid sequence set forth in any one of SEQ ID NOs:1-6, 14-19.

24. (canceled)

25. The method of claim 19, wherein the exogenous nucleic acid molecule encoding isoprene synthase is operatively linked to an expression control sequence selected from a methanol dehydrogenase promoter, hexulose 6-phosphate synthase promoter, ribosomal protein S16 promoter, serine hydroxymethyl transferase promoter, serine-glyoxylate aminotransferase promoter, phosphoenolpyruvate carboxylase promoter, T5 promoter, or Trc promoter.

26. (canceled)

27. The method of claim 19, wherein the methanotrophic bacterium further (a) overexpresses an endogenous DXP pathway enzyme as compared to expression of the endogenous DXP pathway enzyme by a parent methanotrophic bacterium, (b) comprises and expresses an exogenous nucleic acid molecule encoding a DXP pathway enzyme, or a combination thereof.

28. The method of claim 27, wherein the DXP pathway enzyme is DXS, DXR, IDI, IspD, IspE, IspF, IspG, IspH, or a combination thereof.

29-30. (canceled)

31. The method of claim 19, wherein the methanotrophic bacterium is selected from a Methylomonas, Methylobacter, Methylococcus, Methylosinus, Methylocystis, Methylomicrobium, Methanomonas, Methylocella, or Methylocapsa.

32. The method of claim 1, wherein the methanotrophic bacterium is Methylococcus capsulatus Bath strain, Methylomonas methanica 16a (ATCC PTA 2402), Methylosinus trichosporium OB3b (NRRL B-11, 196), Methylosinus sporium (NRRL B-11, 197), Methylocystis parvus (NRRL B-11, 198), Methylomonas methanica (NRRL B-11, 199), Methylomonas albus (NRRL B-11, 200), Methylobacter capsulatus (NRRL B-11, 201), Methylobacterium organophilum (ATCC 27, 886), Methylomonas sp AJ-3670 (FERM P-2400), Methylocella silvestris, Methylocella palustris (ATCC 700799), Methylocella tundrae, Methylocystis daltona strain SB2, Methylocystis bryophila, Methylocapsa aurea KYG, Methylacidiphilum infernorum, Methylacidiphilum fumariolicum, Methyloacida kamchatkensis, Methylibium petroleiphilum, or Methylomicrobium alcaliphilum.

33. The method of claim 19, wherein the carbon feedstock converted into isoprene is methane, methanol, natural gas, or unconventional natural gas.

34. (canceled)

35. The method of claim 19, wherein the methanotrophic bacterium is cultured by fermentation and the isoprene produced from the fermentation is recovered as an off-gas.

36. The method of claim 35, wherein the recovered isoprene is further modified into a dimer (10-carbon) hydrocarbon, a trimer (15-carbon) hydrocarbon, or a combination thereof.

37. The method of claim 36, wherein the dimer hydrocarbon, trimer hydrocarbon, or combination thereof is hydrogenated into long-chain branched alkanes.

38. The method of claim 35, wherein the recovered isoprene is further modified into an isoprenoid product.

39.-53. (canceled)

54. An isoprene composition, wherein the isoprene has a δ¹³C distribution less than about −30‰.

55. (canceled)

56. (canceled)

57. The isoprene composition of claim 54, wherein the isoprene has a δ¹³C distribution ranging from about −30‰ to about −50‰.

58. (canceled)

59. (canceled)
